(19) United States
(12) Patent Application Publication
(10) Pub. No.: US 2026/0152508 A1
(43) Pub. Date: Jun. 4, 2026

(54) Heteroarylsulfonyl Compounds As USP18 Inhibitors

(71) Applicant: Incyte Corporation, Wilmington, DE (US)

(72) Inventors: Joseph Barbosa, Lambertville, NJ (US); Chad Bomgardner, Wilmington, DE (US); James A. Brackley, III, Wilmington, DE (US); Stephen Douglass, Marlton, NJ (US); Hao Feng, Glen Mills, PA (US); Joseph Glenn, Mount Royal, NJ (US); Sunkyu Kim, Hockessin, DE (US); Michael S. McQueney, Philadelphia, PA (US); Guofeng Zhang, Kennett Square, PA (US)

(21) Appl. No.: 19/406,284

(22) Filed: Dec. 2, 2025

Related U.S. Application Data
(60) Provisional application No. 63/727,750, filed on Dec. 4, 2024.

(51) Int. Cl. C07D 487/10; A61K 31/4025; A61K 31/4035; A61K 31/404; A61K 31/4353; A61K 31/454; A61K 31/4709; A61K 31/4725; A61K 31/496; A61K 31/4995; A61K 31/551; C07D 401/12; C07D 401/14; C07D 403/12; C07D 403/14; C07D 405/14; C07D 409/14; C07D 471/04; C07D 487/08

(52) U.S. Cl. CPC C07D 487/10; A61K 31/4025; A61K 31/4035; A61K 31/404; A61K 31/4353; A61K 31/454; A61K 31/4709; A61K 31/4725; A61K 31/496; A61K 31/4995; A61K 31/551; C07D 401/12; C07D 401/14; C07D 403/12; C07D 403/14; C07D 405/14; C07D 409/14; C07D 471/04; C07D 487/08

(57) ABSTRACT

The present invention provides heteroarylsulfonyl compounds that modulate the activity of USP18 and are useful in the treatment of diseases related to USP18, including cancer.

### SEQUENCE LISTING

This application contains a Sequence Listing that has been submitted electronically as an XML file named “20443-0877001_SL_ST26.XML.” The XML file, created on Nov. 26, 2025, is 2,651 bytes in size. The material in the XML file is hereby incorporated by reference in its entirety.

### TECHNICAL FIELD

The present invention provides heteroarylsulfonyl compounds that modulate the activity of USP18 and are useful in the treatment of diseases related to USP18, including cancer.

### BACKGROUND

USP18 is an enzyme that primarily negatively regulates the cellular responses to type I interferons (IFN-Is). IFN-Is, such as IFN-α, and IFN-β, are important cytokines associated with innate immune response which have a major influence on a cell's defense to viral infections. IFN-Is also have profound roles in fighting against tumors. Certain tumors have evolved to survive in the presence of IFN-Is and evade cell death. As an important negative regulator of IFN-Is, USP18 itself is upregulated and expressed in response to IFN-α, and IFN-β, thus making this protein an important factor that virally infected cells and specific cancers rely on to survive.

Mechanistically, USP18 has two major functions to dampen the effects of IFN-I response on cells. First, the USP18 enzyme has a specific activity to cleave the conjugation of ISG15 from other interferon stimulated genes (ISGs), a process called de-ISGylation. The production of ISGs are important products from IFN-I stimulation that dictate the cells response to factors including viral production and oncogenic transformation. Thus, the prevention of ISG accumulation within a cell, driven by USP18, is a key mechanism for cells to adequately respond to viral and oncogenic stimuli. This catalytic function is directed primarily around two cysteines positioned at amino acids 64 and 65. The second function relies on structural inhibition by binding to the interferon alpha receptor subunit 2 (IFNAR2) and preventing further IFN-I interactions with the receptor. IFN-I binding to IFNAR2 results in the phosphorylation of JAK1/STAT1 proteins at the inner cellular membrane causing downstream signaling to produce ISGs. Once induced by IFN-I, USP18 can transport to the inner cellular membrane with STAT2 to displace JAK1 from the IFNAR2 complex and thus prevent the phosphorylation of STAT1 and abrogate downstream signaling. The interaction with STAT2 is reported to be responsible at isoleucine 61 on USP18, and thus, independent from the ISG15-specific region previously described.

Compounds in this application may have activity towards USP18. These USP18 inhibitors alone or in combination with other therapeutic agent(s) can be used in treatment of cancer.

### SUMMARY

The present invention relates to, inter alia, compounds of Formula I:

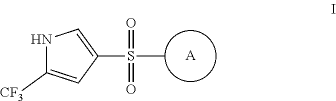

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of inhibiting an activity of USP18, comprising contacting the enzyme with a compound of Formula I, or a pharmaceutically acceptable salt thereof.

The present invention further provides methods of treating a disease or a disorder associated with expression or activity of USP18 in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DESCRIPTION OF DRAWINGS

FIG. 1 shows the compound dose response effect of Example 65 in A549 cells on ISGylated proteins by Western Blot analysis.

FIG. 2 shows the ability of USP18 inhibitors, Example 63 and Example 65, to prevent USP18 inhibition of IFNAR signaling by Western Blot analysis of downstream phosphorylated Signal Transducer and Activator of Transcription 1 (pSTAT1).

FIGS. 3A and 3B show USP18 inhibition of IFNAR signaling by HTRF detection of downstream phosphorylated Signal Transducer and Activator of Transcription 1 (pSTAT1) using the compounds of Example 63 (FIG. 3A) and Example 65 (FIG. 3B).

### DETAILED DESCRIPTION

The present application provides a compound of Formula I:

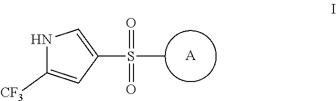

or a pharmaceutically acceptable salt thereof, wherein:
Ring A is independently selected from:

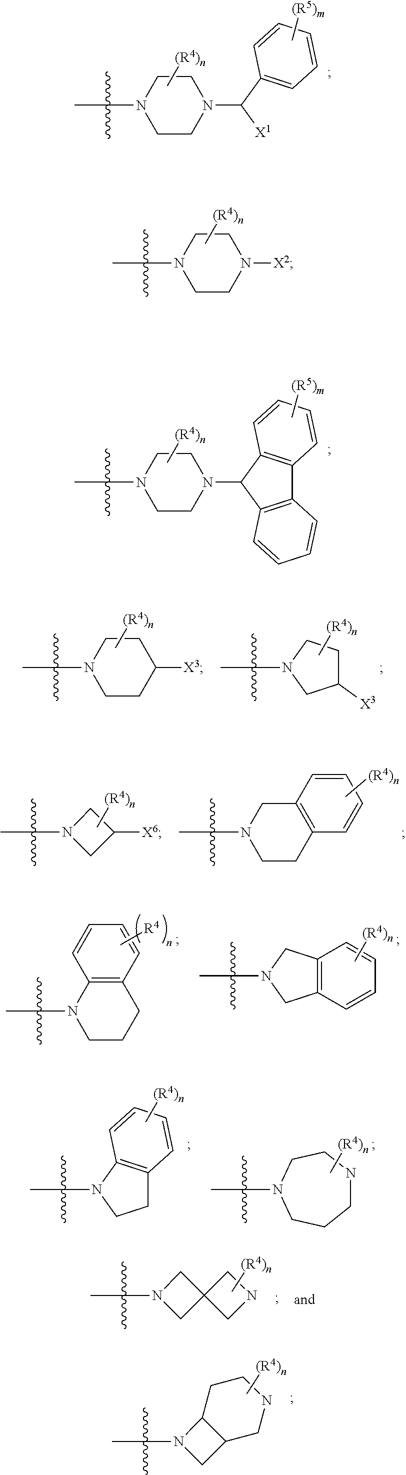

X¹ is selected from C₁₋₆ alkyl, C₆₋₁₀ aryl, C₃₋₁₀ cycloalkyl, 5-10 membered heteroaryl, 4-10 membered heterocycloalkyl, C(O)NHR^{a1}, and C(O)OR^{a1}, wherein the C₁₋₆ alkyl, C₆₋₁₀ aryl, C₃₋₁₀ cycloalkyl, 5-10 membered heteroaryl, and 4-10 membered heterocycloalkyl of X¹ are each optionally substituted with 1, 2, 3, 4, 5, 6, 7, or 8 independently selected R¹ substituents;
each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)R^{b1}, NR^{c1}S(O)NR^{c1}R^{d1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{c1}C)R^{b1}, and OS(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹ are each optionally substituted with 1, 2, 3, 4, 5, 6, 7, or 8 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a1}, R^{c1} and R^{d1} are each optionally substituted with 1, 2, 3, 4, 5, 6, 7, or 8 independently selected R^{1A} substituents;
or, any R^{c1} and R^{d1} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1A} substituents;
each R^{b1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1} are each optionally substituted with 1, 2, 3, 4, 5, 6, 7, or 8 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{c11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, 4, 5, 6, 7, or 8 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a11}, R^{c11} and R^{d11} are each optionally substituted with 1, 2, 3, 4, 5, 6, 7, or 8 independently selected R^{1B} substituents;
or, any R^{c11} and R^{d11} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1B} substituents;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b11} are each optionally substituted with 1, 2, 3, 4, 5, 6, 7, or 8 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)R^{b12}, NR^{c12}S(O)NR^{c12}R^{d12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, and OS(O)₂R^{b12}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{1B} are each optionally substituted with 1, 2, 3, or 4 independently selected R^{M} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl of R^{a12}, R^{c12} and R^{d12} are each optionally substituted with 1, 2, 3, or 4 independently selected R^{M} substituents;
or, any R^{c12} and R^{d12} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b12} are each optionally substituted with 1, 2, 3, or 4 independently selected R^{M} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl;
X² is selected from C₆₋₁₀ aryl and 5-10 membered heteroaryl, wherein the C₆₋₁₀ aryl and 5-10 membered heteroaryl of X² are each optionally substituted with 1, 2, 3, 4, 5, 6, 7, or 8 independently selected R² substituents;
each R² is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, and OS(O)₂R^{b2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl and 4-10 membered heterocycloalkyl group are each optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{2A} are each optionally substituted with 1, 2, 3, or 4 independently selected R^{M} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a21}, R^{c21} and R^{d21} are each optionally substituted with 1, 2, 3, or 4 independently selected R^{M} substituents;
or, any R^{c21} and R^{d21} attached to the same N atom, together with the N atom to which they are attached, form a 5-6 membered heteroaryl or a 4-7 membered heterocycloalkyl group, wherein the 5-6 membered heteroaryl and 4-7 membered heterocycloalkyl group are each optionally substituted with 1, 2, 3, or 4 independently selected R^{M} substituents;
each R^{b21} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b21} are each optionally substituted with 1, 2, 3, or 4 independently selected R^{M} substituents;
each R^{c21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
X³ is selected from C₆₋₁₀ aryl-C₁₋₆ alkyl- and C₆₋₁₀ aryl, wherein the C₆₋₁₀ aryl-C₁₋₆ alkyl- and C₆₋₁₀ aryl of X³ are each optionally substituted with 1, 2, 3, 4, 5, 6, 7, or 8 independently selected R³ substituents;
each R³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)NR^{c3}R^{d3}, NR^{c3}S(O)R, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, and OS(O)₂R^{b3}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, 4, 5, 6, 7, or 8 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a3}, R^{c3} and R^{d3} are each optionally substituted with 1, 2, 3, 4, 5, 6, 7, or 8 independently selected R^{3A} substituents;
or, any R^{c3} and R^{d3} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3A} substituents;
each R^{b3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b3} are each optionally substituted with 1, 2, 3, 4, 5, 6, 7, or 8 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{3A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, CN, NO₂, OR^{a31}, SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}C(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31}, NR^{c31}S(O)R^{b31}, NR^{c31}S(O)NR^{c31}R^{d31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, and OS(O)₂R^{b31}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{3A} are each optionally substituted with 1, 2, 3, or 4 independently selected R^{M} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a31}, R^{c31} and R^{d31} are each optionally substituted with 1, 2, 3, or 4 independently selected R^{M} substituents;
or, any R^{c31} and R^{d31} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b31} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b31} are each optionally substituted with 1, 2, 3, or 4 independently selected R^{M} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
each R⁴ is independently selected from H, oxo, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, and OS(O)₂R^{b4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4A} are each optionally substituted with 1, 2, 3, 4, 5, 6, 7, or 8 independently selected R^{4A} substituents;
each R^{a4}, R⁴, and R^{d4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a4}, R^{c4} and R^{d4} are each optionally substituted with 1, 2, 3, 4, 5, 6, 7, or 8 independently selected R^{4A} substituents;
or, any R^{c4} and R^{d4} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4A} substituents;
each R^{b4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b4} are each optionally substituted with 1, 2, 3, 4, 5, 6, 7, or 8 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{4A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c4}C(═NR^{e41})NR^{c41}R^{d41}, R^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, and OS(O)₂R^{b41}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{4A} are each optionally substituted with 1, 2, 3, or 4 independently selected R^{M} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a41}, R^{c41} and R^{d41} are each optionally substituted with 1, 2, 3, or 4 independently selected R^{M} substituents;
or, any R^{c41} and R^{d41} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b41} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{b41} are each optionally substituted with 1, 2, 3, or 4 independently selected R^{M} substituents;
each R^{c41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-,
or, alternatively, two R⁴ groups together form a 7-9 membered bridged bicycle with the ring to which the two R⁴ groups are attached;
each R⁵ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R⁵, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{C5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, and OS(O)₂R^{b5}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, 4, 5, 6, 7, or 8 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a5}, R^{c5} and R^{d5} are each optionally substituted with 1, 2, 3, 4, 5, 6, 7, or 8 independently selected R^{5A} substituents;
or, any R^{c5} and R^{d5} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{5A} substituents;
each R^{b5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b5} are each optionally substituted with 1, 2, 3, 4, 5, 6, 7, or 8 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{5A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)R^{b51}, NR^{c51}S(O)NR^{c51}R^{d51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, and OS(O)₂R^{b51}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{5A} are each optionally substituted with 1, 2, 3, or 4 independently selected R^{M} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a51}, R^{c51} and R^{d51} are each optionally substituted with 1, 2, 3, or 4 independently selected R^{M} substituents;
or, any R^{c51} and R^{d51} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b51} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{b51} are each optionally substituted with 1, 2, 3, or 4 independently selected R^{M} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-,
X⁶ is selected from C₆₋₁₀ aryl-C₁₋₆ alkyl-, C₆₋₁₀ aryl and 5-10 membered heteroaryl, wherein the C₆₋₁₀ aryl-C₁₋₆ alkyl-, C₆₋₁₀ aryl and 5-10 membered heteroaryl of X⁶ are each optionally substituted with 1, 2, 3, 4, 5, 6, 7, or 8 independently selected R⁶ substituents;
each R⁶ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}(O)OR^{a6}, NR^{c6}C(O)NR^{c6}R^{d6}, C(═NR^{e6})R^{b6}, C(═NOH)R^{b6}, C(═NCN)R^{b6}, C(═NR^{e6})NR^{c6}R^{d6}, NR^{c6}C(═NR^{e6})NR^{c6}R^{d6}, NR^{c6}C(═NOH)NR^{c6}R^{d6}, NR^{c6}C(═NCN)NR^{c6}R⁶, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, S(O)(═NR^{e6})R^{b6}, OS(O)(═NR^{e6})R^{b6}, and OS(O)₂R^{b6}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁶ are each optionally substituted with 1, 2, 3, 4, 5, 6, 7, or 8 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a6}, R^{c6} and R^{d6} are each optionally substituted with 1, 2, 3, 4, 5, 6, 7, or 8 independently selected R^{6A} substituents;
or, any R^{c6} and R^{d6}, attached to the same N atom, together with the N atom to which they are attached, form a 5-10 membered heteroaryl group or a 4-10 membered heterocycloalkyl group, wherein the 5-10 membered heteroaryl group or 4-10 membered heterocycloalkyl group is optionally substituted with 1, 2, 3, 4, 5, 6, 7, or 8 independently selected R^{6A} substituents;
each R^{b6} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b6} are each optionally substituted with 1, 2, 3, 4, 5, 6, 7, or 8 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{6A} is selected from H, halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R⁶¹, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NOH)R^{b61}, C(═NCN)R^{b61}, C(═NR^{e61})NR^{c61}R^{d61}, NR^{c61}C(═NR^{e61})NR^{c61}R^{d61}, NR^{c61}C(═NOH)NR^{c61}R^{d61}, NR^{c61}C(═NCN)NR^{c61}R^{d61}, NR^{c61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, S(O)(═NR^{e61})R^{b61}, OS(O)(═NR^{e61})R^{b61}, and OS(O)₂R^{b61}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{6A} are each optionally substituted with 1, 2, 3, or 4 independently selected R^{M} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{a61}, R^{c61} and R^{d61} are each optionally substituted with 1, 2, 3, or 4 independently selected R^{M} substituents;
or, any R^{c61} and R^{d61}, attached to the same N atom, together with the N atom to which they are attached, form a 5-6 membered heteroaryl group or a 4-7 membered heterocycloalkyl group, wherein the 5-6 membered heteroaryl group or 4-7 membered heterocycloalkyl group is optionally substituted with 1, 2, 3, or 4 independently selected R^{M} substituents;
each R^{b6} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{b6} are each optionally substituted with 1, 2, 3, or 4 independently selected R^{M} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-;
n is 0, 1, or 2;
m is 0, 1, or 2; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-; and
provided that X² is not 2-chlorophenyl.

In some embodiments, Ring A is

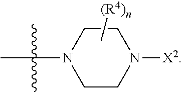

In some embodiments, X² is selected from C₆₋₁₀ aryl and 5-10 membered heteroaryl, wherein the C₆₋₁₀ aryl and 5-10 membered heteroaryl of X² are each optionally substituted with 1, 2, 3, or 4 independently selected R² substituents.

In some embodiments, X² is selected from C₆₋₁₀ aryl and 5-10 membered heteroaryl, wherein the C₆₋₁₀ aryl and 5-10 membered heteroaryl of X² are each optionally substituted with 1 or 2 independently selected R² substituents.

In some embodiments, X² is selected from phenyl, naphthalenyl, and pyridinyl, wherein the phenyl, naphthalenyl, and pyridinyl of X² are each optionally substituted with 1, 2, 3, or 4 independently selected R² substituents.

In some embodiments, X² is selected from phenyl, naphthalenyl, and pyridinyl, wherein the phenyl, naphthalenyl, and pyridinyl of X² are each optionally substituted with 1 or 2 independently selected R² substituents.

In some embodiments, each R² is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, and NR^{c2}R^{d2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents.

In some embodiments, each R² is independently selected from halo, C₁₋₆ alkyl, C₁₋₆ haloalkyl, C₆₋₁₀ aryl, 5-10 membered heteroaryl, CN, NO₂, OR^{a2}, C(O)NR^{c2}R^{d2}, C(O)OR^{a2}, and NR^{c2}R^{d2}, wherein the C₁₋₆ alkyl, C₆₋₁₀ aryl, and 5-10 membered heteroaryl of R² are each optionally substituted with 1, 2, 3, or 4 independently selected R^{2A} substituents; and
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{c2} and R^{d2} are each optionally substituted with 1 or 2 independently selected R^{2A} substituents.

In some embodiments, each R² is independently selected from halo, C₁₋₆ alkyl, C₁₋₆ haloalkyl, C₆₋₁₀ aryl, 5-10 membered heteroaryl, CN, NO₂, OR^{a2}, C(O)NR^{c2}R^{d2}, C(O)OR^{a2}, and NR^{c2}R^{d2}, wherein the C₁₋₆ alkyl, C₆₋₁₀ aryl, and 5-10 membered heteroaryl of R² are each optionally substituted with 1, 2, 3, or 4 independently selected R^{2A} substituents; and
each R^{a2}, R^{c2}, and R^{d2} is independently selected from H, C₁₋₆ alkyl, and phenyl, wherein the C₁₋₆ alkyl and phenyl of R^{a2}, R^{c2} and R^{d2} are each optionally substituted with 1 or 2 independently selected R^{2A} substituents.

In some embodiments, each R² is independently selected from halo, C₁₋₆ alkyl, C₁₋₆ haloalkyl, C₆₋₁₀ aryl, 5-10 membered heteroaryl, CN, NO₂, OR^{a2}, C(O)NR^{c2}R^{d2}, C(O)OR^{a2}, and NR^{c2}R^{d2}, wherein the C₁₋₆ alkyl, C₆₋₁₀ aryl, and 5-10 membered heteroaryl of R² are each optionally substituted with 1, 2, 3, or 4 independently selected R^{2A} substituents.

In some embodiments, each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{c2} and R^{d2} are each optionally substituted with 1 or 2 independently selected R^{2A} substituents.

In some embodiments, each R^{a2}, R^{c2}, and R^{d2} is independently selected from H, C₁₋₆ alkyl, and C₆₋₁₀ aryl, wherein the C₁₋₆ alkyl and C₆₋₁₀ aryl of R^{a2}, R^{c2} and R^{d2} are each optionally substituted with 1 or 2 independently selected R^{2A} substituents.

In some embodiments, each R^{a2}, R^{c2}, and R^{d2} is independently selected from H, methyl, and phenyl, wherein the phenyl is optionally substituted with halo.

In some embodiments, each R² is independently selected from fluoro, chloro, bromo, methyl, ethyl, trifluoromethyl, phenyl, pyrazolyl, pyridinyl, OCH₃, NH₂, C(O)OCH₃, CN, NO₂, and (fluorophenyl)carbamyl, wherein the methyl, ethyl, trifluoromethyl, phenyl, pyrazolyl, and pyridinyl are each optionally substituted with 1 or 2 independently selected R^{2A} substituents.

In some embodiments, each R^{2A} is independently selected from halo, C₁₋₆ alkyl, C₁₋₆ haloalkyl, C₂₋₆ alkenyl, and C₂₋₆ alkynyl.

In some embodiments, each R^{2A} is independently selected from halo and C₁₋₆ alkyl.

In some embodiments, each R² is independently selected from methyl, ethyl, fluoro, chloro, bromo, trifluoromethyl, phenyl, OCH₃, NH₂, C(O)OCH₃, CN, NO₂, (fluorophenyl)carbamyl, methylpyrazolyl, and pyridinyl.

In some embodiments, X² is (chloro)(methyl)phenyl, nitrophenyl, dichlorophenyl, phenyl, ethylphenyl, (fluoro)(methyl)phenyl, (trifluoromethyl)phenyl, (methyl)(phenyl)phenyl, (phenyl)phenyl, cyanophenyl, methoxyphenyl, aminophenyl, naphthalenyl, (methoxycarbonylmethyl)phenyl, dichloropyridinyl, (chloro)(trifluoromethyl)pyridinyl, (bromo)(methyl)phenyl, ((fluorophenyl)carbamyl)(chloro)phenyl, (methylpyrazolyl)phenyl, and (methyl)(pyridinyl)phenyl.

In some embodiments, X² is 5-chloro-2-methylphenyl, 3-nitrophenyl, 2-chloro-6-methylphenyl, 2,4-dichlorophenyl, 2,6-dichlorophenyl, 4-chloro-2-methylphenyl, phenyl, 2-ethylphenyl, 4-fluoro-2-methylphenyl, 5-trifluoromethylphenyl, 3-chloro-2-methylphenyl, (2-methyl)(4-phenyl)phenyl, 2-phenyl-phenyl, 2-cyanophenyl, 2-methoxyphenyl, 2-aminophenyl, naphthalen-1-yl, 3-methoxycarbonyl-6-methyl-phenyl, 3,5-dichloropyridin-2-yl, 3,5-dichloropyridin-4-yl, 3-chloro-5-(trifluoromethyl)pyridin-2-yl, 5-bromo-2-methylphenyl, 4-(4-fluorophenyl)carbamyl-2-chlorophenyl, (1-methyl-1H-pyrazol-4-yl)phenyl, 2-methyl-5-(pyridin-4-yl)phenyl, and 2-methyl-5-(pyridin-3-yl)phenyl.

In some embodiments, each R⁴ is independently selected from H, halo, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R⁴ are each optionally substituted with 1, 2, or 3 independently selected R^{4A} substituents,
or, alternatively, two R⁴ groups together form a 7-9 membered bridged bicycle with the ring to which the two R⁴ groups are attached.

In some embodiments, each R⁴ is C₁₋₆ alkyl, wherein the C₁₋₆ alkyl is optionally substituted with 1, 2, or 3 independently selected R^{4A} substituents,
or, alternatively, two R⁴ groups together form a 7 membered bridged bicycle with the ring to which the two R⁴ groups are attached; and
each R^{4A} is hydroxy.

In some embodiments, each R⁴ is C₁₋₆ alkyl, wherein the C₁₋₆ alkyl is optionally substituted with 1, 2, or 3 independently selected R^{4A} substituents,
or, alternatively, two R⁴ groups together form a 7 membered bridged bicycle with the ring to which the two R⁴ groups are attached.

In some embodiments, each R^{4A} is hydroxy.

In some embodiments, each R⁴ is independently selected from methyl and hydroxymethyl.

In some embodiments, Ring A is

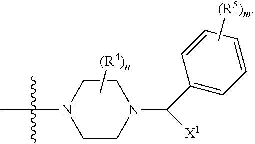

In some embodiments, each R⁵ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)NR^{c5}R^{d5}, NR^{c5}S(O)₂R^{b5}, and S(O)₂R^{b5}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R⁵ are each optionally substituted with 1, 2, 3, or 4 independently selected R^{5A} substituents; and
each R^{a5}, R^{b5}, R^{c5}, and R^{d5} is independently selected from H, C₁₋₆ alkyl, C₁₋₆ haloalkyl, C₂₋₆ alkenyl, and C₂₋₆ alkynyl.

In some embodiments, each R⁵ is independently selected from halo, C₁₋₆ alkyl, C₁₋₆ haloalkyl, C₆₋₁₀ aryl, 5-10 membered heteroaryl, CN, OR^{a5}, C(O)R^{b5}, C(O)NR^{c5}R^{d5}, C(O)OR^{a5}, NR^{c5}R^{d5}, NR^{c5}S(O)₂R^{b5}, and S(O)₂R^{b5}, wherein the C₁₋₆ alkyl, C₆₋₁₀ aryl, and 5-10 membered heteroaryl of R⁵ are each optionally substituted with 1, 2, 3, or 4 independently selected R^{5A} substituents; and
each R^{a5}, R^{b5}, R^{c5}, and R^{d5} is independently selected from H and C₁₋₆ alkyl.

In some embodiments, each R⁵ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)NR^{c5}R^{d5}, NR^{c5}S(O)₂R^{b5}, and S(O)₂R^{b5}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R⁵ are each optionally substituted with 1, 2, 3, or 4 independently selected R^{5A} substituents.

In some embodiments, each R⁵ is independently selected from halo, C₁₋₆ alkyl, C₁₋₆ haloalkyl, C₆₋₁₀ aryl, 5-10 membered heteroaryl, CN, OR^{a5}, C(O)R^{b5}, C(O)NR^{c5}R^{d5}, C(O)OR^{a5}, NR^{c5}R^{d5}, NR^{c5}S(O)₂R^{b5}, and S(O)₂R^{b5}, wherein the C₁₋₆ alkyl, C₆₋₁₀ aryl, and 5-10 membered heteroaryl of R⁵ are each optionally substituted with 1, 2, 3, or 4 independently selected R^{5A} substituents.

In some embodiments, each R^{a5}, R^{b5}, R^{c5}, and R^{d5} is independently selected from H, C₁₋₆ alkyl, C₁₋₆ haloalkyl, C₂₋₆ alkenyl, and C₂₋₆ alkynyl.

In some embodiments, each R^{a5}, R^{b5}, R^{c5}, and R^{d5} is independently selected from H and C₁₋₆ alkyl.

In some embodiments, each R^{a5}, R^{b5}, R^{c5}, and R^{d5} is independently selected from H and methyl.

In some embodiments, each R^{a5}, R^{c5}, and R^{d5} is independently selected from H, C₁₋₆ alkyl, C₁₋₆ haloalkyl, C₂₋₆ alkenyl, and C₂₋₆ alkynyl.

In some embodiments, each R^{a5}, R^{c5}, and R^{d5} is independently selected from H and C₁₋₆ alkyl.

In some embodiments, each R^{a5}, R^{c5}, and R^{d5} is independently selected from H and methyl.

In some embodiments, each R^{b5} is independently selected from H, C₁₋₆ alkyl, C₁₋₆ haloalkyl, C₂₋₆ alkenyl, and C₂₋₆ alkynyl.

In some embodiments, each R^{b5} is independently selected from H and C₁₋₆ alkyl.

In some embodiments, each R^{b5} is independently selected from H and methyl.

In some embodiments, each R^{5A} is independently selected from CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, CNR^{c51}S(O)R^{b51}, NR^{c51}S(O)NR^{c51}R^{d51}, and NR^{c51}S(O)₂R^{b51};
each R^{a51}, R^{c51}, and R^{d51} is independently selected from H, C₁₋₆ alkyl, C₁₋₆ haloalkyl, C₂₋₆ alkenyl, and C₂₋₆ alkynyl;
each R^{b51} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R^{b51} are each optionally substituted with 1 or 2 independently selected R^{M} substituents; and
each R^{M} is C₁₋₆ alkyl, C₁₋₆ haloalkyl, C₂₋₆ alkenyl, and C₂₋₆ alkynyl.

In some embodiments, each R^{5A} is independently selected from OR^{a51}, NR^{c51}R^{d51}, NR^{c51}C(O)R^{c51}, NR^{c51}C(O)NR^{c51}R^{d51}, and NR^{c51}S(O)₂R^{b51};
each R^{a51}, R^{c51}, and R^{d51} is independently selected from H, and C₁₋₆ alkyl; and
each R^{b51} is independently selected from C₁₋₆ alkyl and 5-6 membered heteroaryl, wherein the 5-6 membered heteroaryl is optionally substituted with 1 or 2 independently selected R^{M} substituents; and
each R^{M} is C₁₋₆ haloalkyl.

In some embodiments, each R^{5A} is independently selected from CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51} NR^{c51}NR^{c51}R^{d51}, NR^{c51}C(O)R^{b51}, NR^{c51}C(O)OR^{a51}, NR^{c51}C(O)NR^{c51}R^{d51}, CNR^{c51}S(O)R^{b51}, R^{c51}S(O)NR^{c51}R^{d51}, and NR^{c51}S(O)₂R^{b51}.

In some embodiments, each R^{5A} is independently selected from OR^{a51}, NR^{c51}R^{d51}, NR^{c51}C(O)R^{b51}, NR^{c51}C(O)NR^{c51}R^{d51}, and NR^{c51}S(O)₂R^{b51}.

In some embodiments, each R^{a51}, R^{c51}, and R^{c51} is independently selected from H, and C₁₋₆ alkyl.

In some embodiments, each R^{a51}, R^{c51}, and R^{d51} is independently selected from H, C₁₋₆ alkyl, C₁₋₆ haloalkyl, C₂₋₆ alkenyl, and C₂₋₆ alkynyl.

In some embodiments, each R^{a51}, R^{c51}, and R^{d51} is independently selected from H, and C₁₋₆ alkyl.

In some embodiments, each R^{a51}, R^{c51} and R^{d51} is independently selected from H and methyl.

In some embodiments, each R^{b51} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R^{b51} are each optionally substituted with 1 or 2 independently selected R^{M} substituents.

In some embodiments, each R^{b51} is independently selected from C₁₋₆ alkyl and 5-6 membered heteroaryl, wherein the 5-6 membered heteroaryl is optionally substituted with 1 or 2 independently selected R^{M} substituents.

In some embodiments, each R^{b51} is independently selected from methyl, pyrrolyl, and pyridinyl, wherein the pyrrole is optionally substituted with 1 or 2 independently selected R^{M} substituents.

In some embodiments, each R^{M} is C₁₋₆ alkyl, C₁₋₆ haloalkyl, C₂₋₆ alkenyl, and C₂₋₆ alkynyl.

In some embodiments, each R^{M} is C₁₋₆ haloalkyl.

In some embodiments, each R^{M} is trifluoromethyl.

In some embodiments, each R⁵ is selected from fluoro, chloro, bromo, trifluoromethyl, CN, methyl, phenyl, methoxy, methylsulfonyl, methoxycarbonyl, carboxy, formyl, hydroxymethyl, carbamyl, tetrazolyl, (trifluoromethylpyrrolyl)sulfonylaminomethyl, aminomethyl, methylcarbamylaminomethyl, pyridinylcarbonylaminomethyl, and methylaminomethyl.

In some embodiments, X¹ is selected from C₁₋₆ alkyl, C₆₋₁₀ aryl, C₃₋₁₀ cycloalkyl, 5-10 membered heteroaryl, 4-10 membered heterocycloalkyl, C(O)NHR^{a1}, and C(O)OR^{a1}, wherein the C₁₋₆ alkyl, C₆₋₁₀ aryl, C₃₋₁₀ cycloalkyl, 5-10 membered heteroaryl, and 4-10 membered heterocycloalkyl of X¹ are each optionally substituted with 1, 2, 3, or 4 independently selected R¹ substituents; and
each R¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, X¹ is selected from C₁₋₆ alkyl, phenyl, C₃₋₇ cycloalkyl, 5-6 membered heteroaryl, 4-7 membered heterocycloalkyl, C(O)NHR^{a1}, and C(O)OR^{a1}, wherein the C₁₋₆ alkyl, phenyl, C₃₋₇ cycloalkyl, 5-6 membered heteroaryl, and 4-7 membered heterocycloalkyl of X¹ are each optionally substituted with 1, 2, 3, or 4 independently selected R¹ substituents; and
each R^{a1} is independently selected from H, C₁₋₆ alkyl, C₆₋₁₀ aryl, and C₆₋₁₀ aryl-C₁₋₆ alkyl-.

In some embodiments, X¹ is selected from C₁₋₆ alkyl, C₆₋₁₀ aryl, C₃₋₁₀ cycloalkyl, 5-10 membered heteroaryl, 4-10 membered heterocycloalkyl, C(O)NHR^{a1}, and C(O)OR^{a1}, wherein the C₁₋₆ alkyl, C₆₋₁₀ aryl, C₃₋₁₀ cycloalkyl, 5-10 membered heteroaryl, and 4-10 membered heterocycloalkyl of X¹ are each optionally substituted with 1, 2, 3, or 4 independently selected R¹ substituents.

In some embodiments, X¹ is selected from C₁₋₆ alkyl, phenyl, C₃₋₇ cycloalkyl, 5-6 membered heteroaryl, 4-7 membered heterocycloalkyl, C(O)NHR^{a1}, and C(O)OR^{a1}, wherein the C₁₋₆ alkyl, phenyl, C₃₋₇ cycloalkyl, 5-6 membered heteroaryl, and 4-7 membered heterocycloalkyl of X¹ are each optionally substituted with 1, 2, 3, or 4 independently selected R¹ substituents.

In some embodiments, each R^{a1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, each R^{a1} is independently selected from H, C₁₋₆ alkyl, C₆₋₁₀ aryl, and C₆₋₁₀ aryl-C₁₋₆ alkyl-.

In some embodiments, X¹ is selected from propyl, pentyl, phenyl, cyclopropyl, cyclobutyl, cyclopentyl, cyclohexyl, pyridinyl, thiopheneyl, phenylcarbamyl, and benzylcarbamyl, wherein the phenyl is optionally substituted with 1, 2, 3, or 4 independently selected R¹ substituents.

In some embodiments, each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)NR^{c1}R^{c1}, NR^{c1}S(O)₂R^{b1}, and S(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R¹ are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-; and
each R^{b1} is independently selected from H, C₁₋₆ alkyl, C₁₋₆ haloalkyl, C₂₋₆ alkenyl, and C₂₋₆ alkynyl.

In some embodiments, each R¹ is independently selected from halo, C₁₋₆ alkyl, C₁₋₆ haloalkyl, C₆₋₁₀ aryl, 5-10 membered heteroaryl, CN, OR^{a1}, C(O)R^{b1}, C(O)NR^{c1}R^{d1}, C(O)OR^{a1}, NR^{c1}R^{d1}, NR^{c1}S(O)₂R^{b1}, and S(O)₂R^{b1}, wherein the C₁₋₆ alkyl, C₆₋₁₀ aryl, and 5-10 membered heteroaryl, of R¹ are each optionally substituted with 1, 2, 3, or 4 independently selected R^{1A} substituents;
each R^{a1}, R^{c1}, and R^{d1} is independently selected from H, C₁₋₆ alkyl, C₆₋₁₀ aryl, and C₆₋₁₀ aryl-C₁₋₆ alkyl-; and
each R^{b1} is independently selected from H and C₁₋₆ alkyl.

In some embodiments, each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)NR^{c1}R^{d1}, NR^{c1}S(O)₂R^{b1}, and S(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R¹ are each optionally substituted with 1, 2, 3, or 4 independently selected R^{1A} substituents.

In some embodiments, each R¹ is independently selected from halo, C₁₋₆ alkyl, C₁₋₆ haloalkyl, C₆₋₁₀ aryl, 5-10 membered heteroaryl, CN, OR^{a1}, C(O)R^{b1}, C(O)NR^{c1}R^{d1}, C(O)OR^{a1}, NR^{c1}R^{d1}, NR^{c1}S(O)₂R^{b1}, and S(O)₂R^{b1}, wherein the C₁₋₆ alkyl, C₆₋₁₀ aryl, and 5-10 membered heteroaryl, of R¹ are each optionally substituted with 1, 2, 3, or 4 independently selected R^{1A} substituents.

In some embodiments, each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, each R^{a1}, R^{c1}, and R^{d1} is independently selected from H, C₁₋₆ alkyl, C₆₋₁₀ aryl, and C₆₋₁₀ aryl-C₁₋₆ alkyl-.

In some embodiments, each R^{a1}, R^{c1}, and R^{d1} is independently selected from H, methyl, phenyl, and benzyl.

In some embodiments, each R^{b1} is independently selected from H, C₁₋₆ alkyl, C₁₋₆ haloalkyl, C₂₋₆ alkenyl, and C₂₋₆ alkynyl.

In some embodiments, each R^{b1} is independently selected from H and C₁₋₆ alkyl.

In some embodiments, each R^{b1} is independently selected from H and methyl.

In some embodiments, each R^{1A} is independently selected from CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}R^{d11}, NR^{c11}C(OR)^{b11}, NR^{c11}C(O)OR^{a11}, NR^{c11}C(O)NR^{c11}R^{d11}, CNR^{c11}S(O)R^{b11}, NR^{c11}S(O)NR^{c11}R^{d11}, and NR^{c11}S(O)₂R^{b11};
each R^{a11}, R^{c11}, and R^{d11} is independently selected from H, C₁₋₆ alkyl, C₁₋₆ haloalkyl, C₂₋₆ alkenyl, and C₂₋₆ alkynyl;
each R^{b11} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R^{b11} are each optionally substituted with 1 or 2 independently selected R^{1B} substituents; and
each R^{1B} is independently selected from C₁₋₆ alkyl, C₁₋₆ haloalkyl, C₂₋₆ alkenyl, and C₂₋₆ alkynyl.

In some embodiments, each R^{1A} is independently selected from OR^{a11}, NR^{c11}R^{d11}, NRCC(O)R^{b11}, NR^{c11}C(O)NR^{c11}R^{d11}, and NR^{c11}S(O)₂R^{b11};
each R^{a11}, R^{c11}, and R^{d11} is independently selected from H and C₁₋₆ alkyl;
each R^{b11} is independently selected from C₁₋₆ alkyl and 5-6 membered heteroaryl, wherein the 5-6 membered heteroaryl is optionally substituted with 1 or 2 independently selected R^{1B} substituents; and
each R^{1B} is independently selected from C₁₋₆ haloalkyl.

In some embodiments, each R^{1A} is independently selected from CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, CNR^{c11}S(O)R^{b11}, NR^{c11}S(O)NR^{c11}R^{d11}, and NR^{c11}S(O)₂R^{b11}; and

In some embodiments, each R^{1A} is independently selected from OR^{a11}, NR^{c11}R^{d11}, NR^{c11}C(O)R^{b11}, NR^{c11}C(O)NR^{c11}R^{d11}, and NR^{c11}S(O)₂R^{b11}.

In some embodiments, each R^{a11}, R^{c11}, and R^{d11} is independently selected from H, C₁₋₆ alkyl, C₁₋₆ haloalkyl, C₂₋₆ alkenyl, and C₂₋₆ alkynyl.

In some embodiments, each R^{a11}, R^{c11}, and R^{d11} is independently selected from H and C₁₋₆ alkyl.

In some embodiments, each R^{c11} and R^{d11} is independently selected from H and methyl.

In some embodiments, each R^{b11} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R^{b11} are each optionally substituted with 1 or 2 independently selected R^{1B} substituents.

In some embodiments, each R^{b11} is independently selected from C₁₋₆ alkyl and 5-6 membered heteroaryl, wherein the 5-6 membered heteroaryl is optionally substituted with 1 or 2 independently selected R^{1B} substituents.

In some embodiments, each R^{b11} is independently selected from methyl, pyrrolyl, and pyridinyl, wherein the pyrrole is optionally substituted with 1 or 2 independently selected R^{1B} substituents.

In some embodiments, each R^{1B} is independently selected from C₁₋₆ alkyl, C₁₋₆ haloalkyl, C₂₋₆ alkenyl, and C₂₋₆ alkynyl.

In some embodiments, each R^{1B} is independently selected from C₁₋₆ haloalkyl.

In some embodiments, each R^{1B} is trifluoromethyl.

In some embodiments, each R¹ is selected from fluoro, chloro, bromo, methyl, hydroxymethyl, trifluoromethyl, phenyl, tetrazolyl, methoxy, CN, methylsulfonyl, carboxy, methylcarboxy, formyl, carbamyl, aminomethyl, methylaminomethyl, ((trifluoromethyl)pyrrolesulfonyl)aminomethyl, methylcarbamylaminomethyl, and pyridinylcarbonylaminomethyl.

In some embodiments, X¹ is propyl, pentyl, phenyl, cyclopropyl, cyclobutyl, cyclopentyl, cyclohexyl, fluorophenyl, chlorophenyl, bromophenyl, methylphenyl, (trifluoromethyl)phenyl, phenyl-phenyl, (tetrazolyl)phenyl, cyanophenyl, methoxyphenyl, methoxyphenyl, (methylsulfonyl)phenyl, methylcarboxyphenyl, carboxyphenyl, formylphenyl, hydroxymethylphenyl, carbamylphenyl, ((trifluoromethyl)pyrrolesulfonylaminomethyl)phenyl, (aminomethyl)phenyl, (methylaminomethyl)phenyl, methylcarbamylaminomethylphenyl, (pyridinylcarbonylaminomethyl)phenyl, pyridinyl, thiopheneyl, phenylcarbamyl, and benzylcarbamyl.

In some embodiments, X¹ is phenyl, 4-fluorophenyl, 4-chlorophenyl, 2-chlorophenyl, pyridin-2-yl, 4-cyanophenyl, 4-bromophenyl, 2-bromophenyl, 4-(trifluoromethyl)phenyl, 2-fluorophenyl, 4-methoxyphenyl, 4-methylphenyl, 4-(phenyl)phenyl, 2-methylphenyl, 3-methoxyphenyl, 3-bromophenyl, 4-(methylsulfonyl)phenyl, thiophen-2-yl, 4-methylcarboxyphenyl, 4-carboxyphenyl, cyclopentyl, penta-3-yl, cyclobutyl, cyclohexyl, prop-2-yl, cyclopropyl, phenylcarbamyl, benzylcarbamyl, 4-formylphenyl, 4-hydroxymethylphenyl, 4-carbamylphenyl, 4-(1H-tetrazol-5-yl)phenyl, 4-(5-(trifluoromethyl)-1H-pyrrole-3-sulfonylaminomethyl)phenyl, 4-(aminomethyl)phenyl, 4-methylcarbamylaminomethylphenyl, 4-(pyridin-4-yl-carbonylaminomethyl)phenyl, and 4-(methylaminomethyl)phenyl.

In some embodiments, Ring A is selected from:

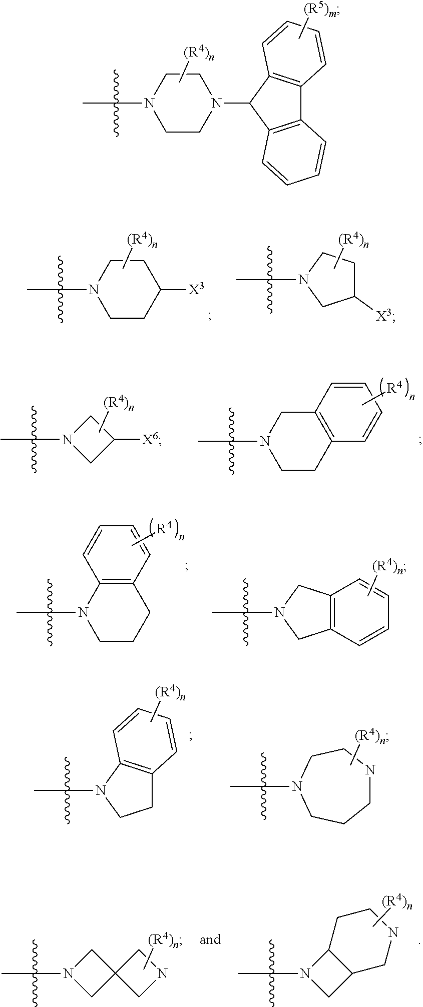

In some embodiments, each R⁴ is independently selected from H, halo, C₆₋₁₀ aryl, C₃₋₁₀ cycloalkyl, 5-10 membered heteroaryl, 4-10 membered heterocycloalkyl, and NR^{c4}C(O)OR^{a4}, wherein the C₆₋₁₀ aryl, C₃₋₁₀ cycloalkyl, 5-10 membered heteroaryl, and 4-10 membered heterocycloalkyl of R⁴ are each optionally substituted with 1 or 2 independently selected R^{4A} substituents; and
each R^{a4} and R^{c4} is independently selected from H and C₁₋₆ alkyl.

In some embodiments, each R⁴ is independently selected from H, halo, C₆₋₁₀ aryl, 4-10 membered heterocycloalkyl, and NR^{c4}C(O)OR^{a4}, wherein the C₆₋₁₀ aryl of R⁴ is each optionally substituted with 1 or 2 independently selected R^{4A} substituents; and
each R^{a4} and R^{c4} is independently selected from H and C₁₋₆ alkyl.

In some embodiments, each R⁴ is independently selected from H, fluoro, chloro, bromo, phenyl, chromanyl, and NHC(O)OR^{a4}, wherein the phenyl and chromanyl of R⁴ are each optionally substituted with 1 or 2 independently selected R^{4A} substituents; and
each R^{a4} is independently selected from H and C₁₋₆ alkyl.

In some embodiments, each R^{4A} is independently selected from halo, C₁₋₆ alkyl, C₁₋₆ haloalkyl, C₂₋₆ alkenyl, C₂₋₆ alkynyl, CN, NO₂, C(O)NR^{c41}R^{d41}, C(O)OR^{a41}, and NR^{c41}R^{d41}; and
each R^{a41}, R^{c41}, and R^{d41} is independently selected from H, C₁₋₆ alkyl, C₁₋₆ haloalkyl, C₂₋₆ alkenyl, C₂₋₆ alkynyl.

In some embodiments, each R^{4A} is independently selected from halo, C₁₋₆ alkyl, C(O)OR^{a41}, and NR^{c41}R^{d41}; and
each R^{a41}, R^{c41}, and R^{d41} is independently selected from H and C₁₋₆ alkyl.

In some embodiments, each R^{4A} is independently selected from halo, C₁₋₆ alkyl, C₁₋₆ haloalkyl, C₂₋₆ alkenyl, C₂₋₆ alkynyl, CN, NO₂, C(O)NR^{c41}R^{d41}, C(O)OR^{a41}, and NR^{c41}R^{d41}.

In some embodiments, each R^{4A} is independently selected from halo, C₁₋₆ alkyl, C(O)OR^{a41}, and NR^{c41}R^{d41}.

In some embodiments, each R^{4A} is independently selected from fluoro, chloro, bromo, methyl, dimethylamino, and methoxy.

In some embodiment, each R^{a41}, R^{c41}, and R^{d41} is independently selected from H, C₁₋₆ alkyl, C₁₋₆ haloalkyl, C₂₋₆ alkenyl, C₂₋₆ alkynyl.

In some embodiments, each R^{a41}, R^{c41}, and R^{d41} is independently selected from H and C₁₋₆ alkyl.

In some embodiments, each R⁴ is independently selected from H, fluoro, chloro, bromo, phenyl, methylphenyl, fluoromethylphenyl, N,N-dimethylaminophenyl, methoxycarbonylphenyl, chloromethylphenyl, chromanyl, and (tertbutylcarboxy)amino.

In some embodiments, X³ is selected from C₆₋₁₀ aryl and C₆₋₁₀ aryl-C₁₋₆ alkyl-, wherein the C₆₋₁₀ aryl and C₆₋₁₀ aryl-C₁₋₆ alkyl- of X³ are each optionally substituted with 1 or 2 independently selected R³ substituents.

In some embodiments, X³ is selected from phenyl and phenylmethyl, wherein the phenyl and phenylmethyl are each optionally substituted with 1 or 2 independently selected R³ substituents.

In some embodiments, each R³ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R³ are each optionally substituted with 1, 2, 3, or 4 independently selected R^{3A} substituents.

In some embodiments, each R³ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, each R³ is independently selected from halo, C₁₋₆ alkyl, and C₆₋₁₀ aryl.

In some embodiments, each R³ is independently selected from fluoro, chloro, bromo, methyl, and phenyl.

In some embodiments, X⁶ is C₆₋₁₀ aryl-C₁₋₆ alkyl-, wherein the C₆₋₁₀ aryl-C₁₋₆ alkyl- is optionally substituted with 1 or 2 independently selected R⁶ substituents.

In some embodiments, X⁶ is phenylmethyl, wherein the phenylmethyl is optionally substituted with 1 or 2 independently selected R⁶ substituents.

In some embodiments, each R⁶ is C₆₋₁₀ aryl.

In some embodiments, X⁶ is diphenylmethyl.

In some embodiments, each R⁶ is phenyl.

In some embodiments, Ring A is

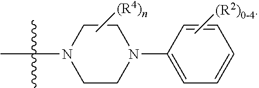

In some embodiments, Ring A is

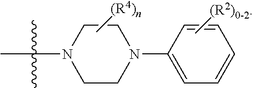

In some embodiments, Ring A is

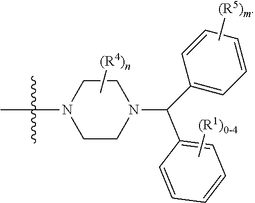

In some embodiments, Ring A is

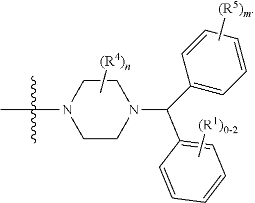

In some embodiments, Ring A is

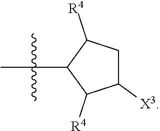

In some embodiments:
Ring A is

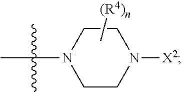

n is 0, 1, or 2;
X² is selected from C₆₋₁₀ aryl and 5-10 membered heteroaryl, wherein the C₆₋₁₀ aryl and 5-10 membered heteroaryl of X² are each optionally substituted with 1, 2, 3, or 4 independently selected R² substituents;
each R² is independently selected from halo, C₁₋₆ alkyl, C₁₋₆ haloalkyl, C₆₋₁₀ aryl, 5-10 membered heteroaryl, CN, NO₂, OR^{a2}, C(O)NR^{c2}R^{d2}, C(O)OR^{a2}, and NR^{c2}R^{d2}, wherein the C₁₋₆ alkyl, C₆₋₁₀ aryl, and 5-10 membered heteroaryl of R² are each optionally substituted with 1, 2, 3, or 4 independently selected R^{2A} substituents;
each R^{a2}, R^{c2}, and R^{d2} is independently selected from H, C₁₋₆ alkyl, and C₆₋₁₀ aryl, wherein the C₁₋₆ alkyl and C₆₋₁₀ aryl of R^{a2}, R^{c2} and R^{d2} are each optionally substituted with 1 or 2 independently selected R^{2A} substituents;
each R^{2A} is independently selected from halo and C₁₋₆ alkyl;
each R⁴ is C₁₋₆ alkyl, wherein the C₁₋₆ alkyl is optionally substituted with 1, 2, 3, or 4 independently selected R^{4A} substituents,
or, alternatively, two R⁴ groups together form a 7-9 membered bridged bicycle with the ring to which the two R⁴ groups are attached; and
each R^{4A} is hydroxy.

In some embodiments:
Ring A is

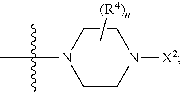

n is 0, 1, or 2;
X² is selected from 5-chloro-2-methylphenyl, 3-nitrophenyl, 2-chloro-6-methylphenyl, 2,4-dichlorophenyl, 2,6-dichlorophenyl, 4-chloro-2-methylphenyl, phenyl, 2-ethylphenyl, 4-fluoro-2-methylphenyl, 5-trifluoromethylphenyl, 3-chloro-2-methylphenyl, (2-methyl)(4-phenyl)phenyl, 2-phenyl-phenyl, 2-cyanophenyl, 2-methoxyphenyl, 2-aminophenyl, naphthalen-1-yl, 3-methoxycarbonyl-6-methyl-phenyl, 3,5-dichloropyridin-2-yl, 3,5-dichloropyridin-4-yl, 3-chloro-5-(trifluoromethyl)pyridin-2-yl, 5-bromo-2-methylphenyl, 4-(4-fluorophenyl)carbamyl-2-chlorophenyl, (1-methyl-1H-pyrazol-4-yl)phenyl, 2-methyl-5-(pyridin-4-yl)phenyl, and 2-methyl-5-(pyridin-3-yl)phenyl;
each R⁴ is C₁₋₆ alkyl, wherein the C₁₋₆ alkyl is optionally substituted with 1, 2, 3, or 4 independently selected R^{4A} substituents,
or, alternatively, two R⁴ groups together form a 7-9 membered bridged bicycle with the ring to which the two R⁴ groups are attached; and
each R^{4A} is hydroxy.

In some embodiments:
Ring A is

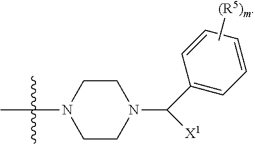

m is 0, 1, or 2;
each R⁵ is independently selected from halo, C₁₋₆ alkyl, C₁₋₆ haloalkyl, C₆₋₁₀ aryl, 5-10 membered heteroaryl, CN, OR^{a5}, C(O)R^{b5}, C(O)NR^{c5}R^{d5}, C(O)OR^{a5}, NR^{c5}R^{d5}, NR^{c5}S(O)₂R^{b5}, and S(O)₂R^{b5}, wherein the C₁₋₆ alkyl, C₆₋₁₀ aryl, and 5-10 membered heteroaryl of R⁵ are each optionally substituted with 1, 2, 3, or 4 independently selected R^{5A} substituents;
   each R^{a5}, R^{b5}, R^{c5}, and R^{d5} is independently selected from H and C₁₋₆ alkyl;
each R^{5A} is independently selected from OR^{a51}, NR^{c51}R^{d51}, NR^{c51}C(O)R^{b51}, NR^{c51}C(O)NR^{c51}R^{d51}, and NR^{c51}S(O)₂R^{b51};
each R^{c51} and R^{d51} is independently selected from H and C₁₋₆ alkyl;
each R^{b51} is independently selected from C₁₋₆ alkyl and 5-6 membered heteroaryl, wherein the 5-6 membered heteroaryl is optionally substituted with 1 or 2 trifluoromethyl groups;
X¹ is selected from C₁₋₆ alkyl, C₆₋₁₀ aryl, C₃₋₁₀ cycloalkyl, 5-10 membered heteroaryl, 4-10 membered heterocycloalkyl, C(O)NHR^{a1}, and C(O)OR^{a1}, wherein the C₁₋₆ alkyl, C₆₋₁₀ aryl, C₃₋₁₀cycloalkyl, 5-10 membered heteroaryl, and 4-10 membered heterocycloalkyl of X¹ are each optionally substituted with 1, 2, 3, or 4 independently selected R¹ substituents;
each R¹ is independently selected from halo, C₁₋₆ alkyl, C₁₋₆ haloalkyl, C₆₋₁₀ aryl, 5-10 membered heteroaryl, CN, OR^{a1}, C(O)R^{b1}, C(O)NR^{c1}R^{d1}, C(O)OR^{a1}, NR^{c1}R^{d1}, NR^{c1}S(O)₂R^{b1}, and S(O)₂R^{b1}, wherein the C₁₋₆ alkyl, C₆₋₁₀ aryl, and 5-10 membered heteroaryl of R¹ are each optionally substituted with 1, 2, 3, or 4 independently selected R^{1A} substituents;
each R^{a1}, R^{c1}, and R^{d1} is independently selected from H, C₁₋₆ alkyl, C₆₋₁₀ aryl, and C₆₋₁₀ aryl-C₁₋₆ alkyl-;
each R^{b1} is independently selected from H and C₁₋₆ alkyl;
each R^{1A} is independently selected from OR^{a11}, NR^{c11}R^{d11}, NR^{c11}C(O)R^{b11}, NR^{c11}C(O)NR^{c11}R^{d11}, and NR^{c11}S(O)₂R^{b11};
each R^{c11} and R^{d11} is independently selected from H and C₁₋₆ alkyl;
each R^{b11} is independently selected from C₁₋₆ alkyl and 5-6 membered heteroaryl, wherein the 5-6 membered heteroaryl is optionally substituted with 1 or 2 independently selected R^{1B} substituents; and
each R^{1B} is independently selected from C₁₋₆ haloalkyl.

In some embodiments:
Ring A is

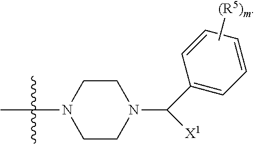

m is 0, 1, or 2;
each R⁵ is independently selected from fluoro, chloro, bromo, trifluoromethyl, CN, methyl, phenyl, methoxy, methylsulfonyl, methoxycarbonyl, carboxy, formyl, hydroxymethyl, carbamyl, tetrazolyl, (trifluoromethylpyrrolyl)sulfonylaminomethyl, aminomethyl, methylcarbamylaminomethyl, pyridinylcarbonylaminomethyl, and methylaminomethyl; and
X¹ is selected from propyl, pentyl, phenyl, cyclopropyl, cyclobutyl, cyclopentyl, cyclohexyl, fluorophenyl, chlorophenyl, bromophenyl, methylphenyl, (trifluoromethyl)phenyl, phenyl-phenyl, (tetrazolyl)phenyl, cyanophenyl, methoxyphenyl, methoxyphenyl, (methylsulfonyl)phenyl, methylcarboxyphenyl, carboxyphenyl, formylphenyl, hydroxymethylphenyl, carbamylphenyl, ((trifluoromethyl)pyrrolesulfonylaminomethyl)phenyl, (aminomethyl)phenyl, (methylaminomethyl)phenyl, methylcarbamylaminomethylphenyl, (pyridinylcarbonylaminomethyl)phenyl, pyridinyl, thiopheneyl, phenylcarbamyl, and benzylcarbamyl.

In some embodiments:
Ring A is

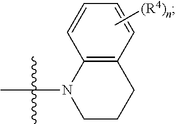

n is 0 or 1;
R⁴ is independently selected from halo, C₆₋₁₀ aryl and 4-10 membered heterocycloalkyl, wherein the C₆₋₁₀ aryl of R⁴ is optionally substituted with 1 or 2 independently selected R^{4A} substituents;
each R^{4A} is independently selected from halo, C₁₋₆ alkyl, C(O)OR^{a41}, and NR^{c41}R^{d41}; and
each R^{a41}, R^{c41}, and R^{d41} is independently selected from H and C₁₋₆ alkyl.

In some embodiments:
Ring A is

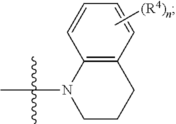

n is 0 or 1;
R⁴ is independently selected from halo, phenyl, and chromanyl, wherein the phenyl of R⁴ is optionally substituted with 1 or 2 independently selected R^{4A} substituents;
each R^{4A} is independently selected from halo, C₁₋₆ alkyl, C(O)OR^{a41}, and NR^{c41}R^{d41}; and
each R^{a41}, R^{c41}, and R^{d41} is independently selected from H and C₁₋₆ alkyl.

In some embodiments:
Ring A is

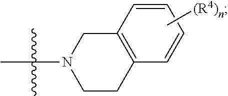

n is 0 or 1; and
R⁴ is independently selected from halo and C₆₋₁₀ aryl.
Ring A is

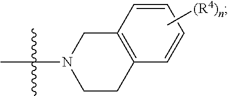

n is 0 or 1; and
R⁴ is independently selected from halo and phenyl.

In some embodiments:
Ring A is

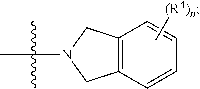

n is 1; and
R⁴ is halo.

In some embodiments:
Ring A is

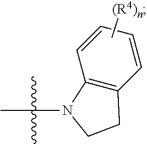

n is 0 or 1; and
R⁴ is halo.

In some embodiments:
Ring A is

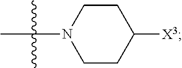

X³ is selected from C₆₋₁₀ aryl and C₆₋₁₀ aryl-C₁₋₆ alkyl-, wherein the C₆₋₁₀ aryl and C₆₋₁₀ aryl-C₁₋₆ alkyl- of X³ are each optionally substituted with 1 or 2 independently selected R³ substituents; and
each R³ is independently selected from halo, C₁₋₆ alkyl, and C₆₋₁₀ aryl.

In some embodiments:
Ring A is

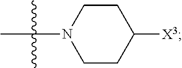

X³ is selected from phenyl and phenyl-C₁₋₆ alkyl-, wherein the phenyl and phenyl-C₁₋₆ alkyl- of X³ are each optionally substituted with 1 or 2 independently selected R³ substituents; and
each R³ is independently selected from halo, C₁₋₆ alkyl, and phenyl.

In some embodiments:
Ring A is

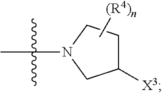

n is 0 or 1;
X³ is C₆₋₁₀ aryl, wherein the C₆₋₁₀ aryl is optionally substituted with 1 or 2 independently selected R³ substituents;
R³ is halo;
R⁴ is independently selected from H and NR^{c4}C(O)OR^{a4}; and
each R^{a4} and R^{c4} is independently selected from H and C₁₋₆ alkyl.

In some embodiments:
Ring A is

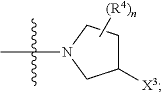

n is 0 or 1;
X³ is phenyl, wherein the C₆₋₁₀ aryl is optionally substituted with 1 or 2 independently selected R³ substituents;
R³ is halo;
R⁴ is independently selected from H and NR^{c4}C(O)OR^{a4}; and
each R^{a4} and R^{c4} is independently selected from H and C₁₋₆ alkyl.

In some embodiments:
Ring A is

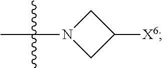

X⁶ is C₆₋₁₀ aryl-C₁₋₆ alkyl-, wherein the C₆₋₁₀ aryl-C₁₋₆ alkyl- is optionally substituted with 1 or 2 independently selected R⁶ substituents; and
each R⁶ is C₆₋₁₀ aryl.

In some embodiments:
Ring A is

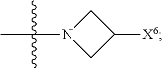

X⁶ is phenyl-C₁₋₆ alkyl-, wherein the phenyl-C₁₋₆ alkyl- is optionally substituted with 1 or 2 independently selected R⁶ substituents; and
each R⁶ is phenyl.

In some embodiments:
Ring A is

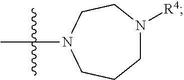

R⁴ is C₆₋₁₀ aryl.

In some embodiments:
Ring A is

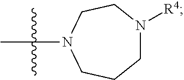

R⁴ is phenyl.

In some embodiments:
Ring A is

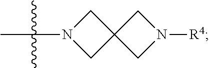

R⁴ is C₆₋₁₀ aryl, wherein the C₆₋₁₀ aryl is optionally substituted with 1 or 2 independently selected R^{4A} substituents; and
each R^{4A} is independently selected from halo and C₁₋₆ alkyl.

In some embodiments:
Ring A is

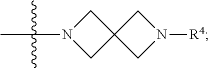

R⁴ is phenyl, wherein the phenyl is optionally substituted with 1 or 2 independently selected R^{4A} substituents; and
each R^{4A} is independently selected from halo and C₁₋₆ alkyl.

In some embodiments:
Ring A is

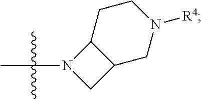

R⁴ is C₆₋₁₀ aryl, wherein the C₆₋₁₀ aryl is optionally substituted with 1 or 2 independently selected R^{4A} substituents; and
each R^{4A} is independently selected from halo and C₁₋₆ alkyl.

In some embodiments:
Ring A is

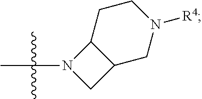

R⁴ is phenyl, wherein the phenyl is optionally substituted with 1 or 2 independently selected R^{4A} substituents; and
each R^{4A} is independently selected from halo and C₁₋₆ alkyl.

In some embodiments:

### Ring A is

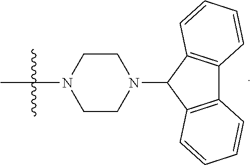 [SMILES: *N1CCN(C2c3ccccc3-c3ccccc32)CC1]

In some embodiments, the compound of Formula I is a compound of Formula II:

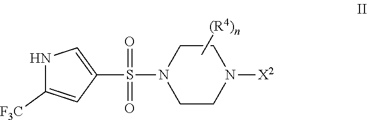

or a pharmaceutically acceptable salt thereof, wherein n is 0, 1, or 2.

In some embodiments, the compound of Formula I is a compound of Formula III:

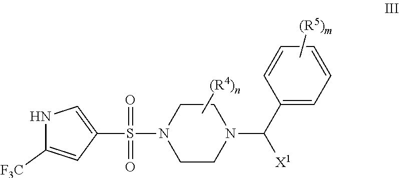

or a pharmaceutically acceptable salt thereof, wherein:
m is 0, 1, or 2; and
n is 0, 1, or 2.

In some embodiments, the compound of Formula I is a compound of Formula IV, Formula V, Formula VI, Formula VII, Formula VIII, Formula IX, Formula X, Formula XI, Formula XII, Formula XIII, or Formula XIV:

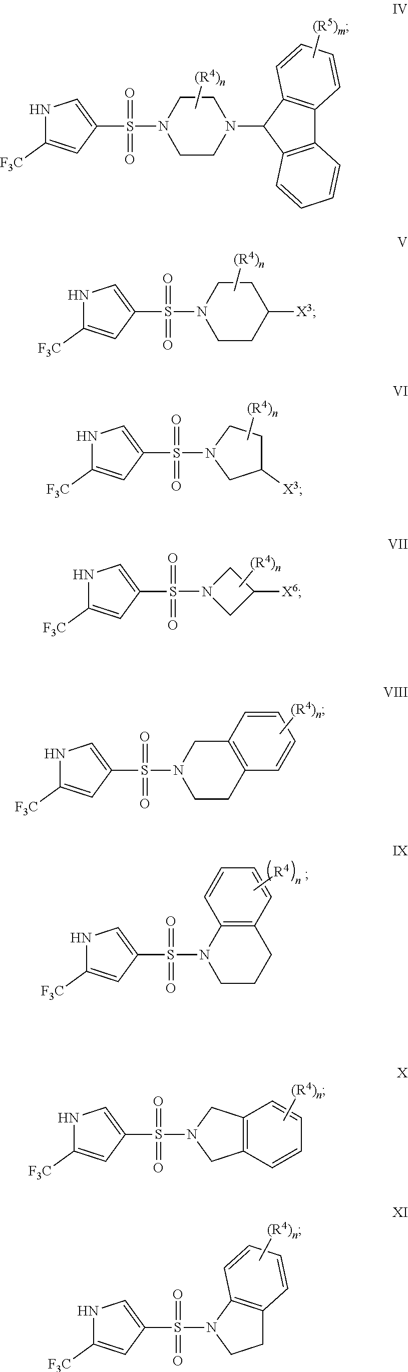

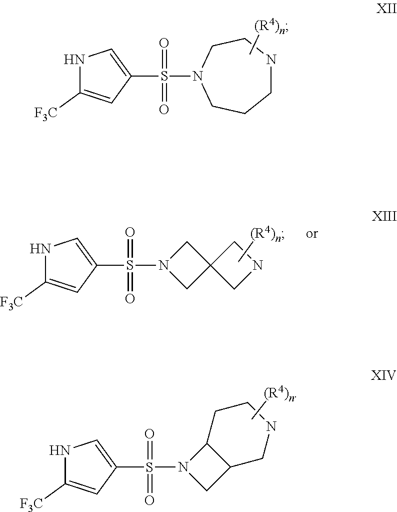

or a pharmaceutically acceptable salt thereof, wherein m is 0, 1, or 2 and n is 0, 1, or 2.

In some embodiments, the compound provided herein is selected from:
1-(5-chloro-2-methylphenyl)-4-((5-(trifluoromethyl)-1H-pyrrol-3-yl)sulfonyl)piperazine;
1-(3-nitrophenyl)-4-((5-(trifluoromethyl)-1H-pyrrol-3-yl)sulfonyl)piperazine;
1-(2-chloro-6-methylphenyl)-4-((5-(trifluoromethyl)-1H-pyrrol-3-yl)sulfonyl)piperazine;
1-(2,4-dichlorophenyl)-4-((5-(trifluoromethyl)-1H-pyrrol-3-yl)sulfonyl)piperazine;
1-(2,6-dichlorophenyl)-4-((5-(trifluoromethyl)-1H-pyrrol-3-yl)sulfonyl)piperazine;
1-(4-chloro-2-methylphenyl)-4-((5-(trifluoromethyl)-1H-pyrrol-3-yl)sulfonyl)piperazine;
1-phenyl-4-((5-(trifluoromethyl)-1H-pyrrol-3-yl)sulfonyl)piperazine;
1-(2-ethylphenyl)-4-((5-(trifluoromethyl)-1H-pyrrol-3-yl)sulfonyl)piperazine;
1-(4-fluoro-2-methylphenyl)-4-((5-(trifluoromethyl)-1H-pyrrol-3-yl)sulfonyl)piperazine;
1-((5-(trifluoromethyl)-1H-pyrrol-3-yl)sulfonyl)-4-(2-(trifluoromethyl)phenyl)piperazine;
1-(3-chloro-2-methylphenyl)-4-((5-(trifluoromethyl)-1H-pyrrol-3-yl)sulfonyl)piperazine;
1-(3-methyl-[1,1′-biphenyl]-4-yl)-4-((5-(trifluoromethyl)-1H-pyrrol-3-yl)sulfonyl)piperazine;
1-([1,1′-biphenyl]-2-yl)-4-((5-(trifluoromethyl)-1H-pyrrol-3-yl)sulfonyl)piperazine;
2-(4-((5-(trifluoromethyl)-1H-pyrrol-3-yl)sulfonyl)piperazin-1-yl)benzonitrile;
1-(2-methoxyphenyl)-4-((5-(trifluoromethyl)-1H-pyrrol-3-yl)sulfonyl)piperazine;
2-(4-((5-(trifluoromethyl)-1H-pyrrol-3-yl)sulfonyl)piperazin-1-yl)aniline;
1-(naphthalen-1-yl)-4-((5-(trifluoromethyl)-1H-pyrrol-3-yl)sulfonyl)piperazine; methyl 4-methyl-3-(4-((5-(trifluoromethyl)-1H-pyrrol-3-yl)sulfonyl)piperazin-1-yl)benzoate;
1-(3,5-dichloropyridin-2-yl)-4-((5-(trifluoromethyl)-1H-pyrrol-3-yl)sulfonyl)piperazine;
1-(3,5-dichloropyridin-4-yl)-4-((5-(trifluoromethyl)-1H-pyrrol-3-yl)sulfonyl)piperazine;
1-(3-chloro-5-(trifluoromethyl)pyridin-2-yl)-4-((5-(trifluoromethyl)-1H-pyrrol-3-yl)sulfonyl)piperazine;
6-bromo-1-((5-(trifluoromethyl)-1H-pyrrol-3-yl)sulfonyl)-1,2,3,4-tetrahydroquinoline;
7-bromo-1-((5-(trifluoromethyl)-1H-pyrrol-3-yl)sulfonyl)-1,2,3,4-tetrahydroquinoline;
5-bromo-1-((5-(trifluoromethyl)-1H-pyrrol-3-yl)sulfonyl)-1,2,3,4-tetrahydroquinoline;
1-((5-(trifluoromethyl)-1H-pyrrol-3-yl)sulfonyl)-1,2,3,4-tetrahydroquinoline;
7-phenyl-2-((5-(trifluoromethyl)-1H-pyrrol-3-yl)sulfonyl)-1,2,3,4-tetrahydroisoquinoline;
5-fluoro-2-((5-(trifluoromethyl)-1H-pyrrol-3-yl)sulfonyl)isoindoline;
7-bromo-1-((5-(trifluoromethyl)-1H-pyrrol-3-yl)sulfonyl)indoline;
1-((5-(trifluoromethyl)-1H-pyrrol-3-yl)sulfonyl)indoline;
4-(2-chlorophenyl)-1-((5-(trifluoromethyl)-1H-pyrrol-3-yl)sulfonyl)piperidine;
4-(5-chloro-2-methylphenyl)-1-((5-(trifluoromethyl)-1H-pyrrol-3-yl)sulfonyl)piperidine;
4-benzhydryl-1-((5-(trifluoromethyl)-1H-pyrrol-3-yl)sulfonyl)piperidine;
4-((3-(2-chlorophenyl)pyrrolidin-1-yl)sulfonyl)-2-(trifluoromethyl)-1H-pyrrole;
tert-butyl (4-(4-fluorophenyl)-1-((5-(trifluoromethyl)-1H-pyrrol-3-yl)sulfonyl)pyrrolidin-3-yl)carbamate;
4-((3-benzhydrylazetidin-1-yl)sulfonyl)-2-(trifluoromethyl)-1H-pyrrole;
1-phenyl-4-((5-(trifluoromethyl)-1H-pyrrol-3-yl)sulfonyl)-1,4-diazepane;
1-(bis(4-fluorophenyl)methyl)-4-((5-(trifluoromethyl)-1H-pyrrol-3-yl)sulfonyl)piperazine;
1-((4-chlorophenyl)(phenyl)methyl)-4-((5-(trifluoromethyl)-1H-pyrrol-3-yl)sulfonyl)piperazine;
1-(bis(4-chlorophenyl)methyl)-4-((5-(trifluoromethyl)-1H-pyrrol-3-yl)sulfonyl)piperazine;
1-((2-chlorophenyl)(phenyl)methyl)-4-((5-(trifluoromethyl)-1H-pyrrol-3-yl)sulfonyl)piperazine;
1-(phenyl(pyridin-2-yl)methyl)-4-((5-(trifluoromethyl)-1H-pyrrol-3-yl)sulfonyl)piperazine;
1-(5-bromo-2-methylphenyl)-4-((5-(trifluoromethyl)-1H-pyrrol-3-yl)sulfonyl)piperazine;
6-bromo-2-((5-(trifluoromethyl)-1H-pyrrol-3-yl)sulfonyl)-1,2,3,4-tetrahydroisoquinoline;
7-bromo-2-((5-(trifluoromethyl)-1H-pyrrol-3-yl)sulfonyl)-1,2,3,4-tetrahydroisoquinoline;
3-chloro-N-(4-fluorophenyl)-4-(4-((5-(trifluoromethyl)-1H-pyrrol-3-yl)sulfonyl)piperazin-1-yl)benzamide;
1-(2-(1-methyl-1H-pyrazol-4-yl)phenyl)-4-((5-(trifluoromethyl)-1H-pyrrol-3-yl)sulfonyl)piperazine;
1-(2-methyl-5-(pyridin-4-yl)phenyl)-4-((5-(trifluoromethyl)-1H-pyrrol-3-yl)sulfonyl)piperazine;
5-(2-fluoro-4-methylphenyl)-1-((5-(trifluoromethyl)-1H-pyrrol-3-yl)sulfonyl)-1,2,3,4-tetrahydroquinoline;
N,N-dimethyl-4-(1-((5-(trifluoromethyl)-1H-pyrrol-3-yl)sulfonyl)-1,2,3,4-tetrahydroquinolin-5-yl)aniline;
5-(chroman-6-yl)-1-((5-(trifluoromethyl)-1H-pyrrol-3-yl)sulfonyl)-1,2,3,4-tetrahydroquinoline;
5-(o-tolyl)-1-((5-(trifluoromethyl)-1H-pyrrol-3-yl)sulfonyl)-1,2,3,4-tetrahydroquinoline;
methyl 3-(1-((5-(trifluoromethyl)-1H-pyrrol-3-yl)sulfonyl)-1,2,3,4-tetrahydroquinolin-5-yl)benzoate;
6-phenyl-2-((5-(trifluoromethyl)-1H-pyrrol-3-yl)sulfonyl)-1,2,3,4-tetrahydroisoquinoline;
4-(5-chloro-2-methylphenyl)-2-methyl-1-((5-(trifluoromethyl)-1H-pyrrol-3-yl)sulfonyl)piperazine;
1-(5-chloro-2-methylphenyl)-2-methyl-4-((5-(trifluoromethyl)-1H-pyrrol-3-yl)sulfonyl)piperazine;
4-(5-chloro-2-methylphenyl)-2,6-dimethyl-1-((5-(trifluoromethyl)-1H-pyrrol-3-yl)sulfonyl)piperazine;
(4-(5-chloro-2-methylphenyl)-1-((5-(trifluoromethyl)-1H-pyrrol-3-yl)sulfonyl)piperazin-2-yl)methanol;
2-(4-chloro-2-methylphenyl)-5-((5-(trifluoromethyl)-1H-pyrrol-3-yl)sulfonyl)-2,5-diazabicyclo[2.2.1]heptane;
2-(5-chloro-2-methylphenyl)-6-((5-(trifluoromethyl)-1H-pyrrol-3-yl)sulfonyl)-2,6-diazaspiro[3.3]heptane;
3-(5-chloro-2-methylphenyl)-7-((5-(trifluoromethyl)-1H-pyrrol-3-yl)sulfonyl)-3,7-diazabicyclo[4.2.0]octane;
(S)-2-methyl-4-(2-methyl-5-(pyridin-3-yl)phenyl)-1-((5-(trifluoromethyl)-1H-pyrrol-3-yl)sulfonyl)piperazine;
4-(phenyl(4-((5-(trifluoromethyl)-1H-pyrrol-3-yl)sulfonyl)piperazin-1-yl)methyl)benzonitrile;
1-(bis(4-bromophenyl)methyl)-4-((5-(trifluoromethyl)-1H-pyrrol-3-yl)sulfonyl)piperazine;
4-((4-chlorophenyl)(4-((5-(trifluoromethyl)-1H-pyrrol-3-yl)sulfonyl)piperazin-1-yl)methyl)benzonitrile;
1-((4-chlorophenyl)(4-fluorophenyl)methyl)-4-((5-(trifluoromethyl)-1H-pyrrol-3-yl)sulfonyl)piperazine;
1-((4-bromophenyl)(phenyl)methyl)-4-((5-(trifluoromethyl)-1H-pyrrol-3-yl)sulfonyl)piperazine;
4,4′-((4-((5-(trifluoromethyl)-1H-pyrrol-3-yl)sulfonyl)piperazin-1-yl)methylene)dibenzonitrile;
1-((4-fluorophenyl)(phenyl)methyl)-4-((5-(trifluoromethyl)-1H-pyrrol-3-yl)sulfonyl)piperazine;
1-((2-bromophenyl)(phenyl)methyl)-4-((5-(trifluoromethyl)-1H-pyrrol-3-yl)sulfonyl)piperazine;
1-((4-chlorophenyl)(4-(trifluoromethyl)phenyl)methyl)-4-((5-(trifluoromethyl)-1H-pyrrol-3-yl)sulfonyl)piperazine;
1-((2-fluorophenyl)(phenyl)methyl)-4-((5-(trifluoromethyl)-1H-pyrrol-3-yl)sulfonyl)piperazine;
1-((4-fluorophenyl)(4-methoxyphenyl)methyl)-4-((5-(trifluoromethyl)-1H-pyrrol-3-yl)sulfonyl)piperazine;
1-(di-p-tolylmethyl)-4-((5-(trifluoromethyl)-1H-pyrrol-3-yl)sulfonyl)piperazine;
1-([1,1′-biphenyl]-4-yl(phenyl)methyl)-4-((5-(trifluoromethyl)-1H-pyrrol-3-yl)sulfonyl)piperazine;
1-(phenyl(o-tolyl)methyl)-4-((5-(trifluoromethyl)-1H-pyrrol-3-yl)sulfonyl)piperazine;
1-(di-o-tolylmethyl)-4-((5-(trifluoromethyl)-1H-pyrrol-3-yl)sulfonyl)piperazine;
1-(bis(3-methoxyphenyl)methyl)-4-((5-(trifluoromethyl)-1H-pyrrol-3-yl)sulfonyl)piperazine;
1-((3-bromophenyl)(phenyl)methyl)-4-((5-(trifluoromethyl)-1H-pyrrol-3-yl)sulfonyl)piperazine;
1-((4-(methylsulfonyl)phenyl)(phenyl)methyl)-4-((5-(trifluoromethyl)-1H-pyrrol-3-yl)sulfonyl)piperazine;
1-(phenyl(thiophen-2-yl)methyl)-4-((5-(trifluoromethyl)-1H-pyrrol-3-yl)sulfonyl)piperazine;
1-(9H-fluoren-9-yl)-4-((5-(trifluoromethyl)-1H-pyrrol-3-yl)sulfonyl)piperazine;
methyl 4-(phenyl(4-((5-(trifluoromethyl)-1H-pyrrol-3-yl)sulfonyl)piperazin-1-yl)methyl)benzoate;
4-(phenyl(4-((5-(trifluoromethyl)-1H-pyrrol-3-yl)sulfonyl)piperazin-1-yl)methyl)benzoic acid;
1-(cyclopentyl(phenyl)methyl)-4-((5-(trifluoromethyl)-1H-pyrrol-3-yl)sulfonyl)piperazine;
1-(2-ethyl-1-phenylbutyl)-4-((5-(trifluoromethyl)-1H-pyrrol-3-yl)sulfonyl)piperazine;
1-((4-chlorophenyl)(cyclobutyl)methyl)-4-((5-(trifluoromethyl)-1H-pyrrol-3-yl)sulfonyl)piperazine;
1-((4-chlorophenyl)(cyclohexyl)methyl)-4-((5-(trifluoromethyl)-1H-pyrrol-3-yl)sulfonyl)piperazine;
1-(2-methyl-1-phenylpropyl)-4-((5-(trifluoromethyl)-1H-pyrrol-3-yl)sulfonyl)piperazine;
1-(cyclopropyl(4-(trifluoromethyl)phenyl)methyl)-4-((5-(trifluoromethyl)-1H-pyrrol-3-yl)sulfonyl)piperazine;
N,2-diphenyl-2-(4-((5-(trifluoromethyl)-1H-pyrrol-3-yl)sulfonyl)piperazin-1-yl)acetamide;
N-benzyl-2-phenyl-2-(4-((5-(trifluoromethyl)-1H-pyrrol-3-yl)sulfonyl)piperazin-1-yl)acetamide;
4-(phenyl(4-((5-(trifluoromethyl)-1H-pyrrol-3-yl)sulfonyl)piperazin-1-yl)methyl)benzaldehyde;
(4-(phenyl(4-((5-(trifluoromethyl)-1H-pyrrol-3-yl)sulfonyl)piperazin-1-yl)methyl)phenyl)methanol;
4-(phenyl(4-((5-(trifluoromethyl)-1H-pyrrol-3-yl)sulfonyl)piperazin-1-yl)methyl)benzamide;
1-((4-(1H-tetrazol-5-yl)phenyl)(phenyl)methyl)-4-((5-(trifluoromethyl)-1H-pyrrol-3-yl)sulfonyl)piperazine;
N-(4-(phenyl(4-((5-(trifluoromethyl)-1H-pyrrol-3-yl)sulfonyl)piperazin-1-yl)methyl)benzyl)-5-(trifluoromethyl)-1H-pyrrole-3-sulfonamide;
(4-(phenyl(4-((5-(trifluoromethyl)-1H-pyrrol-3-yl)sulfonyl)piperazin-1-yl)methyl)phenyl)methanamine;
1-methyl-3-(4-(phenyl(4-((5-(trifluoromethyl)-1H-pyrrol-3-yl)sulfonyl)piperazin-1-yl)methyl)benzyl)urea;
N-(4-(phenyl(4-((5-(trifluoromethyl)-1H-pyrrol-3-yl)sulfonyl)piperazin-1-yl)methyl)benzyl)isonicotinamide; and
N-methyl-1-(4-(phenyl(4-((5-(trifluoromethyl)-1H-pyrrol-3-yl)sulfonyl)piperazin-1-yl)methyl)phenyl)methanamine;

or a pharmaceutically acceptable salt thereof.

In some embodiments, the compound provided herein is selected from:
1-(5-chloro-2-methylphenyl)-4-((5-(trifluoromethyl)-1H-pyrrol-3-yl)sulfonyl)piperazine;
1-(3-nitrophenyl)-4-((5-(trifluoromethyl)-1H-pyrrol-3-yl)sulfonyl)piperazine;
1-(2-chloro-6-methylphenyl)-4-((5-(trifluoromethyl)-1H-pyrrol-3-yl)sulfonyl)piperazine;
1-(2,4-dichlorophenyl)-4-((5-(trifluoromethyl)-1H-pyrrol-3-yl)sulfonyl)piperazine;
1-(2,6-dichlorophenyl)-4-((5-(trifluoromethyl)-1H-pyrrol-3-yl)sulfonyl)piperazine;
1-(4-chloro-2-methylphenyl)-4-((5-(trifluoromethyl)-1H-pyrrol-3-yl)sulfonyl)piperazine;
1-phenyl-4-((5-(trifluoromethyl)-1H-pyrrol-3-yl)sulfonyl)piperazine;
1-(2-ethylphenyl)-4-((5-(trifluoromethyl)-1H-pyrrol-3-yl)sulfonyl)piperazine;
1-(4-fluoro-2-methylphenyl)-4-((5-(trifluoromethyl)-1H-pyrrol-3-yl)sulfonyl)piperazine;
1-((5-(trifluoromethyl)-1H-pyrrol-3-yl)sulfonyl)-4-(2-(trifluoromethyl)phenyl)piperazine;
1-(3-chloro-2-methylphenyl)-4-((5-(trifluoromethyl)-1H-pyrrol-3-yl)sulfonyl)piperazine;
1-(3-methyl-[1,1′-biphenyl]-4-yl)-4-((5-(trifluoromethyl)-1H-pyrrol-3-yl)sulfonyl)piperazine;
1-([1,1′-biphenyl]-2-yl)-4-((5-(trifluoromethyl)-1H-pyrrol-3-yl)sulfonyl)piperazine;
2-(4-((5-(trifluoromethyl)-1H-pyrrol-3-yl)sulfonyl)piperazin-1-yl)benzonitrile;
1-(2-methoxyphenyl)-4-((5-(trifluoromethyl)-1H-pyrrol-3-yl)sulfonyl)piperazine;
2-(4-((5-(trifluoromethyl)-1H-pyrrol-3-yl)sulfonyl)piperazin-1-yl)aniline;
1-(naphthalen-1-yl)-4-((5-(trifluoromethyl)-1H-pyrrol-3-yl)sulfonyl)piperazine;
methyl 4-methyl-3-(4-((5-(trifluoromethyl)-1H-pyrrol-3-yl)sulfonyl)piperazin-1-yl)benzoate;
1-(3,5-dichloropyridin-2-yl)-4-((5-(trifluoromethyl)-1H-pyrrol-3-yl)sulfonyl)piperazine;
1-(3,5-dichloropyridin-4-yl)-4-((5-(trifluoromethyl)-1H-pyrrol-3-yl)sulfonyl)piperazine;
1-(3-chloro-5-(trifluoromethyl)pyridin-2-yl)-4-((5-(trifluoromethyl)-1H-pyrrol-3-yl)sulfonyl)piperazine;
6-bromo-1-((5-(trifluoromethyl)-1H-pyrrol-3-yl)sulfonyl)-1,2,3,4-tetrahydroquinoline;
7-bromo-1-((5-(trifluoromethyl)-1H-pyrrol-3-yl)sulfonyl)-1,2,3,4-tetrahydroquinoline;
5-bromo-1-((5-(trifluoromethyl)-1H-pyrrol-3-yl)sulfonyl)-1,2,3,4-tetrahydroquinoline;
1-((5-(trifluoromethyl)-1H-pyrrol-3-yl)sulfonyl)-1,2,3,4-tetrahydroquinoline;
7-phenyl-2-((5-(trifluoromethyl)-1H-pyrrol-3-yl)sulfonyl)-1,2,3,4-tetrahydroisoquinoline;
5-fluoro-2-((5-(trifluoromethyl)-1H-pyrrol-3-yl)sulfonyl)isoindoline;
7-bromo-1-((5-(trifluoromethyl)-1H-pyrrol-3-yl)sulfonyl)indoline;
1-((5-(trifluoromethyl)-1H-pyrrol-3-yl)sulfonyl)indoline;
4-(2-chlorophenyl)-1-((5-(trifluoromethyl)-1H-pyrrol-3-yl)sulfonyl)piperidine;
4-(5-chloro-2-methylphenyl)-1-((5-(trifluoromethyl)-1H-pyrrol-3-yl)sulfonyl)piperidine;
4-benzhydryl-1-((5-(trifluoromethyl)-1H-pyrrol-3-yl)sulfonyl)piperidine;
4-((3-(2-chlorophenyl)pyrrolidin-1-yl)sulfonyl)-2-(trifluoromethyl)-1H-pyrrole;
trans-tert-butyl (4-(4-fluorophenyl)-1-((5-(trifluoromethyl)-1H-pyrrol-3-yl)sulfonyl)pyrrolidin-3-yl)carbamate;
4-((3-benzhydrylazetidin-1-yl)sulfonyl)-2-(trifluoromethyl)-1H-pyrrole;
1-phenyl-4-((5-(trifluoromethyl)-1H-pyrrol-3-yl)sulfonyl)-1,4-diazepane;
1-(bis(4-fluorophenyl)methyl)-4-((5-(trifluoromethyl)-1H-pyrrol-3-yl)sulfonyl)piperazine;
1-((4-chlorophenyl)(phenyl)methyl)-4-((5-(trifluoromethyl)-1H-pyrrol-3-yl)sulfonyl)piperazine;
1-(bis(4-chlorophenyl)methyl)-4-((5-(trifluoromethyl)-1H-pyrrol-3-yl)sulfonyl)piperazine;
1-((2-chlorophenyl)(phenyl)methyl)-4-((5-(trifluoromethyl)-1H-pyrrol-3-yl)sulfonyl)piperazine;
1-(phenyl(pyridin-2-yl)methyl)-4-((5-(trifluoromethyl)-1H-pyrrol-3-yl)sulfonyl)piperazine;
1-(5-bromo-2-methylphenyl)-4-((5-(trifluoromethyl)-1H-pyrrol-3-yl)sulfonyl)piperazine;
6-bromo-2-((5-(trifluoromethyl)-1H-pyrrol-3-yl)sulfonyl)-1,2,3,4-tetrahydroisoquinoline;
7-bromo-2-((5-(trifluoromethyl)-1H-pyrrol-3-yl)sulfonyl)-1,2,3,4-tetrahydroisoquinoline;
3-chloro-N-(4-fluorophenyl)-4-(4-((5-(trifluoromethyl)-1H-pyrrol-3-yl)sulfonyl)piperazin-1-yl)benzamide;
1-(2-(1-methyl-1H-pyrazol-4-yl)phenyl)-4-((5-(trifluoromethyl)-1H-pyrrol-3-yl)sulfonyl)piperazine;
1-(2-methyl-5-(pyridin-4-yl)phenyl)-4-((5-(trifluoromethyl)-1H-pyrrol-3-yl)sulfonyl)piperazine;
5-(2-fluoro-4-methylphenyl)-1-((5-(trifluoromethyl)-1H-pyrrol-3-yl)sulfonyl)-1,2,3,4-tetrahydroquinoline;
N,N-dimethyl-4-(1-((5-(trifluoromethyl)-1H-pyrrol-3-yl)sulfonyl)-1,2,3,4-tetrahydroquinolin-5-yl)aniline;
5-(chroman-6-yl)-1-((5-(trifluoromethyl)-1H-pyrrol-3-yl)sulfonyl)-1,2,3,4-tetrahydroquinoline;
5-(o-tolyl)-1-((5-(trifluoromethyl)-1H-pyrrol-3-yl)sulfonyl)-1,2,3,4-tetrahydroquinoline;
methyl 3-(1-((5-(trifluoromethyl)-1H-pyrrol-3-yl)sulfonyl)-1,2,3,4-tetrahydroquinolin-5-yl)benzoate;
6-phenyl-2-((5-(trifluoromethyl)-1H-pyrrol-3-yl)sulfonyl)-1,2,3,4-tetrahydroisoquinoline;
(R)-4-(5-chloro-2-methylphenyl)-2-methyl-1-((5-(trifluoromethyl)-1H-pyrrol-3-yl)sulfonyl)piperazine;
(S)-4-(5-chloro-2-methylphenyl)-2-methyl-1-((5-(trifluoromethyl)-1H-pyrrol-3-yl)sulfonyl)piperazine;
1-(5-chloro-2-methylphenyl)-2-methyl-4-((5-(trifluoromethyl)-1H-pyrrol-3-yl)sulfonyl)piperazine;
4-(5-chloro-2-methylphenyl)-2,6-dimethyl-1-((5-(trifluoromethyl)-1H-pyrrol-3-yl)sulfonyl)piperazine;
(4-(5-chloro-2-methylphenyl)-1-((5-(trifluoromethyl)-1H-pyrrol-3-yl)sulfonyl)piperazin-2-yl)methanol;
(1S,4S)-2-(4-chloro-2-methylphenyl)-5-((5-(trifluoromethyl)-1H-pyrrol-3-yl)sulfonyl)-2,5-diazabicyclo[2.2.1]heptane;
2-(5-chloro-2-methylphenyl)-6-((5-(trifluoromethyl)-1H-pyrrol-3-yl)sulfonyl)-2,6-diazaspiro[3.3]heptane;
3-(5-chloro-2-methylphenyl)-7-((5-(trifluoromethyl)-1H-pyrrol-3-yl)sulfonyl)-3,7-diazabicyclo[4.2.0]octane;
(S)-2-methyl-4-(2-methyl-5-(pyridin-3-yl)phenyl)-1-((5-(trifluoromethyl)-1H-pyrrol-3-yl)sulfonyl)piperazine;
4-(phenyl(4-((5-(trifluoromethyl)-1H-pyrrol-3-yl)sulfonyl)piperazin-1-yl)methyl)benzonitrile;
1-(bis(4-bromophenyl)methyl)-4-((5-(trifluoromethyl)-1H-pyrrol-3-yl)sulfonyl)piperazine;
4-((4-chlorophenyl)(4-((5-(trifluoromethyl)-1H-pyrrol-3-yl)sulfonyl)piperazin-1-yl)methyl)benzonitrile;
1-((4-chlorophenyl)(4-fluorophenyl)methyl)-4-((5-(trifluoromethyl)-1H-pyrrol-3-yl)sulfonyl)piperazine;
1-((4-bromophenyl)(phenyl)methyl)-4-((5-(trifluoromethyl)-1H-pyrrol-3-yl)sulfonyl)piperazine;
4,4′-((4-((5-(trifluoromethyl)-1H-pyrrol-3-yl)sulfonyl)piperazin-1-yl)methylene)dibenzonitrile;
1-((4-fluorophenyl)(phenyl)methyl)-4-((5-(trifluoromethyl)-1H-pyrrol-3-yl)sulfonyl)piperazine;
1-((2-bromophenyl)(phenyl)methyl)-4-((5-(trifluoromethyl)-1H-pyrrol-3-yl)sulfonyl)piperazine;
1-((4-chlorophenyl)(4-(trifluoromethyl)phenyl)methyl)-4-((5-(trifluoromethyl)-1H-pyrrol-3-yl)sulfonyl)piperazine;
1-((2-fluorophenyl)(phenyl)methyl)-4-((5-(trifluoromethyl)-1H-pyrrol-3-yl)sulfonyl)piperazine;
1-((4-fluorophenyl)(4-methoxyphenyl)methyl)-4-((5-(trifluoromethyl)-1H-pyrrol-3-yl)sulfonyl)piperazine;
1-(di-p-tolylmethyl)-4-((5-(trifluoromethyl)-1H-pyrrol-3-yl)sulfonyl)piperazine;
1-([1,1′-biphenyl]-4-yl(phenyl)methyl)-4-((5-(trifluoromethyl)-1H-pyrrol-3-yl)sulfonyl)piperazine;
1-(phenyl(o-tolyl)methyl)-4-((5-(trifluoromethyl)-1H-pyrrol-3-yl)sulfonyl)piperazine;
1-(di-o-tolylmethyl)-4-((5-(trifluoromethyl)-1H-pyrrol-3-yl)sulfonyl)piperazine;
1-(bis(3-methoxyphenyl)methyl)-4-((5-(trifluoromethyl)-1H-pyrrol-3-yl)sulfonyl)piperazine;
1-((3-bromophenyl)(phenyl)methyl)-4-((5-(trifluoromethyl)-1H-pyrrol-3-yl)sulfonyl)piperazine;
1-((4-(methylsulfonyl)phenyl)(phenyl)methyl)-4-((5-(trifluoromethyl)-1H-pyrrol-3-yl)sulfonyl)piperazine;
1-(phenyl(thiophen-2-yl)methyl)-4-((5-(trifluoromethyl)-1H-pyrrol-3-yl)sulfonyl)piperazine;
1-(9H-fluoren-9-yl)-4-((5-(trifluoromethyl)-1H-pyrrol-3-yl)sulfonyl)piperazine;
methyl 4-(phenyl(4-((5-(trifluoromethyl)-1H-pyrrol-3-yl)sulfonyl)piperazin-1-yl)methyl)benzoate;
4-(phenyl(4-((5-(trifluoromethyl)-1H-pyrrol-3-yl)sulfonyl)piperazin-1-yl)methyl)benzoic acid;
1-(cyclopentyl(phenyl)methyl)-4-((5-(trifluoromethyl)-1H-pyrrol-3-yl)sulfonyl)piperazine;
1-(2-ethyl-1-phenylbutyl)-4-((5-(trifluoromethyl)-1H-pyrrol-3-yl)sulfonyl)piperazine;
1-((4-chlorophenyl)(cyclobutyl)methyl)-4-((5-(trifluoromethyl)-1H-pyrrol-3-yl)sulfonyl)piperazine;
1-((4-chlorophenyl)(cyclohexyl)methyl)-4-((5-(trifluoromethyl)-1H-pyrrol-3-yl)sulfonyl)piperazine;
1-(2-methyl-1-phenylpropyl)-4-((5-(trifluoromethyl)-1H-pyrrol-3-yl)sulfonyl)piperazine;
1-(cyclopropyl(4-(trifluoromethyl)phenyl)methyl)-4-((5-(trifluoromethyl)-1H-pyrrol-3-yl)sulfonyl)piperazine;
N,2-diphenyl-2-(4-((5-(trifluoromethyl)-1H-pyrrol-3-yl)sulfonyl)piperazin-1-yl)acetamide;
N-benzyl-2-phenyl-2-(4-((5-(trifluoromethyl)-1H-pyrrol-3-yl)sulfonyl)piperazin-1-yl)acetamide;
4-(phenyl(4-((5-(trifluoromethyl)-1H-pyrrol-3-yl)sulfonyl)piperazin-1-yl)methyl)benzaldehyde;
(4-(phenyl(4-((5-(trifluoromethyl)-1H-pyrrol-3-yl)sulfonyl)piperazin-1-yl)methyl)phenyl)methanol;
4-(phenyl(4-((5-(trifluoromethyl)-1H-pyrrol-3-yl)sulfonyl)piperazin-1-yl)methyl)benzamide;
1-((4-(1H-tetrazol-5-yl)phenyl)(phenyl)methyl)-4-((5-(trifluoromethyl)-1H-pyrrol-3-yl)sulfonyl)piperazine;
N-(4-(phenyl(4-((5-(trifluoromethyl)-1H-pyrrol-3-yl)sulfonyl)piperazin-1-yl)methyl)benzyl)-5-(trifluoromethyl)-1H-pyrrole-3-sulfonamide;
(4-(phenyl(4-((5-(trifluoromethyl)-1H-pyrrol-3-yl)sulfonyl)piperazin-1-yl)methyl)phenyl)methanamine;
1-methyl-3-(4-(phenyl(4-((5-(trifluoromethyl)-1H-pyrrol-3-yl)sulfonyl)piperazin-1-yl)methyl)benzyl)urea;
N-(4-(phenyl(4-((5-(trifluoromethyl)-1H-pyrrol-3-yl)sulfonyl)piperazin-1-yl)methyl)benzyl)isonicotinamide; and
N-methyl-1-(4-(phenyl(4-((5-(trifluoromethyl)-1H-pyrrol-3-yl)sulfonyl)piperazin-1-yl)methyl)phenyl)methanamine;

or a pharmaceutically acceptable salt thereof.

In some embodiments, the compound is 4-(phenyl(4-((5-(trifluoromethyl)-1H-pyrrol-3-yl)sulfonyl)piperazin-1-yl)methyl)benzonitrile, or a pharmaceutically acceptable salt thereof.

In some embodiments, the compound is 4-((4-chlorophenyl)(4-((5-(trifluoromethyl)-1H-pyrrol-3-yl)sulfonyl)piperazin-1-yl)methyl)benzonitrile, or a pharmaceutically acceptable salt thereof.

In some embodiments, the compound is 4-(phenyl(4-((5-(trifluoromethyl)-1H-pyrrol-3-yl)sulfonyl)piperazin-1-yl)methyl)benzonitrile.

In some embodiments, the compound is 4-((4-chlorophenyl)(4-((5-(trifluoromethyl)-1H-pyrrol-3-yl)sulfonyl)piperazin-1-yl)methyl)benzonitrile.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described.

It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “Cₙ₋ₘ alkyl” is understood to include deuterated analogs of alkyl groups as defined herein, including but not limited to, groups such as deuteromethyl (CDH₂), dideuteromethyl (CD₂H), trideuteromethyl (CD₃), pentadeuteroethyl (CD₂CD₃), heptadeuteroisopropyl (CD(CD₃)₂), and the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenyl” is understood to include deuterated analogs of alkenyl groups as defined herein, including but not limited to, groups such as trideuteroethenyl (—CD═CD₂), tetradeuteropropenyl, (—CD═CD-CD₂), and the like.

As used herein, “Cₙ₋ₘ alkynyl” refers to an alkyl group having one or more triple carbon-carbon bonds and having n to m carbons. Example alkynyl groups include, but are not limited to, ethynyl, propynyl (e.g., propyn-1-yl, propyn-2-yl, prop-2-yn-1-yl), and the like. In some embodiments, the alkynyl moiety contains 2 to 6, 2 to 4, or 2 to 3 carbon atoms. The term “Cₙ₋ₘ alkynyl” is understood to include deuterated analogs of alkynyl groups as defined herein, including but not limited to, groups such as deuteroethynyl (—C≡CD), trideuteropropyn-1-yl, (—C≡CCD₃), and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy group as defined herein, including but not limited to, groups such as trideuteromethoxy (—OCD₃), pentadeuteroethoxy (—OCD₂CD₃), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-d₅), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F or Br. In some embodiments, a halo is Cl or Br. In some embodiments, a halo is F. In some embodiments, a halo is Cl. In some embodiments, a halo is Br.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms. The term “Cₙ₋ₘ haloalkoxy” is understood to include deuterated analogs of the haloalkyl moiety of the haloalkoxy group as defined herein, including but not limited to, groups such as deuterodifluoromethoxy (—OCDF₂), dideuterofluoromethoxy (—OCD₂F), and the like.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl group as defined herein, including but not limited to, groups such as deuterodifluoromethyl (—CDF₂), dideuterofluoromethyl (—CD₂F), and the like.

As used herein, the term “carbonyl”, employed alone or in combination with other terms, refers to a —C(O)— group.

As used herein, the term “sulfonyl”, employed alone or in combination with other terms, refers to a —S(O)₂— group.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carboxy” refers to a group of formula —C(O)OH.

As used herein, the term “formyl” refers to a group of formula —C(O)H.

As used herein, the term “carbamyl” refers to a group of formula —C(O)NH₂.

As used herein, the term “di(Cₙ₋ₘ alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, perdeuterocyclopentyl, perdeuterocyclohexyl, and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1, 2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, imidazo[1,2-b]pyridazinyl, pyrazolo[1,5-a]pyrimidinyl, and the like. The term “heteroaryl” is understood to include deuterated analogs of the heteroaryl groups as defined herein, including but not limited to, groups such as perdeuteropyridinyl, perdeuteropyrazinyl, perdeuteropyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula Ia, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula I, such as compounds of Formula I′, can be prepared as shown in Scheme 1. Sulfonylation of cyclic amines 1-1 with sulfonyl chloride 1-2 in the presence of a base such as triethylamine or N,N-diisopropylethylamine can afford compounds of Formula I′. Alternatively, protected cyclic amines 1-3 having an appropriate N-protecting group (e.g., Boc) can be deprotected under suitable reaction conditions (e.g., with acid such as trifluoroacetic acid for the removal of Boc) to provide the amine 1-1 which can then be reacted with sulfonyl chloride 1-2 to provide compounds of Formula I′.

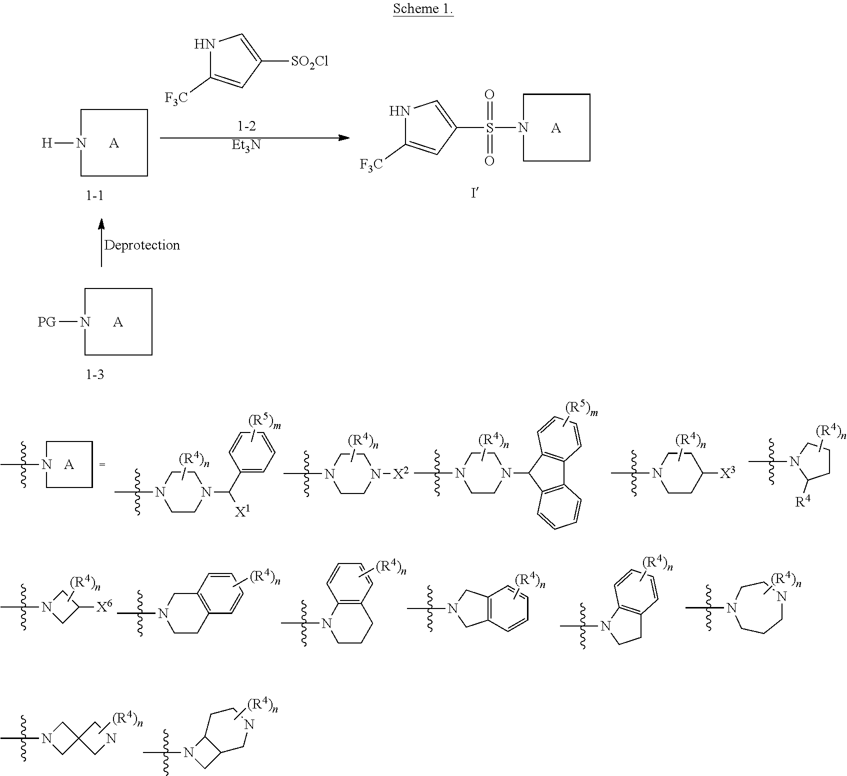

Compounds of Formula I, such as compounds of Formula Ia and Ib, can also be prepared as shown in Scheme 2. Suitable starting materials 2-1, which can be synthesized through Scheme 1, where R¹ is an ester can be hydrolyzed under aqueous basic conditions (e.g., aqueous lithium hydroxide) to deliver carboxylic acid 2-2. The acid can be converted to compounds of Formula Ia by standard amide formation conditions (e.g., in the presence of amine 2-3, a coupling reagent, such as hexafluorophosphate azabenzotriazole uronium [HATU] and a base, such as triethylamine). Examples where R¹ is a halogen (e.g., Cl, Br, or I) as in 2-4, can be coupled with R⁶-M¹ (2-5), where M¹ is a boronic acid, boronate ester, potassium trifluoroborate, or an appropriately substituted metal such as Sn(Bu)₃ or Zn, under standard Suzuki conditions (e.g., in the presence of a palladium catalyst, such as chloro(2-dicyclohexylphosphino-2′,4′,6′-triisopropyl-1,1′-biphenyl)[2-(2′-amino-1,1′-biphenyl)]palladium(II) and a base (e.g., a phosphate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compounds of Formula Ib.

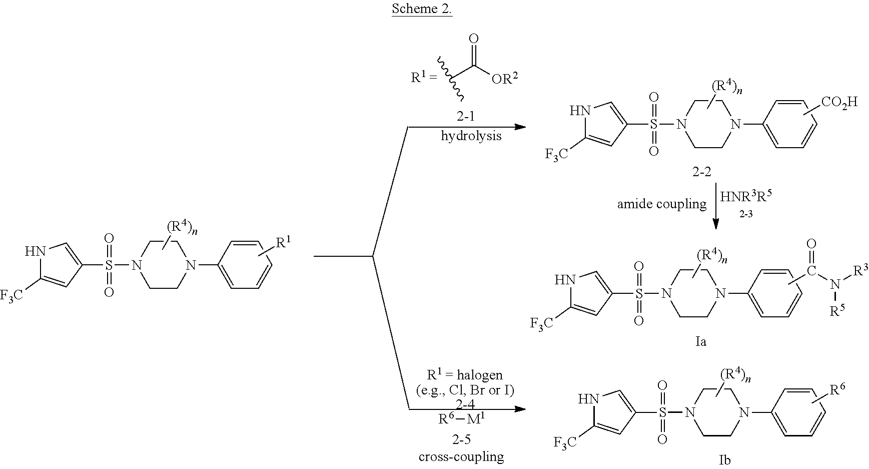

Compounds of Formula I, such as compounds of Formula Ic and Id, can also be prepared as shown in Scheme 3. Protected amines of Formula 3-1 and Formula 3-2 having an appropriate N-protecting group (e.g., Boc) and where R¹ is a halogen (e.g., Cl, Br, or I) can be coupled with R⁴-M¹ (3-3), where M¹ is a boronic acid, boronate ester, potassium trifluoroborate, or an appropriately substituted metal such as Sn(Bu)₃ or Zn, under standard Suzuki conditions (e.g., in the presence of a palladium catalyst, such as chloro(2-dicyclohexylphosphino-2′,4′,6′-triisopropyl-1,1′-biphenyl)[2-(2′-amino-1,1′-biphenyl)]palladium(II) and a base (e.g., a phosphate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)) to provide compounds of Formula 3-4 and Formula 3-5. Deprotection under suitable reaction conditions (e.g., with acid such as trifluoroacetic acid for the removal of Boc) can provide amines of Formula 3-6 and Formula 3-7 which can then be reacted with sulfonyl chloride 3-8 to provide compounds of Formula Ic and Id.

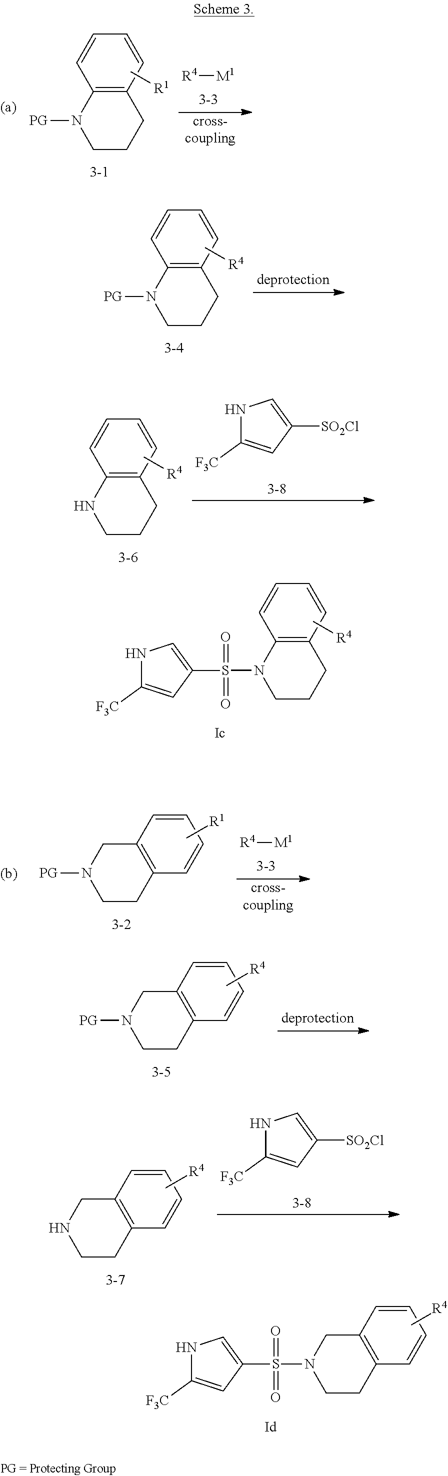

Compounds of Formula I, such as compounds of Formula Ie, can also be prepared as shown in Scheme 4. Protected cyclic amines 4-1 can be coupled with aryl or heteroaryl halides 4-2 (where R¹ can be Cl, Br, or I) under standard Buchwald conditions (e.g., in the presence of a palladium catalyst, such as tris(dibenzylideneacetone)dipalladium(0) [Pd₂(dba)₃], a ligand such as 2,2′-bis(diphenylphosphino)-1,1′-binaphthyl [BINAP] and abase [e.g., sodium tert-butoxide]) to afford compounds of Formula 4-3. Deprotection of the N-protecting group (e.g., Boc) can be achieved under suitable reaction conditions (e.g., with HCl for the removal of Boc) to provide amines of Formula 4-4 which can then be reacted with sulfonyl chloride 4-5 to provide compounds of Formula Ie.

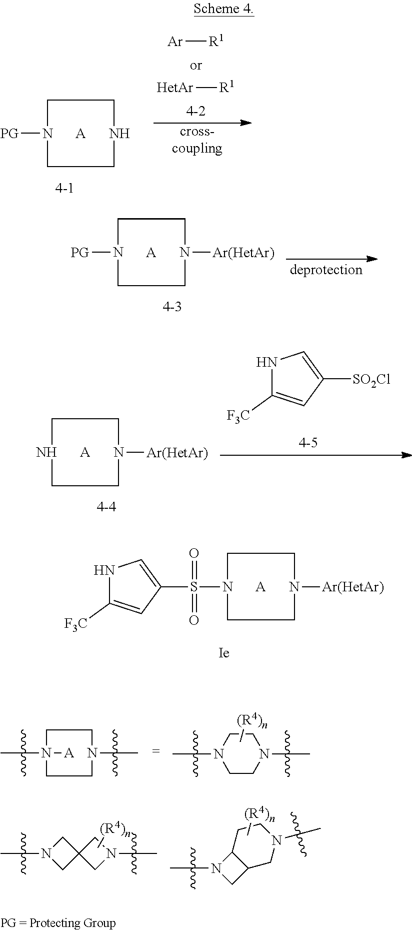

Compounds of Formula I, such as compounds of Formula If, Ig, Ih, and Ii, can also be prepared as shown in Scheme 5. Bis(2-chloroethyl)amine hydrochloride, 5-1, can be reacted with amines 5-2, 5-3 or 5-4 to afford piperazines of Formula 5-5, Formula 5-6 or Formula 5-7. Reaction with sulfonyl chloride 5-8 in the presence of a base such as triethylamine or N,N-diisopropylethylamine can afford compounds of Formula If, Ig, and Ih. Alternatively, alcohols of Formula 5-9 can be converted to the corresponding mesylates 5-10 by reaction with methanesulfonyl chloride. The mesylates can then be reacted with suitably protected piperazines of Formula 5-11 to afford substituted piperazines 5-12. Deprotection of the N-protecting group (e.g., Boc) can be achieved under suitable reaction conditions (e.g., with HCl for the removal of Boc) to provide the amines of Formula 5-13 which can then be reacted with sulfonyl chloride 5-8 to provide compounds of Formula II.

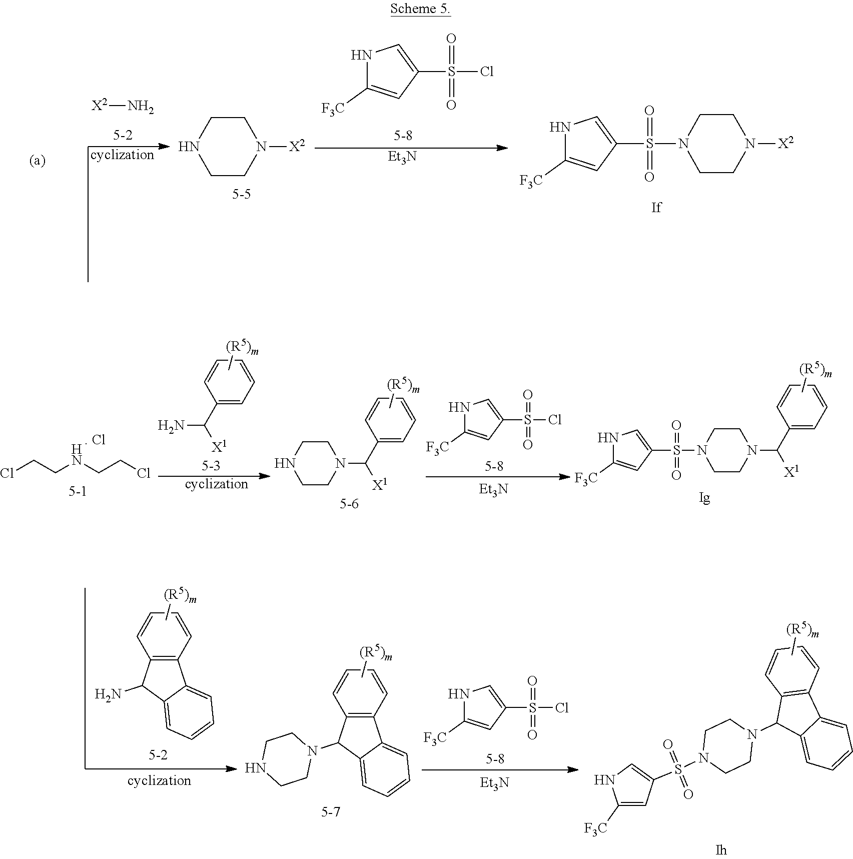

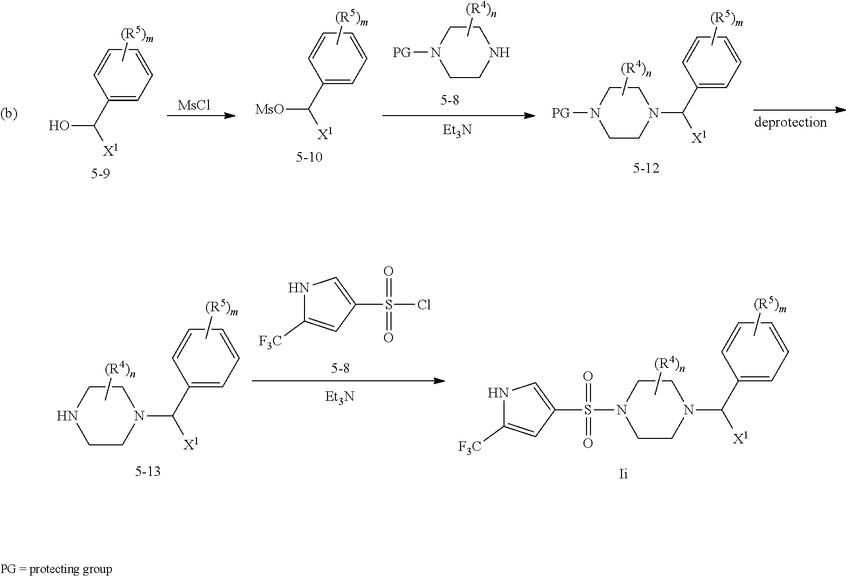

Compounds of Formula I, such as compounds of Formula Ij, Ik, Il, Im, In, Io, and Ip, can also be prepared as shown in Scheme 6. Suitable starting materials containing a nitrile as in 6-1, which can be synthesized through Scheme 5, can be reduced with lithium aluminum hydride to provide the corresponding aldehydes 6-2 or the corresponding amines 6-3. Aldehydes of Formula 6-2 can either be reduced to alcohols of Formula Ij upon treatment with a reducing agent such as sodium borohydride or reacted with amines of Formula 6-4 and sodium triacetoxyborohydride to afford compounds of Formula Ik. Compounds of Formula In, Io, and Ip can be obtained through the reaction of amines 6-3 with either carbamic chlorides of Formula 6-5, carboxylic acids of Formula 6-6 under standard amide formation conditions (e.g., HATU and triethylamine), or sulfonyl chlorides of Formula 6-7. Compounds of Formula II and Im can be obtained through the hydrolysis of nitriles 6-1 with an aqueous mixture of hydrogen peroxide and ammonia or through cycloaddition with sodium azide, respectively.

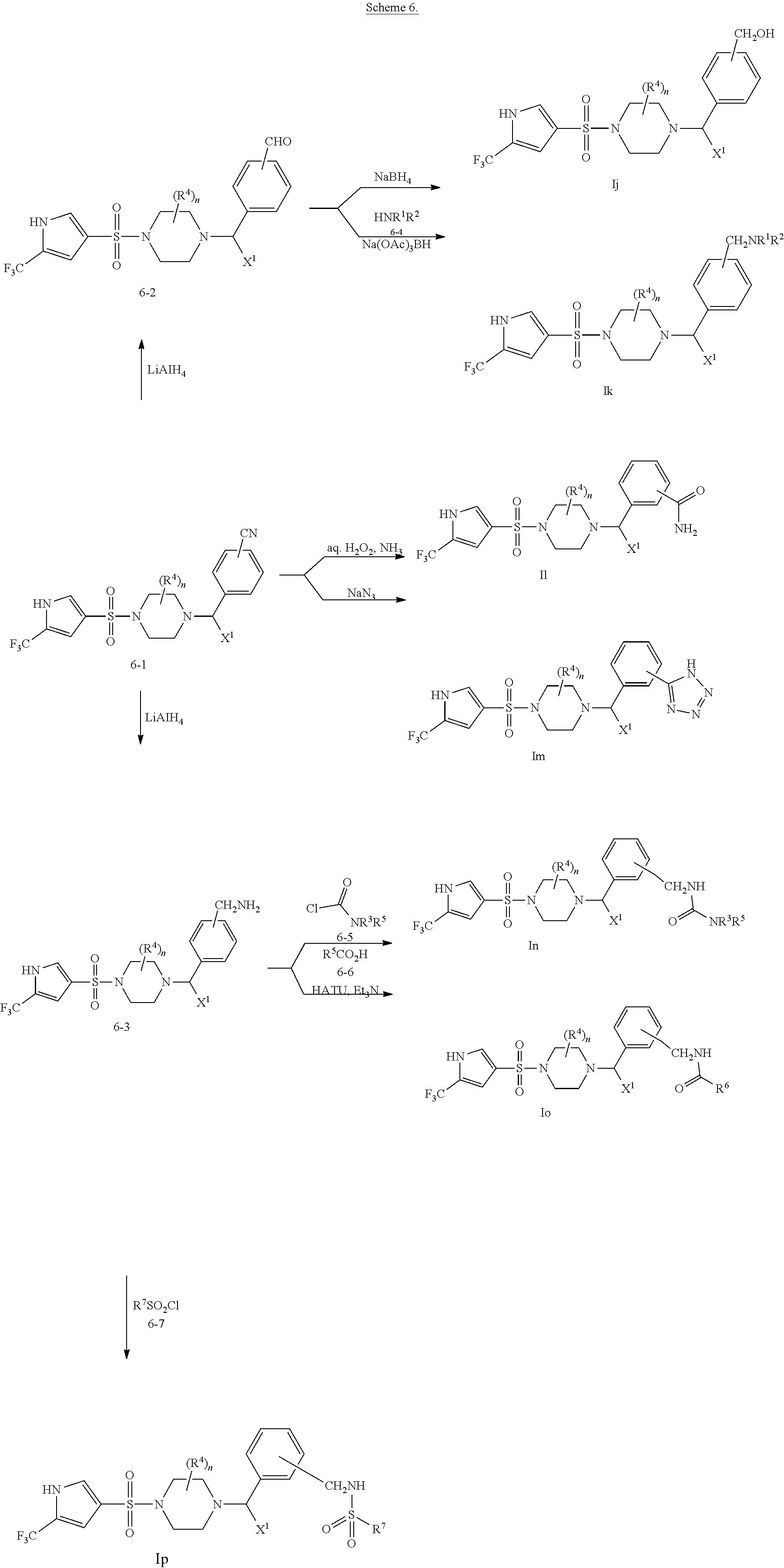

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of USP18. Compounds which inhibit USP18 are useful in providing a means of preventing the growth or inducing apoptosis in tumors and/or in providing a means of treating viral infections. One cause in the failure rate of immune modulating therapies is deficiencies in interferon-dependent signaling pathways. These IFN pathways stimulate the production of interferon stimulated genes that promote the innate immune system and apoptosis. USP18 is a de-ISGylating enzyme and negative regulator of IFN signaling. USP18 has been shown to have two important roles in maintaining this regulation, scaffolding and catalytic. The scaffolding role of USP18 is important to shut off continued signaling from exogenous IFN stimulation occurring as it shuttles in connection with STAT2 towards IFNAR2 to displace JAK1 phosphorylation at the internal membrane and preventing binding of the IFN-IFN-receptor axis. The enzymatic function of USP18 is to catalytically remove ISG15 chain from important protein conjugates such as ADAR, MX1, GPB1, and OASL. Due to certain IFN-rich TMEs, certain cancers have a greater dependency on USP18 to evade IFN-mediated cell death. It is therefore anticipated that the compounds of the disclosure are useful in treating or preventing proliferative disorders such as cancers. In particular, tumors with activating mutants of receptor tyrosine kinases or upregulation of receptor tyrosine kinases or with defects in DNA damage repair mechanisms may be particularly sensitive to the inhibitors of the present application. Additionally, USP18 is a negative regulator of IFN signaling and is associated with enhancing certain viral infections. Since loss of USP18 function enhances IFN-stimulated cellular antiviral responses, it is anticipated that compounds of the disclosure are useful in treating viral infections.

Compounds in this application may have selective activities towards USP18 and exhibit selectivity for USP18 over other related deubiquitinases, such as USP7. Selective activity for USP18 over other deubiquitinating enzymes can prevent off-target effects. These USP18 inhibitors alone or in combination with other therapeutic agent(s) can be used in treatment of cancer.

In some embodiments, the disclosure provides a method for treating a USP18-related disorder in a patient in need thereof, comprising the step of administering to said patient a compound of the disclosure, or a pharmaceutically acceptable composition thereof.

For example, the compounds of the disclosure may be useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

In some embodiments, the cancer is selected from bladder cancer, breast cancer, cervical cancer, colorectal cancer, cancer of the small intestine, colon cancer, rectal cancer, cancer of the anus, endometrial cancer, gastric cancer, head and neck cancer, kidney cancer, liver cancer, lung cancer, ovarian cancer, prostate cancer, testicular cancer, uterine cancer, vulvar cancer, esophageal cancer, gall bladder cancer, pancreatic cancer, stomach cancer, thyroid cancer, parathyroid cancer, neuroendocrine cancer, skin cancer, and brain cancer.

In some embodiments, the cancer is selected from bladder cancer, urothelial carcinoma, squamous cell carcinoma, adenocarcinoma, breast cancer, hormone R positive breast cancer, triple negative breast cancer, cervical cancer, colorectal cancer, cancer of the small intestine, colon cancer, rectal cancer, cancer of the anus, endometrial cancer, gastric cancer, gastrointestinal stromal tumors, head and neck cancer, cancers of the larynx, cancers of the hypopharynx, cancers of the nasopharynx, cancers of the oropharynx, cancers of the lips, cancers of the mouth, squamous head and neck cancer, kidney cancer, renal cell carcinoma, urothelial carcinoma, sarcoma, Wilms tumor, liver cancer, hepatocellular carcinoma, intrahepatic cholangiocellular carcinoma, hilar cholangiocellular carcinoma, perihilar cholangiocellular carcinoma, distal extrahepatic cholangiocellular carcinoma, liver angiosarcoma, hepatoblastoma, lung cancer, adenocarcinoma, small cell lung cancer, non-small cell lung carcinoma, parvicellular carcinoma, non-parvicellular carcinoma, bronchial carcinoma, bronchial adenoma, pleuropulmonary blastoma, ovarian cancer, prostate cancer, testicular cancer, uterine cancer, vulvar cancer, esophageal cancer, gall bladder cancer, pancreatic cancer, exocrine pancreatic carcinoma, stomach cancer, thyroid cancer, parathyroid cancer, neuroendocrine cancer, pheochromocytoma, Merkel cell cancer, neuroendocrine carcinoma, skin cancer, squamous cell carcinoma, Kaposi sarcoma, Merkel cell skin cancer, brain cancer, astrocytoma, medulloblastoma, ependymoma, neuro-ectodermal tumors, and pineal tumors.

In some embodiments, the cancer is selected from urothelial carcinoma, squamous cell carcinoma, adenocarcinoma, hormone R positive breast cancer, triple negative breast cancer, cervical cancer, colorectal cancer, cancer of the small intestine, colon cancer, rectal cancer, cancer of the anus, endometrial cancer, gastrointestinal stromal tumors, cancers of the larynx, cancers of the hypopharynx, cancers of the nasopharynx, cancers of the oropharynx, cancers of the lips, cancers of the mouth, squamous head and neck cancer, renal cell carcinoma, urothelial carcinoma, sarcoma, Wilms tumor, hepatocellular carcinoma, intrahepatic cholangiocellular carcinoma, hilar cholangiocellular carcinoma, perihilar cholangiocellular carcinoma, distal extrahepatic cholangiocellular carcinoma, liver angiosarcoma, hepatoblastoma, adenocarcinoma, small cell lung cancer, non-small cell lung carcinoma, parvicellular carcinoma, non-parvicellular carcinoma, bronchial carcinoma, bronchial adenoma, pleuropulmonary blastoma, ovarian cancer, prostate cancer, testicular cancer, uterine cancer, vulvar cancer, esophageal cancer, gall bladder cancer, exocrine pancreatic carcinoma, stomach cancer, thyroid cancer, parathyroid cancer, pheochromocytoma, Merkel cell cancer, neuroendocrine carcinoma, squamous cell carcinoma, Kaposi sarcoma, Merkel cell skin cancer, astrocytoma, medulloblastoma, ependymoma, neuro-ectodermal tumors, and pineal tumors.

In some embodiments, adrenal cancer, thyroid cancer, myeloid cancer, eye cancer, pancreatic cancer, prostate cancer, head and neck cancer, cancer of the uterus, ampullary cancer, cervical cancer, pleural cancer, bone cancer, lung cancer, breast cancer, ovarian cancer, fallopian tube cancer, cancer of the fibroblasts, liver cancer, bowel cancer, brain cancer, central nervous system cancer, soft tissue cancer, cancer of the biliary tract, esophageal cancer, stomach cancer, skin cancer, cancer of the peripheral nervous system, bladder cancer, cancer of the urinary tract, cancer of the lymphatic system, vulvar cancer, vaginal cancer, kidney cancer, and testicular cancer. In some embodiments, the pancreatic cancer is pancreatic ductal adenocarcinoma (PDAC). In some embodiments, the head and neck cancer is head and neck squamous cancer (HNSC). In some embodiments, the cancer is selected from pancreatic ductal adenocarcinoma (PDAC) and head and neck squamous cancer (HNSC).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglobulinemia, hairy cell lymphoma, chronic myelogenic lymphoma, acute lymphoblastic lymphoma, AIDS-related lymphomas, and Burkitt's lymphoma.

In some embodiments, provided herein is a method of treating cancer comprising administering to a patient in need thereof a therapeutically effect amount of a compound of the disclosure. In some embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

In some embodiments the cancer is selected from head and neck cancer, pancreatic cancer, cervical cancer, lung cancer, breast cancer, esophageal cancer, glioma, and glioblastoma.

The compounds of the disclosure can also be useful in the inhibition of tumor metastases.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

Compounds which inhibit USP18 are useful for treating viral infections, and viral diseases and conditions. Examples of viral infections include, but are not limited to, Lymphocytic choriomeningitis virus, human immunodeficiency virus (HIV), human papillomavirus (HPV), hepatitis B virus (HBV), hepatitis C virus (HCV), Epstein-Barr virus (EBV), cytomegalovirus (CMV), herpes simplex viruses (HSV), Varicella-Zoster virus (VZV), Zika virus, influenza virus, flavivirus, Enteroviruses, and Coronavirus

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a USP18 variant with a compound described herein includes the administration of a compound described herein to an individual or patient, such as a human, having a USP18 variant, as well as, for example, introducing a compound described herein into a sample containing a cellular or purified preparation containing the USP18 variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

In some embodiments, the USP18 inhibitor compound is administered in combination with an immunotherapy, a targeted therapy, a chemotherapy, and/or radiation therapy. In some embodiments, the active agents of the combination therapy have different and complementary mechanisms of action. In some embodiments, the active agents of the combination therapy have a synergistic and/or more durable therapeutic effect on a target disease, disorder, or condition. In some embodiments, the combination of active agents allows for a dose adjustment of one or more of the active agents, which can reduce adverse effects associated with one or more of the active agents.

In some embodiments, USP18 inhibitor compounds of the present disclosure are used in combination with one or more immune checkpoint inhibitors for the treatment of diseases, such as cancer. Examples of immune checkpoint inhibitors include inhibitors against immune checkpoint molecules such as CBL-B, CD20, CD28, CD40, CD122, CD96, CD73, CD47, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, IDO, KIR LAG3, TIM3, TIGIT, CD112R, VISTA, PD-1, PD-L1, and PD-L2. In some embodiments, the immune checkpoint inhibitor is a costimulatory molecule selected from CD27, CD28, CD40, ICOS, OX40, GITR, and CD137. In some embodiments, the USP18 inhibitor compounds are used in combination with one or more agents selected from KIR inhibitors, TIGIT inhibitors, LAIR1 inhibitors, CD160 inhibitors, 2B4 inhibitors, and TGFR beta inhibitors. In some embodiments, the inhibitor of an immune checkpoint molecule is an anti-PD1 antibody, anti-PD-L1 antibody, or anti-CTLA-4 antibody. In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, retifanlimab, SHR-1210, PDR001, MGA012, PDR001, AB122, or AMP-224. Other anti-cancer agent(s) include antibody therapeutics such as 4-1BB (e.g. urelumab, utomilumab).

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), durvalumab (Imfinzi®), atezolizumab (Tecentriq®), Avelumab (Bavencio®) or MSB0010718C.

In some embodiments, the inhibitor of an immune checkpoint molecule is an inhibitor of PD-1 and PD-L1, e.g., an anti-PD-1/PD-L1 bispecific antibody. In some embodiments, the anti-PD-1/PD-L1 is MCLA-136.

In some embodiments, the inhibitor of an immune checkpoint molecule is an inhibitor of CD137 and PD-L1, e.g., a CD137×PD-L1 bispecific antibody. In some embodiments, the CD137×PD-L1 bispecific antibody is the inhibitor MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the USP18 inhibitor compounds are used in combination with one or more bispecific antibodies. In some embodiments, one of the domains of the bispecific antibody targets PD-1, PD-L1, CTLA-4, GITR, OX40, TIM3, LAG3, CD137, ICOS, CD3, or TGFβ receptor.

In some embodiments, the USP18 inhibitor compounds can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds of the disclosure can be used in combination with one or more PD-L1 targeting small molecules. Examples of PD-L1 targeting small molecules include, but are not limited to, INCB086550, INCB099280 and INCB090244.

In some embodiments, the USP18 inhibitor compounds are used in combination with one or more agonist of immune checkpoint molecules. Examples of agonists of immune checkpoint molecules include OX40, CD27, GITR, and CD137 (also known as 4-1BB).

In some embodiments, the USP18 inhibitor compounds are used in combination with one or more inhibitors enhancing T cell receptor signaling such as inhibitors of diacylglycerol kinase (DGK) molecules, such as DGK-alpha, DGK-zeta and dual inhibitors of both DGK-alpha and DGK-zeta. Examples of DGK inhibitors include BAY 2965501, BMS-332, BMS-502, BMS-684, BMS-496, AMB639752, and JNJ-3790339.

In some embodiments, USP18 inhibitor compounds of the present disclosure are used in combination with one or more immune activators for the treatment of diseases, such as cancer. Examples of immune activators include molecules that activate interferon signaling, including recombinant forms of interferon-alpha, (IFNα), recombinant forms of interferon beta (IFN-β), STING agonists, Toll-like receptor agonists, Nod-Like-receptor (NLR) agonists, PARP7 inhibitors, RIG-1 agonists, MDA5 agonists, MAVS agonists, TREX1 inhibitors, DDX41 agonists, DAI agonists, IFI16 agonists, LRRFIP1 agonists, DHX9 agonists, DHX36 agonists, KU70 agonists.

In some embodiments, the immune activator molecule is an agonist of interferon receptor. In some embodiments, the interferon receptor agonist is a recombinant form of interferon alpha, such as Referon-A, Alferon N, Fiblaferon, Wellferon, Roferon, Intron A, Berofor, Pegasys, and ropeginterferon alfa-2b (Besremi). In some embodiments, the interferon receptor agonist is a recombinant form of interferon beta, such as Avonex, Rebif, Betaferon, Betaseron, Extavia, and Pegridy.

In some embodiments, the immune activator molecule is an agonist of Toll-like receptors (TLR). Some examples of agonists of TLRs are, but are not limited to, Poly-ICLC, Hespecta, Mobilan, Entolimod, Imiquimod, Resiquimod, Lefitolimod, Vesatolimod, Rintatolimod, Agatolimod, SMP-105, IHP-3102, IMO-2055, NGN-1706, ANA773, OM-174, ISS1018, Eritran, VTX-2337, SD-101, 852A, and PUL-042.

In some embodiments, the USP18 inhibitor compounds of the present disclosure can be used in combination with other methods of treating cancers. Examples of other methods of treating cancer include chemotherapy, radiation therapy, tumor-targeted therapy, adjuvant therapy, immunotherapy, and surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, STING agonists, RIG-I agonists, oncolytic virotherapy and immunomodulating small molecules, including thalidomide or JAK1/2 inhibitor, PI3Kδ inhibitor, and the like. In some embodiments, the USP18 inhibitor compounds are administered in combination with one or more anti-cancer drugs, such as a chemotherapeutic agents and agents that induce DNA damage. Examples of chemotherapeutic agents include: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Examples of other anti-cancer agents include antibody therapeutics such as trastuzumab (Herceptin), antibodies to costimulatory molecules such as CTLA-4 (e.g., ipilimumab), 4-1BB (e.g. urelumab, utomilumab), antibodies to PD-1 and PD-L1, or antibodies to cytokines (IL-10, TGF-β, etc.). Examples of antibodies to PD-1 and/or PD-L1 that can be combined with the USP18 inhibitor compounds of the present disclosure for the treatment of cancer or infections (such as viral, bacteria, fungus, and parasite infections) include nivolumab, pembrolizumab, atezolizumab, durvalumab, avelumab, and SHR-1210.

In some embodiments, the USP18 inhibitor compounds of the present disclosure can be used in combination with other methods of treating viral infections and viral diseases. Examples of treatments for viral disease include molecules that activate interferon signaling, including recombinant forms of interferon-alpha, (IFNα), recombinant forms of interferon beta (IFN-β), STING agonists, Toll-like receptor agonists, Nod-Like-receptor (NLR) agonists, PARP7 inhibitors, RIG-1 agonists, MDA5 agonists, MAVS agonists, TREX1 inhibitors, DDX41 agonists, DAI agonists, IFI16 agonists, LRRFIP1 agonists, DHX9 agonists, DHX36 agonists, KU70 agonists.

In some embodiments, the interferon receptor agonist is a recombinant form of interferon alpha, such as Referon-A, Alferon N, Fiblaferon, Wellferon, Roferon, Intron A, Berofor, Pegasys, and ropeginterferon alfa-2b (Besremi). In some embodiments, the interferon receptor agonist is a recombinant form of interferon beta, such as Avonex, Rebif, Betaferon, Betaseron, Extavia, and Pegridy.

In some embodiments examples of treatments for viral disease include molecules that activate interferon signaling, including STING agonists. Examples of STING agonists include ADU-S100, MK-1454, MK-2118, SB11285, GSK3745417, BMS-986301, E7766, TAK-676, SNX281, SYNB1891, and VB-85247.

In some embodiments, the USP18 inhibitor compounds of the present disclosure can be used in combination with other methods of treating viral infections and viral diseases. In some embodiments, the USP18 inhibitor compounds of the present disclosure can be used in combination with other methods of treating viral infections, such HIV. Examples of treatments for HIV include Ziagen, Emtriva, Epivir, Viread, Retrovir, Pifeltro, Sustiva, Intelence, Viramune, Viramune XR, Edurant, Reyataz, Prezista, Lexiva, Norvir, Aptivus, Fuzeon, Selzentry, Vocabria, Isentress, Isentress HD, Rukobia, Trogarzo, Sunlenca, Tybost, Epzicom, Triumeq, Triumeq PD, Trizivir, Evotaz, Biktarvy, Cabenuva, Prezcobix, Sumtuza, and Dovato, Juluca, Delstrigo, Atripla, Symfi, Genvoya, Stribild, Odefsey, Complera, Descovy, Truvada, Cimduo, Combivir, and Kaletra.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal, and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating USP18 in tissue samples, including human, and for identifying USP18 inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes USP18 assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²¹I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CDH₂(i.e., deuteromethyl), —CD₂H (i.e., dideuteromethyl), or —CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CDH₂, —CD₂H, or —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₆ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₆ alkyl-, alkylene, alkenylene and alkynylene linking groups, as described herein, are optionally replaced by deuterium atoms.

In some embodiments, the compound provided herein (e.g., the compound of any of Formulas I-XIV), or a pharmaceutically acceptable salt thereof, comprises at least one deuterium atom.

In some embodiments, the compound provided herein (e.g., the compound of any of Formulas I-XIV), or a pharmaceutically acceptable salt thereof, comprises two or more deuterium atoms.

In some embodiments, the compound provided herein (e.g., the compound of any of Formulas I-XIV), or a pharmaceutically acceptable salt thereof, comprises three or more deuterium atoms.

In some embodiments, for a compound provided herein (e.g., the compound of any of Formulas I-XIV), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro USP18 labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind USP18 by monitoring its concentration variation when contacting with USP18, through tracking of the labeling. For example, a test compound (labeled) can be evaluated for its ability to reduce binding of another compound which is known to bind to USP18 (i.e., standard compound). Accordingly, the ability of a test compound to compete with the standard compound for binding to USP18 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of USP18-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)).

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

### Example 1. 1-(5-Chloro-2-methylphenyl)-4-((5-(trifluoromethyl)-1H-pyrrol-3-yl)sulfonyl)piperazine

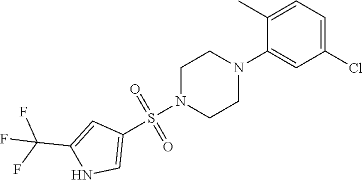 [SMILES: Cc1ccc(Cl)cc1N1CCN(S(=O)(=O)c2c[nH]c(C(F)(F)F)c2)CC1]

To a solution of 5-(trifluoromethyl)-1H-pyrrole-3-sulfonyl chloride (152.2 mg, 0.652 mmol) in dichloromethane (6 mL) at room temperature was added 1-(5-chloro-2-methylphenyl)piperazine (138 μL, 0.782 mmol) and triethylamine (272 μL, 1.955 mmol). The mixture was allowed to stir at room temperature for 8 hours and then concentrated in vacuo. The crude sample was taken up in acetonitrile and purified directly by preparative reverse-phase HPLC (pH=2) to give 60 mg of desired product (0.147 mmol, 22.6%). LC-MS calculated for C₁₆H₁₈ClF₃N₃O₂S (M+H)⁺: m/z=408.1. found 408.1. ¹H NMR (400 MHz, DMSO-d₆) δ 13.22 (s, 1H), 7.70 (d, J=1.7 Hz, 1H), 7.17 (d, J=7.9 Hz, 1H), 7.07-6.99 (m, 2H), 6.96 (t, J=1.4 Hz, 1H), 3.01 (s, 4H), 2.95 (t, J=4.7 Hz, 4H), 2.14 (s, 3H).

### Examples 2-41

Examples 2-41 in Table 1 were prepared according to the procedures described in Example 1, using appropriately substituted starting materials.

TABLE 1

| Ex. | | | LCMS | |
|---|---|---|---|---|
| No. | Name | Structure | [M + H]⁺ | ¹H NMR |
| 2 | 1-(3-Nitrophenyl)-4- ((5-(trifluoromethyl)- 1H-pyrrol-3- yl)sulfonyl)piperazine | 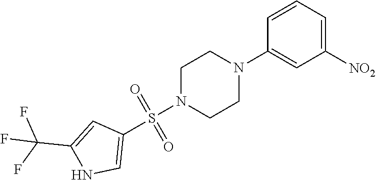 [SMILES: O=[N+]([O-])c1cccc(N2CCN(S(=O)(=O)c3c[nH]c(C(F)(F)F)c3)CC2)c1] | 405.1 | ¹H NMR (400 MHz, DMSO- d₆) δ 13.23 (s, 1H), 7.74-7.58 (m, 3H), 7.48 (t, J = 8.1 Hz, 1H), 7.40 (dd, J = 8.3, 2.5 Hz, 1H), 6.95 (s, 1H), 3.38 (t, J = 5.1 Hz, 4H), 3.01 |
| | | | | (dd, J = 6.2, 3.9 |
| | | | | Hz, 4H). |
| 3 | 1-(2-Chloro-6- methylphenyl)-4-((5- (trifluoromethyl)-1H- pyrrol-3- yl)sulfonyl)piperazine | 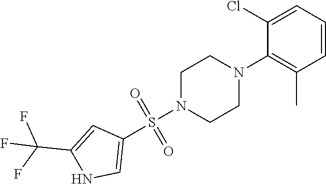 [SMILES: Cc1cccc(Cl)c1N1CCN(S(=O)(=O)c2c[nH]c(C(F)(F)F)c2)CC1] | 408.0 | ¹H NMR (400 MHz, DMSO- d₆) δ 13.19 (s, 1H), 7.70 (t, J = 2.6 Hz, 1H), 7.22 (d, J = 7.8 Hz, 1H), 7.16 (d, J = 7.4 Hz, 1H), 7.07 (t, J = 7.7 Hz, 1H), 6.96 (s, 1H), |
| | | | | 3.36 (m, partial |
| | | | | overlap with |
| | | | | water peak), |
| | | | | 3.27 (m, partial |
| | | | | overlap with |
| | | | | water peak), |
| | | | | 2.96 (d, J = 11.5 |
| | | | | Hz, 2H), 2.75 (t, |
| | | | | J = 9.8 Hz, 2H), |
| | | | | 2.23 (s, 3H).^{†} |
| 4 | 1-(2,4- Dichlorophenyl)-4- ((5-(trifluoromethyl)- 1H-pyrrol-3- yl)sulfonyl)piperazine | 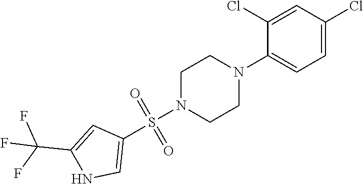 [SMILES: O=S(=O)(c1c[nH]c(C(F)(F)F)c1)N1CCN(c2ccc(Cl)cc2Cl)CC1] | 428.1 | ¹H NMR (400 MHz, DMSO- d₆) δ 13.24 (s, 1H), 7.71 (dd, J = 3.2, 1.7 Hz, 1H), 7.55 (d, J = 2.5 Hz, 1H), 7.39 (dd, J = 8.7, 2.5 Hz, 1H), 7.20 (d, J = 8.7 Hz, 1H), 6.97 (s, |
| | | | | 1H), 3.04 (dd, J = |
| | | | | 16.1, 5.2 Hz, |
| | | | | 8H). |
| 5 | 1-(2,6- Dichlorophenyl)-4- ((5-(trifluoromethyl)- 1H-pyrrol-3- yl)sulfonyl)piperazine | 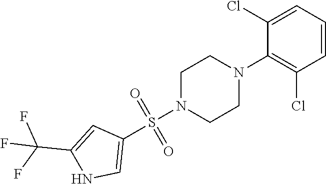 [SMILES: O=S(=O)(c1c[nH]c(C(F)(F)F)c1)N1CCN(c2c(Cl)cccc2Cl)CC1] | 428.0 | ¹H NMR (400 MHz, Methanol- d₄) δ 7.54-7.49 (d, J = 1.7 Hz, 1H), 7.38-7.31 (d, J = 8.1 Hz, 2H), 7.15-7.07 (t, J = 8.0 Hz, 1H), 6.92-6.87 (s, 1H), 3.30 (m, partial overlap |
| | | | | with solvent |
| | | | | peak), 3.19- |
| | | | | 3.12 (t, J = 4.9 |
| | | | | Hz, 4H)^{†} |
| 6 | 1-(4-Chloro-2- methylphenyl)-4-((5- (trifluoromethyl)-1H- pyrrol-3- yl)sulfonyl)piperazine | 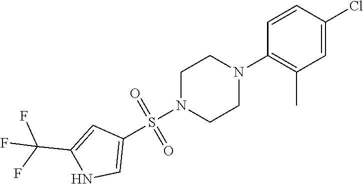 [SMILES: Cc1cc(Cl)ccc1N1CCN(S(=O)(=O)c2c[nH]c(C(F)(F)F)c2)CC1] | 408.0 | ¹H NMR (400 MHz, DMSO- d₆) δ 13.22 (s, 1H), 7.70 (dd, J = 3.3, 1.7 Hz, 1H), 7.25-7.16 (m, 2H), 7.06 (d, J = 8.5 Hz, 1H), 6.96 (s, 1H), 3.01 (t, J = 4.6 Hz, 4H), 2.91 (t, |
| | | | | J = 4.7 Hz, 4H), |
| | | | | 2.16 (s, 3H). |
| 7 | 1-Phenyl-4-((5- (trifluoromethyl)-1H- pyrrol-3- yl)sulfonyl)piperazine | 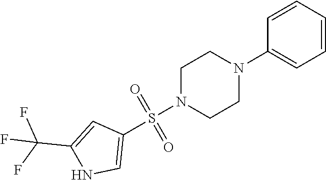 [SMILES: O=S(=O)(c1c[nH]c(C(F)(F)F)c1)N1CCN(c2ccccc2)CC1] | 360.0 | ¹H NMR (400 MHz, DMSO- d₆) δ 13.22 (s, 1H), 7.70 (t, J = 2.4 Hz, 1H), 7.22 (t, J = 7.8 Hz, 2H), 6.94 (d, J = 8.3 Hz, 3H), 6.82 (t, J = 7.3 Hz, 1H), 3.22 (t, J = 4.9 |
| | | | | Hz, 4H), 2.98 (t, |
| | | | | J = 5.0 Hz, 4H). |
| 8 | 1-(2-Ethylphenyl)-4- ((5-(trifluoromethyl)- 1H-pyrrol-3- yl)sulfonyl)piperazine | 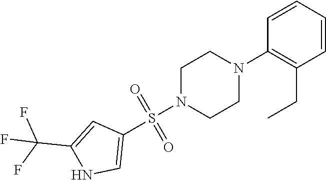 [SMILES: CCc1ccccc1N1CCN(S(=O)(=O)c2c[nH]c(C(F)(F)F)c2)CC1] | 388.2 | ¹H NMR (400 MHz, Methanol- d₄) δ 7.55-7.50 (d, J = 1.7 Hz, 1H), 7.25-7.19 (d, J = 7.4 Hz, 1H), 7.21-7.11 (dd, J = 6.3, 1.8 Hz, 2H), 7.11- 7.02 (td, J = 6.8, 2.4 Hz, 1H), |
| | | | | 6.93-6.88 (s, |
| | | | | 1H), 3.18-3.13 |
| | | | | (s, 4H), 3.04- |
| | | | | 2.97 (t, J = 4.8 |
| | | | | Hz, 4H), 2.70- |
| | | | | 2.60 (q, J = 7.5 |
| | | | | Hz, 2H), 1.22- |
| | | | | 1.13 (t, J = 7.5 |
| | | | | Hz, 3H). |
| 9 | 1-(4-Fluoro-2- methylphenyl)-4-((5- (trifluoromethyl)-1H- pyrrol-3- yl)sulfonyl)piperazine | 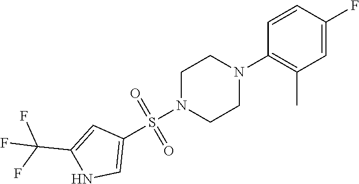 [SMILES: Cc1cc(F)ccc1N1CCN(S(=O)(=O)c2c[nH]c(C(F)(F)F)c2)CC1] | 392.1 | ¹H NMR (400 MHz, DMSO- d₆) δ 13.22 (s, 1H), 7.70 (dd, J = 3.2, 1.7 Hz, 1H), 7.13-6.91 (m, 4H), 3.01 (t, J = 4.7 Hz, 4H), 2.89 (t, J = 4.8 Hz, 4H), 2.17 (s, 3H). |
| 10 | 1-((5- (Trifluoromethyl)-1H- pyrrol-3-yl)sulfonyl)- 4-(2- (trifluoromethyl) phenyl)piperazine | 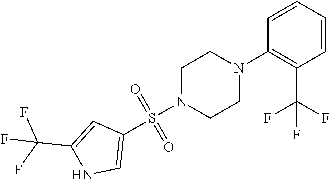 [SMILES: O=S(=O)(c1c[nH]c(C(F)(F)F)c1)N1CCN(c2ccccc2C(F)(F)F)CC1] | 428.2 | ¹H NMR (400 MHz, Methanol- d₄) δ 7.68-7.58 (dd, J = 14.1, 7.6 Hz, 2H), 7.56-7.50 (m, 2H), 7.37-7.29 (t, J = 7.6 Hz, 1H), 6.92-6.87 (s, 1H), 3.17- 3.10 (d, J = 4.8 |
| | | | | Hz, 4H), 3.07- |
| | | | | 3.00 (t, J = 4.7 |
| | | | | Hz, 4H). |
| 11 | 1-(3-Chloro-2- methylphenyl)-4-((5- (trifluoromethyl)-1H- pyrrol-3- yl)sulfonyl)piperazine | 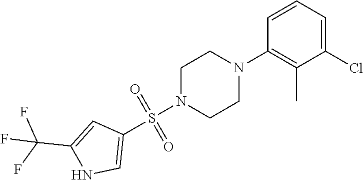 [SMILES: Cc1c(Cl)cccc1N1CCN(S(=O)(=O)c2c[nH]c(C(F)(F)F)c2)CC1] | 408.0 | ¹H NMR (400 MHz, DMSO- d₆) δ 13.22 (s, 1H), 7.70 (dd, J = 3.2, 1.7 Hz, 1H), 7.23-7.12 (m, 2H), 7.06 (dd, J = 7.4, 2.0 Hz, 1H), 6.96 (s, 1H), 3.03 (br.s, 4H), 2.94 (t, J = |
| | | | | 4.7 Hz, 4H), |
| | | | | 2.21 (s, 3H). |
| 12 | 1-(3-Methyl-[1,1′- biphenyl]-4-yl)-4-((5- (trifluoromethyl)-1H- pyrrol-3- yl)sulfonyl)piperazine | 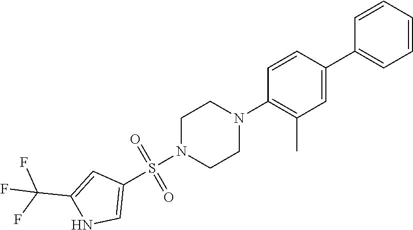 [SMILES: Cc1cc(-c2ccccc2)ccc1N1CCN(S(=O)(=O)c2c[nH]c(C(F)(F)F)c2)CC1] | 450.2 | ¹H NMR (400 MHz, DMSO- d₆) δ 13.25- 13.21 (s, 1H), 7.74-7.68 (dd, J = 3.2, 1.7 Hz, 1H), 7.64-7.57 (m, 2H), 7.49- 7.39 (m, 4H), 7.36-7.27 (t, J = 7.3 Hz, 1H), 7.16-7.10 (d, J = 8.0 Hz, 1H), 7.00-6.95 (s, |
| | | | | 1H), 3.07-3.01 |
| | | | | (d, J = 5.6 Hz, |
| | | | | 4H), 3.01-2.94 |
| | | | | (t, J = 4.6 Hz, |
| | | | | 4H), 2.27-2.22 |
| | | | | (s, 3H). |
| 13 | 1-([1,1′-Biphenyl]-2- yl)-4-((5- (trifluoromethyl)-1H- pyrrol-3- yl)sulfonyl)piperazine | 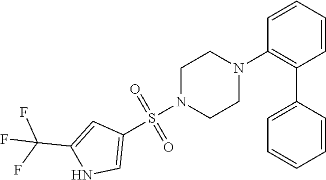 [SMILES: O=S(=O)(c1c[nH]c(C(F)(F)F)c1)N1CCN(c2ccccc2-c2ccccc2)CC1] | 436.2 | ¹H NMR (400 MHz, DMSO- d₆) δ 13.24- 13.20 (s, 1H), 7.68-7.62 (dd, J = 3.2, 1.7 Hz, 1H), 7.53-7.44 (m, 2H), 7.34- 7.23 (m, 4H), 7.23-7.16 (dd, J = 7.8, 1.8 Hz, |
| | | | | 1H), 7.15-7.06 |
| | | | | (t, J = 7.5 Hz, |
| | | | | 2H), 6.89-6.84 |
| | | | | (s, 1H), 2.87- |
| | | | | 2.80 (t, J = 4.7 |
| | | | | Hz, 4H), 2.77- |
| | | | | 2.70 (t, J = 4.7 |
| | | | | Hz, 4H). |
| 14 | 2-(4-((5- (Trifluoromethyl)-1H- pyrrol-3- yl)sulfonyl)piperazin- 1-yl)benzonitrile | 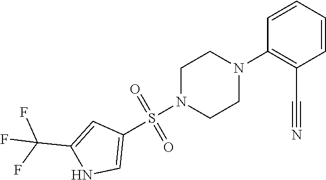 [SMILES: N#Cc1ccccc1N1CCN(S(=O)(=O)c2c[nH]c(C(F)(F)F)c2)CC1] | 385.2 | ¹H NMR (400 MHz, CDCl₃) δ 9.53-9.48 (br.s, 1H), 7.64- 7.59 (dd, J = 7.7, 1.6 Hz, 1H), 7.58-7.52 (td, J = 7.9, 1.6 Hz, 1H), 7.42-7.36 (dd, J = 3.1, 1.7 Hz, 1H), 7.15- |
| | | | | 7.04 (m, 2H), |
| | | | | 6.91-6.85 (dt, |
| | | | | J = 2.9, 1.5 Hz, |
| | | | | 1H), 3.36-3.24 |
| | | | | (m, 8H). |
| 15 | 1-(2-Methoxyphenyl)- 4-((5- (trifluoromethyl)-1H- pyrrol-3- yl)sulfonyl)piperazine | 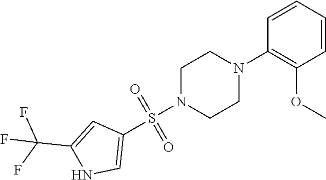 [SMILES: COc1ccccc1N1CCN(S(=O)(=O)c2c[nH]c(C(F)(F)F)c2)CC1] | 390.2 | ¹H NMR (400 MHz, Methanol- d₄) δ 7.56 (d, J = 1.7 Hz, 1H), 7.25-7.16 (m, 2H), 7.10-7.04 (m, 1H), 7.04- 6.95 (m, 1H), 6.93 (s, 1H), 3.89 (s, 3H), 3.36 (dd, J = |
| | | | | 6.6, 3.4 Hz, 4H), |
| | | | | 3.25 (t, J = 4.9 |
| | | | | Hz, 4H). |
| 16 | 2-(4-((5- (Trifluoromethyl)-1H- pyrrol-3- yl)sulfonyl)piperazin- 1-yl)aniline | 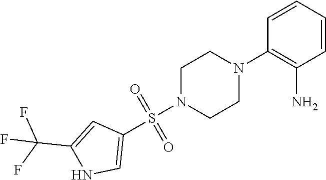 [SMILES: Nc1ccccc1N1CCN(S(=O)(=O)c2c[nH]c(C(F)(F)F)c2)CC1] | 375.1 | ¹H NMR (400 MHz, DMSO- d₆) δ 13.24 (s, 1H), 7.71 (dd, J = 3.2, 1.7 Hz, 1H), 7.19-7.12 (m, 1H), 7.07- 6.90 (m, 4H), 3.07 (br.s, 4H), 2.91 (t, J = 4.9 Hz, 4H). |
| 17 | 1-(Naphthalen-1-yl)- 4-((5- (trifluoromethyl)-1H- pyrrol-3- yl)sulfonyl)piperazine | 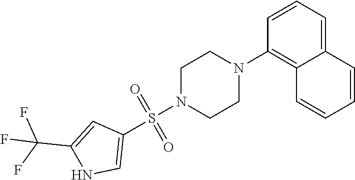 [SMILES: O=S(=O)(c1c[nH]c(C(F)(F)F)c1)N1CCN(c2cccc3ccccc23)CC1] | 410.1 | ¹H NMR (400 MHz, DMSO- d₆) δ 13.59 (s, 1H), 8.02 (m, 1H), 7.93-7.85 (m, 1H), 7.63 (d, J = 8.2 Hz, 1H), 7.50 (dt, J = 6.4, 3.4 Hz, 2H), 7.44 (t, J = 7.8 Hz, 1H), 7.15 |
| | | | | (d, J = 7.4 Hz, |
| | | | | 1H), 6.85 (dt, J = |
| | | | | 12.4, 3.5 Hz, |
| | | | | 2H), 3.32 (d, J = |
| | | | | 2.0 Hz, 4H), |
| | | | | 2.51 (m, 4H). |
| 18 | Methyl 4-methyl-3-(4- ((5-(trifluoromethyl)- 1H-pyrrol-3- yl)sulfonyl)piperazin- 1-yl)benzoate | 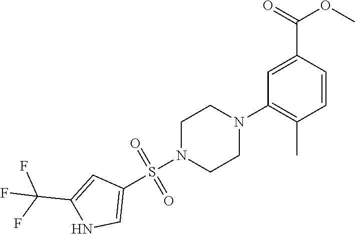 [SMILES: COC(=O)c1ccc(C)c(N2CCN(S(=O)(=O)c3c[nH]c(C(F)(F)F)c3)CC2)c1] | 432.1 | |
| 19 | 1-(3,5- Dichloropyridin-2-yl)- 4-((5- (trifluoromethyl)-1H- pyrrol-3- yl)sulfonyl)piperazine | 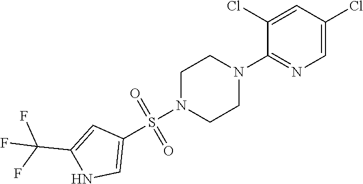 [SMILES: O=S(=O)(c1c[nH]c(C(F)(F)F)c1)N1CCN(c2ncc(Cl)cc2Cl)CC1] | 429.0 | ¹H NMR (400 MHz, DMSO- d₆) δ 13.25- 13.20 (s, 1H), 8.31-8.25 (d, J = 2.3 Hz, 1H), 8.09-8.04 (d, J = 2.3 Hz, 1H), 7.72-7.66 (dd, J = 3.1, 1.7 Hz, 1H), 6.97-6.93 |
| | | | | (s, 1H), 3.39- |
| | | | | 3.32 (m, partial |
| | | | | overlap with |
| | | | | water peak), |
| | | | | 3.05-2.98 (t, J = |
| | | | | 4.9 Hz, 4H).^{†} |
| 20 | 1-(3,5- Dichloropyridin-4-yl)- 4-((5- (trifluoromethyl)-1H- pyrrol-3- yl)sulfonyl)piperazine | 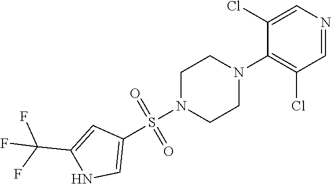 [SMILES: O=S(=O)(c1c[nH]c(C(F)(F)F)c1)N1CCN(c2c(Cl)cncc2Cl)CC1] | 429.0 | ¹H NMR (400 MHz, DMSO- d₆) δ 13.25- 13.21 (br.s, 1H), 8.49-8.44 (s, 2H), 7.74-7.68 (dd, J = 3.2, 1.7 Hz, 1H), 6.99- 6.95 (s, 1H), 3.40-3.34 (t, J = 4.8 Hz, 4H), |
| | | | | 3.07-3.00 (t, J = |
| | | | | 4.7 Hz, 4H). |
| 21 | 1-(3-Chloro-5- (trifluoromethyl) pyridin-2-yl)-4-((5- (trifluoromethyl)-1H- pyrrol-3- yl)sulfonyl)piperazine | 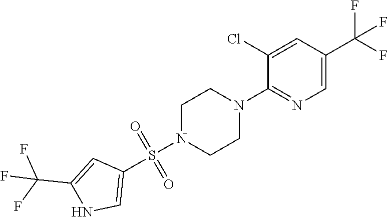 [SMILES: O=S(=O)(c1c[nH]c(C(F)(F)F)c1)N1CCN(c2ncc(C(F)(F)F)cc2Cl)CC1] | 463.1 | ¹H NMR (400 MHz, DMSO- d₆) δ 13.24- 13.19 (br.s, 1H), 8.58-8.53 (d, J = 2.1 Hz, 1H), 8.24-8.19 (d, J = 2.2 Hz, 1H), 7.72-7.67 (dd, J = 3.3, 1.7 Hz, 1H), 6.97-6.93 (br.s, 1H), 3.59- 3.52 (t, J = 4.9 |
| | | | | Hz, 4H), 3.07- |
| | | | | 3.00 (t, J = 4.9 |
| | | | | Hz, 4H). |
| 22 | 6-Bromo-1-((5- (trifluoromethyl)-1H- pyrrol-3-yl)sulfonyl)- 1,2,3,4- tetrahydroquinoline | 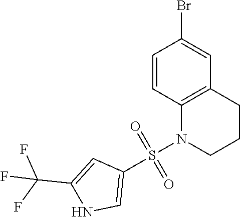 [SMILES: O=S(=O)(c1c[nH]c(C(F)(F)F)c1)N1CCCc2cc(Br)ccc21] | 409.0, 411.0 | ¹H NMR (400 MHz, DMSO- d₆) δ 13.10 (s, 1H), 7.63-7.55 (m, 2H), 7.39- 7.29 (m, 2H), 6.77 (s, 1H), 3.77-3.70 (m, 2H), 2.55 (m, partial overlap with solvent peak), 1.69 (qd, J = 6.5, 4.0 Hz, |
| | | | | 2H).^{†} |
| 23 | 7-Bromo-1-((5- (trifluoromethyl)-1H- pyrrol-3-yl)sulfonyl)- 1,2,3,4- tetrahydroquinoline | 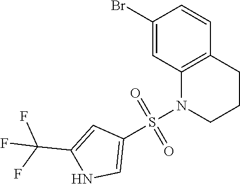 [SMILES: O=S(=O)(c1c[nH]c(C(F)(F)F)c1)N1CCCc2ccc(Br)cc21] | 409.1, 411.0 | ¹H NMR (400 MHz, DMSO- d₆) δ 13.14 (s, 1H), 7.77 (d, J = 2.0 Hz, 1H), 7.63 (d, J = 1.8 Hz, 1H), 7.25 (dd, J = 8.2, 2.0 Hz, 1H), 7.07 (d, J = 8.2 Hz, 1H), 6.75 (s, |
| | | | | 1H), 3.78-3.70 |
| | | | | (m, 2H), 1.75- |
| | | | | 1.64 (m, 2H).^{†} |
| 24 | 5-Bromo-1-((5- (trifluoromethyl)-1H- pyrrol-3-yl)sulfonyl)- 1,2,3,4- tetrahydroquinoline | 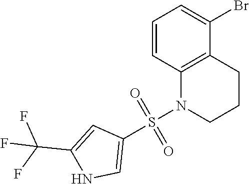 [SMILES: O=S(=O)(c1c[nH]c(C(F)(F)F)c1)N1CCCc2c(Br)cccc21] | 408.9, 410.9 | ¹H NMR (400 MHz, DMSO- d₆) δ 13.12 (s, 1H), 7.69-7.60 (m, 2H), 7.42 (d, J = 7.9 Hz, 1H), 7.15 (t, J = 8.2 Hz, 1H), 6.74 (s, 1H), 3.77-3.70 (m, 2H), 2.59- 2.51 (m, 2H), |
| | | | | 1.80-1.69 (m, |
| | | | | 2H). |
| 25 | 1-((5- (Trifluoromethyl)-1H- pyrrol-3-yl)sulfonyl)- 1,2,3,4- tetrahydroquinoline | 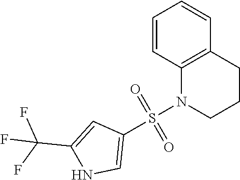 [SMILES: O=S(=O)(c1c[nH]c(C(F)(F)F)c1)N1CCCc2ccccc21] | 331.1 | ¹H NMR (400 MHz, DMSO- d₆) δ 13.05 (s, 1H), 7.66-7.59 (m, 1H), 7.55 (d, J = 1.7 Hz, 1H), 7.17 (td, J = 8.3, 7.6, 2.1 Hz, 1H), 7.13-7.01 (m, 2H), 6.65 (s, 1H), 3.78-3.71 |
| | | | | (m, 2H), 1.77- |
| | | | | 1.66 (m, 2H).^{†} |
| 26 | 7-Phenyl-2-((5- (trifluoromethyl)-1H- pyrrol-3-yl)sulfonyl)- 1,2,3,4- tetrahydroisoquinoline | 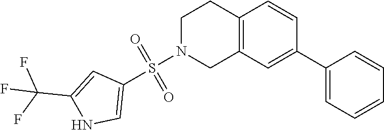 [SMILES: O=S(=O)(c1c[nH]c(C(F)(F)F)c1)N1CCc2ccc(-c3ccccc3)cc2C1] | 407.1 | ¹H NMR (400 MHz, DMSO- d₆) δ 13.15 (s, 1H), 7.72 (dd, J = 3.2, 1.7 Hz, 1H), 7.67-7.60 (m, 2H), 7.45 (ddt, J = 9.9, |
| | | | | 8.5, 2.1 Hz, 4H), |
| | | | | 7.39-7.31 (m, |
| | | | | 1H), 7.23 (d, J = |
| | | | | 7.8 Hz, 1H), |
| | | | | 6.98 (m, 1H), |
| | | | | 4.24 (s, 2H), |
| | | | | 3.27 (t, J = 5.9 |
| | | | | Hz, 2H), 2.92 (t, |
| | | | | J = 6.0 Hz, 2H). |
| 27 | 5-Fluoro-2-((5- (trifluoromethyl)-1H- pyrrol-3- yl)sulfonyl)isoindoline | 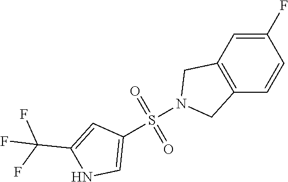 [SMILES: O=S(=O)(c1c[nH]c(C(F)(F)F)c1)N1Cc2ccc(F)cc2C1] | 335.0 | ¹H NMR (400 MHz, DMSO- d₆) δ 13.14- 13.10 (s, 1H), 7.76-7.71 (dd, J = 3.2, 1.7 Hz, 1H), 7.32-7.24 (dd, J = 8.4, 5.1 Hz, 1H), 7.15- 7.03 (ddd, J = 16.9, 8.3, 2.5 |
| | | | | Hz, 2H), 7.03- |
| | | | | 6.99 (s, 1H), |
| | | | | 4.55-4.48 (d, J = |
| | | | | 12.0 Hz, 4H). |
| 28 | 7-Bromo-1-((5- (trifluoromethyl)-1H- pyrrol-3- yl)sulfonyl)indoline | 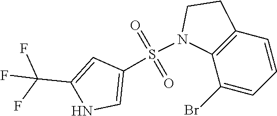 [SMILES: O=S(=O)(c1c[nH]c(C(F)(F)F)c1)N1CCc2cccc(Br)c21] | 394.9, 396.9 | ¹H NMR (400 MHz, Methanol- d₄) δ 7.53-7.46 (m, 1H), 7.32- 7.14 (m, 3H), 6.55 (d, J = 5.6 Hz, 1H), 4.06 (t, |
| | | | | J = 7.3 Hz, 2H), |
| | | | | 2.49 (t, J = 7.3 |
| | | | | Hz, 2H). |
| 29 | 1-((5- (Trifluoromethyl)-1H- pyrrol-3- yl)sulfonyl)indoline | 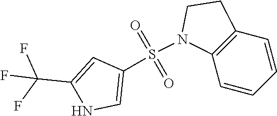 [SMILES: O=S(=O)(c1c[nH]c(C(F)(F)F)c1)N1CCc2ccccc21] | 317.1 | ¹H NMR (400 MHz, DMSO- d₆) δ 13.14 (s, 1H), 7.73 (t, J = 2.2 Hz, 1H), 7.44 (d, J = 8.2 Hz, 1H), 7.18 (t, |
| | | | | J = 7.3 Hz, 2H), |
| | | | | 6.98 (t, J = 7.4 |
| | | | | Hz, 1H), 6.89 (s, |
| | | | | 1H), 3.86 (t, J = |
| | | | | 8.4 Hz, 2H), |
| | | | | 2.97 (t, J = 8.4 |
| | | | | Hz, 2H). |
| 30 | 4-(2-Chlorophenyl)-1- ((5-(trifluoromethyl)- 1H-pyrrol-3- yl)sulfonyl)piperidine | 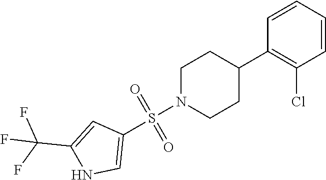 [SMILES: O=S(=O)(c1c[nH]c(C(F)(F)F)c1)N1CCC(c2ccccc2Cl)CC1] | 393.0 | ¹H NMR (400 MHz, DMSO- d₆) δ 13.17 (s, 1H), 7.67 (t, J = 2.2 Hz, 1H), 7.40 (td, J = 7.6, 1.6 Hz, 2H), 7.32 (td, J = 7.5, 1.4 Hz, 1H), 7.24 (td, J = 7.5, 1.7 Hz, 1H), |
| | | | | 6.95 (s, 1H), |
| | | | | 3.74 (d, J = 11.4 |
| | | | | Hz, 2H), 2.91 |
| | | | | (tt, J = 12.0, 3.6 |
| | | | | Hz, 1H), 2.35 |
| | | | | (td, J = 12.0, 2.5 |
| | | | | Hz, 2H), 1.84 |
| | | | | (d, J = 11.4 Hz, |
| | | | | 2H), 1.73 (qd, J = |
| | | | | 12.4, 3.9 Hz, |
| | | | | 2H) |
| 31 | 4-(5-Chloro-2- methylphenyl)-1-((5- (trifluoromethyl)-1H- pyrrol-3- yl)sulfonyl)piperidine | 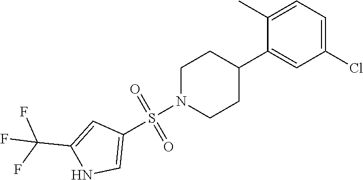 [SMILES: Cc1ccc(Cl)cc1C1CCN(S(=O)(=O)c2c[nH]c(C(F)(F)F)c2)CC1] | 407.0 | ¹H NMR (400 MHz, DMSO- d₆) δ 13.16 (s, 1H), 7.67 (d, J = 2.4 Hz, 1H), 7.21 (d, J = 1.9 Hz, 1H), 7.15 (d, J = 2.2 Hz, 2H), 6.94 (s, 1H), 3.72 (d, J = 11.4 Hz, 2H), |
| | | | | 2.72 (s, 1H), |
| | | | | 2.37 (td, J = |
| | | | | 11.7, 2.9 Hz, |
| | | | | 2H), 2.23 (s, |
| | | | | 3H), 1.79-1.61 |
| | | | | (m, 4H). |
| 32 | 4-Benzhydryl-1-((5- (trifluoromethyl)-1H- pyrrol-3- yl)sulfonyl)piperidine | 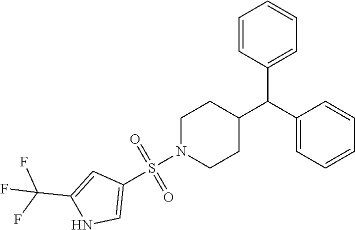 [SMILES: O=S(=O)(c1c[nH]c(C(F)(F)F)c1)N1CCC(C(c2ccccc2)c2ccccc2)CC1] | 449.1 | |
| 33 | 4-((3-(2- Chlorophenyl)pyrrolid in-1-yl)sulfonyl)-2- (trifluoromethyl)-1H- pyrrole | 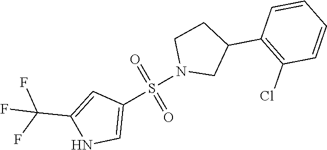 [SMILES: O=S(=O)(c1c[nH]c(C(F)(F)F)c1)N1CCC(c2ccccc2Cl)C1] | 379.1 | ¹H NMR (400 MHz, DMSO- d₆) δ 13.14 (s, 1H), 7.75 (d, J = 1.6 Hz, 1H), 7.46-7.39 (m, 1H), 7.28 (s, 2H), 7.31-7.22 (m, 1H), 7.04 (s, |
| | | | | 1H), 3.62 (dd, J = |
| | | | | 10.4, 7.4 Hz, |
| | | | | 1H), 3.47-3.36 |
| | | | | (m, 2H), 3.31- |
| | | | | 3.22 (m, 1H), |
| | | | | 3.10 (dd, J = |
| | | | | 10.4, 8.1 Hz, |
| | | | | 1H), 2.12 (dq, J = |
| | | | | 11.5, 6.7 Hz, |
| | | | | 1H), 1.96-1.82 |
| | | | | (m, 1H). |
| 34 | Trans-tert-butyl (4-(4- fluorophenyl)-1-((5- (trifluoromethyl)-1H- pyrrol-3- yl)sulfonyl)pyrrolidin- 3-yl)carbamate | 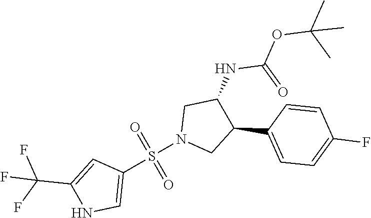 [SMILES: CC(C)(C)OC(=O)N[C@H]1CN(S(=O)(=O)c2c[nH]c(C(F)(F)F)c2)C[C@@H]1c1ccc(F)cc1] | 422.1 (M − tBu + H)⁺ | ¹H NMR (400 MHz, DMSO- d₆) δ 13.18- 13.13 (s, 1H), 7.78-7.73 (s, 1H), 7.28-7.20 (m, 2H), 7.19- 7.07 (m, 3H), 7.07-7.03 (s, 1H), 3.98-3.89 (m, 1H), 3.71- 3.62 (m, 1H), 3.61-3.52 (t, J = |
| | | | | 8.8 Hz, 1H), |
| | | | | 3.20-3.07 (m, |
| | | | | 2H), 2.95-2.86 |
| | | | | (t, J = 9.1 Hz, |
| | | | | 1H), 1.32-1.27 |
| | | | | (s, 7H), 1.23- |
| | | | | 1.14 (br.s, 2H). |
| 35 | 4-((3- Benzhydrylazetidin-1- yl)sulfonyl)-2- (trifluoromethyl)-1H- pyrrole | 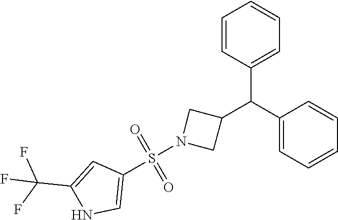 [SMILES: O=S(=O)(c1c[nH]c(C(F)(F)F)c1)N1CC(C(c2ccccc2)c2ccccc2)C1] | 421.1 | |
| 36 | 1-Phenyl-4-((5- (trifluoromethyl)-1H- pyrrol-3-yl)sulfonyl)- 1,4-diazepane | 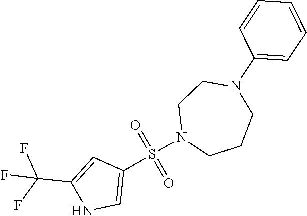 [SMILES: O=S(=O)(c1c[nH]c(C(F)(F)F)c1)N1CCCN(c2ccccc2)CC1] | 374.1 | ¹H NMR (400 MHz, DMSO- d₆) δ 13.35 (s, 1H), 7.14 (dd, J = 8.7, 7.1 Hz, 2H), 6.75-6.64 (m, 4H), 6.59 (t, J = 7.2 Hz, 1H), 3.62 (t, J = 5.3 Hz, 2H), 3.52 (t, J = 6.1 Hz, 2H), 3.39 (t, J = 5.3 Hz, 2H), 3.20- |
| | | | | 3.13 (m, 2H), |
| | | | | 1.89 (m, 2H). |
| 37 | 1-(Bis(4- fluorophenyl)methyl)- 4-((5- (trifluoromethyl)-1H- pyrrol-3- yl)sulfonyl)piperazine | 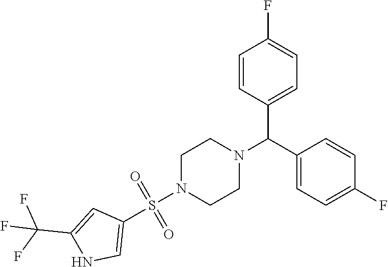 [SMILES: O=S(=O)(c1c[nH]c(C(F)(F)F)c1)N1CCN(C(c2ccc(F)cc2)c2ccc(F)cc2)CC1] | 486.2 | ¹H NMR (400 MHz, DMSO- d₆) δ 13.28- 13.24 (s, 1H), 7.70-7.64 (s, 1H), 7.49-7.45 (br s, 4H), 7.19- 7.14 (br s, 4H), 6.95-6.90 (s, 1H), 2.99- 2.73 (br s, 4H).^{†} |
| 38 | 1-((4- Chlorophenyl)(phenyl) methyl)-4-((5- (trifluoromethyl)-1H- pyrrol-3- yl)sulfonyl)piperazine | 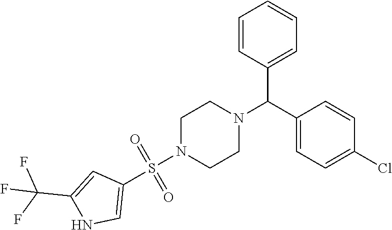 [SMILES: O=S(=O)(c1c[nH]c(C(F)(F)F)c1)N1CCN(C(c2ccccc2)c2ccc(Cl)cc2)CC1] | 484.1 | ¹H NMR (400 MHz, DMSO- d₆) δ 13.29- 13.24 (br s, 1H), 7.70-7.65 (s, 1H), 7.59-7.04 (m, 9H), 6.95- 6.90 (s, 1H), 2.92-2.78 (br s, 4H).^{†} |
| 39 | 1-(Bis(4- chlorophenyl)methyl)- 4-((5- (trifluoromethyl)-1H- pyrrol-3- yl)sulfonyl)piperazine | 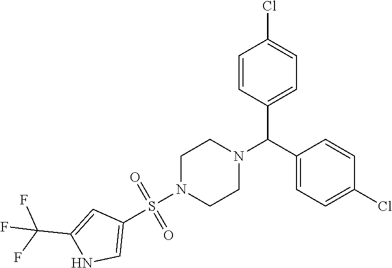 [SMILES: O=S(=O)(c1c[nH]c(C(F)(F)F)c1)N1CCN(C(c2ccc(Cl)cc2)c2ccc(Cl)cc2)CC1] | 518.1 | ¹H NMR (400 MHz, DMSO- d₆) δ 13.27- 13.22 (br.s, 1H), 7.69-7.64 (s, 1H), 7.46-7.34 (m, 8H), 6.94- 6.89 (s, 1H), 2.90-2.81 (br.s, 4H).^{†} |
| 40 | 1-((2- Chlorophenyl)(phenyl) methyl)-4-((5- (trifluoromethyl)-1H- pyrrol-3- yl)sulfonyl)piperazine | 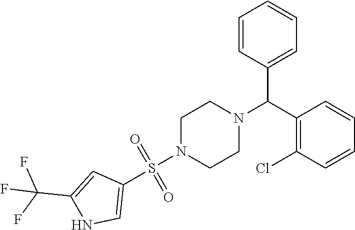 [SMILES: O=S(=O)(c1c[nH]c(C(F)(F)F)c1)N1CCN(C(c2ccccc2)c2ccccc2Cl)CC1] | 484.1 | ¹H NMR (400 MHz, DMSO- d₆) δ 13.25 (br.s, 1H), 7.78 (br.s, 1H), 7.69-7.63 (m, 1H), 7.46- 7.14 (m, 8H), 6.92 (br.s, 1H), 2.89 (br.s, 4H).^{†} |
| 41 | 1-(Phenyl(pyridin-2- yl)methyl)-4-((5- (trifluoromethyl)-1H- pyrrol-3- yl)sulfonyl)piperazine | 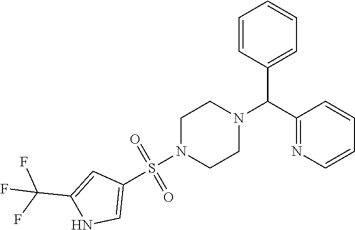 [SMILES: O=S(=O)(c1c[nH]c(C(F)(F)F)c1)N1CCN(C(c2ccccc2)c2ccccn2)CC1] | 451.2 | ¹H NMR (400 MHz, DMSO- d₆) δ 13.37- 13.32 (br s, 1H), 8.67-8.62 (d, J = 4.9 Hz, 1H), 7.90-7.83 (t, J = 7.7 Hz, 1H), 7.74-7.69 (dd, J = 3.1, 1.7 Hz, 1H), 7.56-7.48 (t, J = 7.3 Hz, 3H), 7.44-7.31 (m, 4H), 6.98- |
| | | | | 6.93 (br s, 1H), |
| | | | | 5.40-5.36 (br |
| | | | | s, 1H), 3.19- |
| | | | | 2.81 (m, 8H). |

^{†}Incomplete ¹H NMR chemical shift and integration assignments due to overlap with solvent and/or water peak.

### Example 42. 1-(S-Bromo-2-methylphenyl)-4-((5-(trifluoromethyl)-1H-pyrrol-3-yl)sulfonyl)piperazine

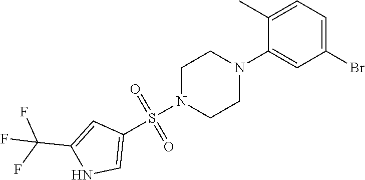 [SMILES: Cc1ccc(Br)cc1N1CCN(S(=O)(=O)c2c[nH]c(C(F)(F)F)c2)CC1]

### Step 1. 1-(5-Bromo-2-methylphenyl)piperazine

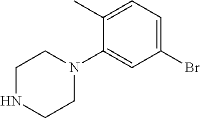

In a 250 round bottom flask a mixture of 5-bromo-2-methylaniline (1.00 g, 5.37 mmol), bis(2-chloroethyl)amine hydrochloride (0.959 g, 5.37 mmol), potassium carbonate (0.743 g, 5.37 mmol), and potassium iodide (0.892 g, 5.37 mmol) in xylenes (20 mL) was stirred at 135° C. for 10 hours. After cooling to room temperature, the solvent was removed in vacuo. The concentrate was partitioned between ethyl acetate (120 mL) and water (60 mL). The organic layer was washed with brine (30 mL), dried over Na₂SO₄, filtered and concentrated. The concentrate was taken up in a mixture of methanol (4 mL), dichloromethane (10 mL) and ethyl acetate (25 mL). A precipitate formed and was collected. The filter cake was washed with dichloromethane (3 mL) and air dried under vacuum. 1-(5-bromo-2-methylphenyl)piperazine hydrochloride (350 mg, 1.20 mmol, 22.3%) was obtained as a pale yellow solid. LC-MS calculated for C₁₁H₁₆BrN₂ (M+H)⁺: m/z=255.04, 257.04. found 255.1, 257.1. ¹H NMR (400 MHz, DMSO-d₆) δ 9.10-8.76 (s, 2H), 7.25-7.05 (m, 3H), 3.25-3.16 (t, J=5.0 Hz, 4H), 3.09-3.02 (t, J=5.0 Hz, 4H), 2.26-2.20 (d, J=6.6 Hz, 3H).

### Step 2. 1-(5-Bromo-2-methylphenyl)-4-((5-(trifluoromethyl)-1H-pyrrol-3-yl)sulfonyl)piperazine

A mixture of 5-(trifluoromethyl)-1H-pyrrole-3-sulfonyl chloride (35.8 mg, 0.153 mmol), and 1-(5-bromo-2-methylphenyl)piperazine hydrochloride salt (46.9 mg, 0.184 mmol) in dichloromethane (1 mL) at room temperature was treated with triethylamine (42.7 μL, 0.307 mmol). The resultant solution was stirred for 2 hours and then concentrated in vacuo. The concentrate was dissolved in a mixture of DMSO (0.4 mL) and acetonitrile (4.2 mL). The mixture was filtered through a 45 micron disc and then purified by preparative reverse-phase HPLC (pH=2) to afford 1-(5-bromo-2-methylphenyl)-4-((5-(trifluoromethyl)-1H-pyrrol-3-yl)sulfonyl)piperazine (30 mg, 0.066 mmol, 43%) as a white solid. LC-MS calculated for C₁₆H₁₈BrF₃N₃O₂S (M+H)⁺: m/z=452.02, 454.02. found 452.0, 454.0. ¹H NMR (400 MHz, Methanol-d₄) δ 7.55-7.50 (d, J=1.7 Hz, 1H), 7.19-7.17 (d, J=2.0 Hz, 1H), 7.15-7.11 (dd, J=8.1, 2.0 Hz, 1H), 7.09-7.04 (d, J=8.1 Hz, 1H), 6.93-6.87 (t, J=1.5 Hz, 1H), 3.18-3.11 (t, J=4.8 Hz, 4H), 3.03-2.96 (t, J=4.8 Hz, 4H), 2.21-2.17 (s, 3H).

### Example 43. 6-Bromo-2-((5-(trifluoromethyl)-1H-pyrrol-3-yl)sulfonyl)-1,2,3,4-tetrahydroisoquinoline

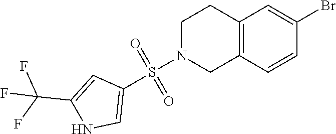 [SMILES: O=S(=O)(c1c[nH]c(C(F)(F)F)c1)N1CCc2cc(Br)ccc2C1]

### Step 1. 6-Bromo-1,2,3,4-tetrahydroisoquinoline

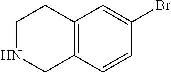 [SMILES: Brc1ccc2c(c1)CCNC2]

To a solution of tert-butyl 6-bromo-3,4-dihydroisoquinoline-2(1H)-carboxylate (100 mg, 0.320 mmol) in dichloromethane (3 mL) was added trifluoroacetic acid (490 μL, 6.41 mmol). The reaction mixture was allowed to stir at room temperature for 24 hours and then was concentrated to give 60.7 mg (0.186 mmol, 58.1%) of the desired product as the TFA salt which was used without further purification. LC-MS calculated for C₉H₁₁BrN (M+H)⁺: m/z=212.0, 214.0. found 212.0, 214.0.

### Step 2. 6-Bromo-2-((5-(trifluoromethyl)-1H-pyrrol-3-yl)sulfonyl)-1,2,3,4-tetrahydroisoquinoline

To a solution of 6-bromo-1,2,3,4-tetrahydroisoquinoline TFA salt (20.4 mg, 0.063 mmol) in dichloromethane (626 μL) was added triethylamine (52.3 μL, 0.375 mmol) and 5-(trifluoromethyl)-1H-pyrrole-3-sulfonyl chloride (15.7 mg, 0.067 mmol). The mixture was allowed to stir at room temperature for 8 hours. The crude material was taken up in acetonitrile (4 mL) and purified by preparative reverse-phase HPLC (pH=2) to give 10.5 mg (0.026 mmol, 41%) of the desired product. LC-MS calculated for C₁₄H₁₃BrF₃N₂O₂S (M+H)⁺: m/z=410.9, 408.9. found 411.0, 409.0. ¹H NMR (400 MHz, DMSO-d₆) δ 13.14 (s, 1H), 7.70 (d, J=1.7 Hz, 1H), 7.39-7.31 (m, 2H), 7.13 (d, J=8.1 Hz, 1H), 6.96 (d, J=1.5 Hz, 1H), 4.12 (s, 2H), 3.22 (t, J=6.0 Hz, 2H), 2.88 (t, J=6.0 Hz, 2H).

### Example 44. 7-Bromo-2-((5-(trifluoromethyl)-1H-pyrrol-3-yl)sulfonyl)-1,2,3,4-tetrahydroisoquinoline

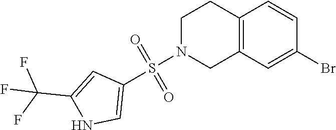 [SMILES: O=S(=O)(c1c[nH]c(C(F)(F)F)c1)N1CCc2ccc(Br)cc2C1]

The title compound was prepared according to the procedures described in Example 43, using appropriately substituted starting materials. LC-MS calculated for C₁₄H₁₃BrF₃N₂O₂S (M+H)⁺: m/z=410.9, 408.9. found 411.0, 409.0. ¹H NMR (400 MHz, DMSO-d₆) δ 13.14 (s, 1H), 7.68 (d, J=1.8 Hz, 1H), 7.41 (d, J=2.1 Hz, 1H), 7.34 (dd, J=8.2, 2.1 Hz, 1H), 7.10 (d, J=8.2 Hz, 1H), 6.94 (s, 1H), 4.17 (s, 2H), 3.23 (t, J=6.0 Hz, 2H), 2.83 (t, J=6.0 Hz, 2H).

### Example 45. 3-Chloro-N-(4-fluorophenyl)-4-(4-((5-(trifluoromethyl)-1H-pyrrol-3-yl)sulfonyl)piperazin-1-yl)benzamide

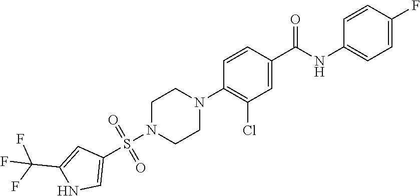 [SMILES: O=C(Nc1ccc(F)cc1)c1ccc(N2CCN(S(=O)(=O)c3c[nH]c(C(F)(F)F)c3)CC2)c(Cl)c1]

### Step 1. Methyl 3-chloro-4-(4-((5-(trifluoromethyl)-1H-pyrrol-3-yl)sulfonyl)piperazin-1-yl)benzoate

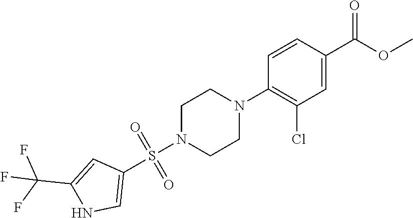 [SMILES: COC(=O)c1ccc(N2CCN(S(=O)(=O)c3c[nH]c(C(F)(F)F)c3)CC2)c(Cl)c1]

A mixture of 5-(trifluoromethyl)-1H-pyrrole-3-sulfonyl chloride (41 mg, 0.176 mmol), methyl 3-chloro-4-(piperazin-1-yl)benzoate (53.6 mg, 0.211 mmol), and triethylamine (0.049 mL, 0.351 mmol) in dichloromethane (1 mL) was stirred at room temperature for 2 h. The solvents were evaporated in vacuo and the residue purified on silica gel (12 g, 5-40% ethyl acetate in hexanes) to give the desired product (70 mg, 88%). LCMS calculated for C₁₇H₁₈ClF₃N₃O₄S (M+H)⁺: m/z=452.1; found: 452.1. ¹H NMR (400 MHz, DMSO-d₆) δ 13.24 (s, 1H), 7.89 (s, 1H), 7.91-7.84 (m, 1H), 7.71 (d, J=1.7 Hz, 1H), 7.27 (d, J=8.3 Hz, 1H), 6.97 (s, 1H), 3.83 (s, 3H), 3.23-3.16 (m, 4H), 3.04 (t, J=4.7 Hz, 4H).

### Step 2. 3-Chloro-4-(4-((5-(trifluoromethyl)-1H-pyrrol-3-yl)sulfonyl)piperazin-1-yl)benzoic Acid

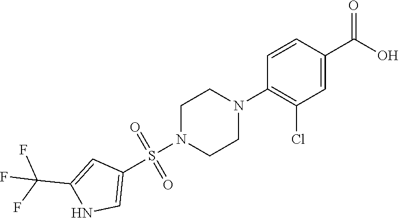 [SMILES: O=C(O)c1ccc(N2CCN(S(=O)(=O)c3c[nH]c(C(F)(F)F)c3)CC2)c(Cl)c1]

To a solution of methyl 3-chloro-4-(4-((5-(trifluoromethyl)-1H-pyrrol-3-yl)sulfonyl)piperazin-1-yl)benzoate (66 mg, 0.146 mmol) stirring in a mixture of THF (0.10 mL), methanol (0.40 mL) and water (50 μL) was added lithium hydroxide monohydrate (30.6 mg, 0.730 mmol). The resulting mixture was stirred at room temperature for 6 h. The solvents were mostly evaporated in vacuo to give a residue which was treated with water. Acidification with 1N HCl (aq.) generated a solid which was collected by filtration, washed with water, and dried to give 56 mg of the desired product. LC-MS calculated for C₁₆H₁₆ClF₃N₃O₄S (M+H)⁺: m/z=438.0. found 438.0.

### Step 3. 3-Chloro-N-(4-fluorophenyl)-4-(4-((5-(trifluoromethyl)-1H-pyrrol-3-yl)sulfonyl)piperazin-1-yl)benzamide

To a mixture of 3-chloro-4-(4-((5-(trifluoromethyl)-1H-pyrrol-3-yl)sulfonyl)piperazin-1-yl)benzoic acid (11 mg, 0.025 mmol), 4-fluoroaniline (2.89 μL, 0.030 mmol) and triethylamine (5.25 μL, 0.038 mmol) in DMF (0.20 mL) was added HATU (11.46 mg, 0.030 mmol). The resulting mixture was stirred overnight. The mixture was diluted with acetonitrile then purified by preparative reverse-phase HPLC (pH=2) to give the desired product (2.7 mg, 20%). LC-MS calculated for C₂₂H₂₀ClF₄N₄O₃S (M+H)⁺: m/z=531.1. found 531.1.

### Example 46. 1-(2-(1-Methyl-1H-pyrazol-4-yl)phenyl)-4-((5-(trifluoromethyl)-1H-pyrrol-3-yl)sulfonyl)piperazine

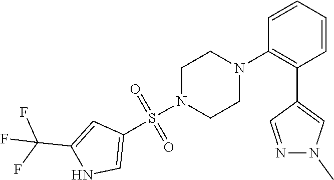 [SMILES: Cn1cc(-c2ccccc2N2CCN(S(=O)(=O)c3c[nH]c(C(F)(F)F)c3)CC2)cn1]

### Step 1. 1-(2-Bromophenyl)-4-((5-(trifluoromethyl)-1H-pyrrol-3-yl)sulfonyl)piperazine

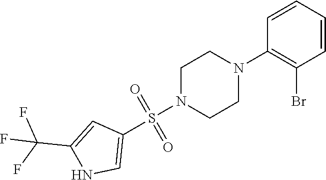 [SMILES: O=S(=O)(c1c[nH]c(C(F)(F)F)c1)N1CCN(c2ccccc2Br)CC1]

A mixture of 5-(trifluoromethyl)-1H-pyrrole-3-sulfonyl chloride (40 mg, 0.171 mmol), 1-(2-bromophenyl)piperazine (49.5 mg, 0.205 mmol), and triethylamine (0.048 mL, 0.342 mmol) in dichloromethane (0.90 mL) was stirred at room temperature for 2 h. The solvents were evaporated in vacuo and the residue purified on silica gel (12 g, 5-40% ethyl acetate in hexanes) to give the desired product (62 mg, 83%) as a solid. LC-MS calculated for C₁₅H₁₆BrF₃N₃O₂S (M+H)⁺: m/z=438.0, 440.0. found 438.0, 440.0.

### Step 2. 1-(2-(1-Methyl-JH-pyrazol-4-yl)phenyl)-4-((5-(trifluoromethyl)-1H-pyrrol-3-yl)sulfonyl)piperazine

Into a small vial were weighed 1-(2-bromophenyl)-4-((5-(trifluoromethyl)-1H-pyrrol-3-yl)sulfonyl)piperazine (13 mg, 0.030 mmol), 1-methyl-4-(4,4,5,5-tetramethyl-1,3,2-dioxaborolan-2-yl)-1H-pyrazole (9.26 mg, 0.044 mmol), chloro(2-dicyclohexylphosphino-2′,4′,6′-triisopropyl-1,1′-biphenyl)[2-(2′-amino-1,1′-biphenyl)]palladium(II) (2.33 mg, 2.97 μmol) and potassium phosphate tribasic (18.9 mg, 0.089 mmol). 1,4-Dioxane (0.40 mL) and water (0.080 mL) were added, the vial purged with N₂ then heated to 80° C. After 3 h, the mixture was cooled to room temperature, then filtered through a Si-thiol cartridge, eluting with a mixture of methanol and acetonitrile. The eluent was purified by preparative reverse-phase HPLC (pH=2) to give the desired product (4.0 mg, 30%). LC-MS calculated for C₁₉H₂₁F₃N₅O₂S (M+H)⁺: m/z=440.1. found 440.2. ¹H NMR (400 MHz, DMSO-d₆) δ 13.52 (s, 1H), 8.04 (s, 1H), 7.80 (s, 1H), 7.37 (dd, J=7.6, 1.7 Hz, 1H), 7.23-7.12 (m, 1H), 7.11-7.02 (m, 2H), 6.88-6.77 (m, 2H), 3.79 (s, 3H), 3.14-3.07 (m, 4H), 2.85 (t, J=4.9 Hz, 4H).

### Example 47. 1-(2-Methyl-5-(pyridin-4-yl)phenyl)-4-((5-(trifluoromethyl)-1H-pyrrol-3-yl)sulfonyl)piperazine

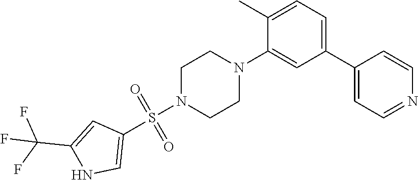 [SMILES: Cc1ccc(-c2ccncc2)cc1N1CCN(S(=O)(=O)c2c[nH]c(C(F)(F)F)c2)CC1]

The title compound was prepared according to the procedures described in Example 46, using appropriately substituted starting materials. LC-MS calculated for C₂₁H₂₂F₃N₄O₂S (M+H)⁺: m/z=451.1. found 451.1. ¹H NMR (400 MHz, Methanol-d₄) δ 8.75 (m, 2H), 8.21 (d, J=5.9 Hz, 2H), 7.58 (d, J=6.6 Hz, 2H), 7.54 (d, J=1.7 Hz, 1H), 7.42 (d, J=8.2 Hz, 1H), 6.92 (s, 1H), 3.24-3.09 (m, 8H), 2.35 (s, 3H).

### Example 48. 5-(2-Fluoro-4-methylphenyl)-1-((5-(trifluoromethyl)-1H-pyrrol-3-yl)sulfonyl)-1,2,3,4-tetrahydroquinoline

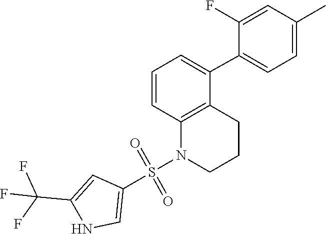 [SMILES: Cc1ccc(-c2cccc3c2CCCN3S(=O)(=O)c2c[nH]c(C(F)(F)F)c2)c(F)c1]

### Step 1. tert-Butyl 5-(2-fluoro-4-methylphenyl)-3,4-dihydroquinoline-1(2H)-carboxylate

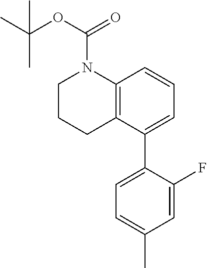 [SMILES: Cc1ccc(-c2cccc3c2CCCN3C(=O)OC(C)(C)C)c(F)c1]

To a solution of tert-butyl 5-bromo-3,4-dihydroquinoline-1(2H)-carboxylate (37.5 mg, 0.120 mmol) in 1,4-dioxane (1001 μL) and water (200 μL), were added 2-(2-fluoro-4-methylphenyl)-4,4,5,5-tetramethyl-1,3,2-dioxaborolane (36.7 mg, 0.155 mmol), potassium phosphate (80.9 mg, 0.381 mmol), and (1,1′-bis(diphenylphosphino)ferrocene)-dichloropalladium(II) (17.3 mg, 0.021 mmol). The solution was allowed to stir at room temperature and was degassed with N₂ for 5 minutes. The reaction vessel was sealed and heated at 80° C. After 20 hours, the reaction mixture was cooled to room temperature, diluted with dichloromethane (3 mL) and then passed through a phase separator. The organic layer was collected and filtered through a thiol cartridge. The filtrate was concentrated and purified by preparative reverse-phase HPLC (pH=2) to give 31.6 mg (0.093 mmol, 77%) of the desired product. LC-MS calculated for C₂₁H₂₅FNO₂ (M+H)⁺: m/z=342.2; found (M-tBu+H)⁺: 286.1.

### Step 2. 5-(2-Fluoro-4-methylphenyl)-1,2,3,4-tetrahydroquinoline

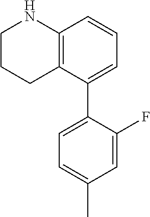 [SMILES: Cc1ccc(-c2cccc3c2CCCN3)c(F)c1]

To a solution containing tert-butyl 5-(2-fluoro-4-methylphenyl)-3,4-dihydroquinoline-1(2H)-carboxylate (31.6 mg, 0.093 mmol) in dichloromethane (926 μL) was added trifluoroacetic acid (142 μL, 1.851 mmol). The reaction mixture was stirred at room temperature for 24 hours and then concentrated to give 44.8 mg of the desired product containing residual trifluoroacetic acid which was used without further purification. LC-MS calculated for C₁₆H₁₇FN (M+H)⁺: m/z=242.1. found 242.1.

### Step 3. 5-(2-Fluoro-4-methylphenyl)-1-((5-(trifluoromethyl)-1H-pyrrol-3-yl)sulfonyl)-1,2,3,4-tetrahydroquinoline

To a solution containing 5-(2-fluoro-4-methylphenyl)-1,2,3,4-tetrahydroquinoline TFA salt (32.9 mg, 0.093 mmol) in dichloromethane (926 μL) were added 5-(trifluoromethyl)-1H-pyrrole-3-sulfonyl chloride (27.0 mg, 0.116 mmol) and triethylamine (38.7 μL, 0.278 mmol). The reaction mixture was stirred at room temperature for 8 hours and then quenched with water (0.5 mL) and filtered using a phase separator. The organic layer was concentrated in vacuo and purified by preparative reverse-phase HPLC (pH=2) to give 8.6 mg of the desired product (0.020 mmol, 21%). LC-MS calculated for C₂₁H₁₉F₄N₂O₂S (M+H)⁺: m/z=439.1. found 439.1. ¹H NMR (400 MHz, DMSO-d₆) δ 13.08 (s, 1H), 7.64 (dd, J=8.3, 1.2 Hz, 1H), 7.58 (dd, J=3.2, 1.7 Hz, 1H), 7.28 (t, J=7.9 Hz, 1H), 7.08-6.98 (m, 4H), 6.56 (dt, J=2.6, 1.4 Hz, 1H), 3.71 (s, 2H), 2.35 (s, 3H), 2.08 (t, J=6.5 Hz, 2H), 1.72-1.56 (m, 2H).

### Examples 49-53

Examples 49-53 in Table 2 were prepared according to the procedures described in Example 48, using appropriately substituted starting materials.

TABLE 2

| Ex. | | | LCMS | |
|---|---|---|---|---|
| No. | Name | Structure | [M + H]⁺ | ¹H NMR |
| 49 | N,N-Dimethyl-4-(1- ((5-(trifluoromethyl)- 1H-pyrrol-3- yl)sulfonyl)-1,2,3,4- tetrahydroquinolin-5- yl)aniline | 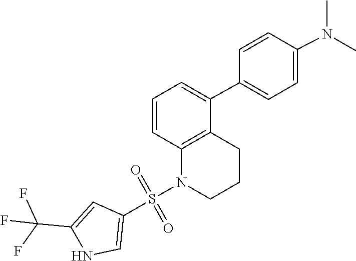 [SMILES: CN(C)c1ccc(-c2cccc3c2CCCN3S(=O)(=O)c2c[nH]c(C(F)(F)F)c2)cc1] | 450.2 | ¹H NMR (400 MHz, Methanol-d₄) δ 7.62 (dd, J = 8.1, 1.3 Hz, 1H), 7.28 − 7.19 (m, 2H), 7.07 (dd, J = 7.6, 1.3 Hz, 1H), 7.05 − 6.98 (m, 2H), 6.83 − 6.75 (m, 2H), 6.43 (m, 1H), 3.78 − 3.73 (m, 2H), 2.96 (s, 6H), 2.23 − 2.15 (m, 2H), 1.67 (m, 2H). |
| 50 | 5-(Chroman-6-yl)-1- ((5-(trifluoromethyl)- 1H-pyrrol-3- yl)sulfonyl)-1,2,3,4- tetrahydroquinoline | 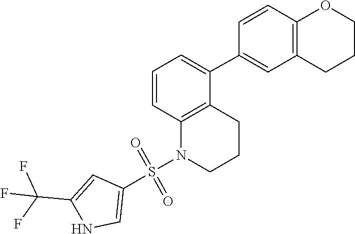 [SMILES: O=S(=O)(c1c[nH]c(C(F)(F)F)c1)N1CCCc2c(-c3ccc4c(c3)CCCO4)cccc21] | 463.1 | ¹H NMR (400 MHz, DMSO-d₆) δ 13.10 (s, 1H), 7.60 − 7.52 (m, 2H), 7.23 (t, J = 7.9 Hz, 1H), 7.00 (dd, J = 7.6, 1.3 Hz, 1H), 6.90 − 6.81 (m, 2H), 6.75 (d, J = 8.2 Hz, 1H), 6.52 (m, 1H), 4.18 − 4.11 (m, 2H), 3.70 (t, J = 6.3 Hz, 2H), 2.73 (t, J = 6.4 Hz, 2H), 2.15 (t, J = |
| | | | | 6.4 Hz, 2H), 1.98 − |
| | | | | 1.88 (m, 2H), 1.64 |
| | | | | (m, 2H). |
| 51 | 5-(o-Tolyl)-1-((5- (trifluoromethyl)-1H- pyrrol-3-yl)sulfonyl)- 1,2,3,4- tetrahydroquinoline | 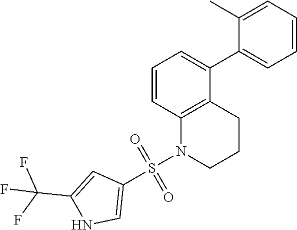 [SMILES: Cc1ccccc1-c1cccc2c1CCCN2S(=O)(=O)c1c[nH]c(C(F)(F)F)c1] | 421.1 | ¹H NMR (400 MHz, Methanol-d₄) δ 7.74 (dd, J = 8.4, 1.3 Hz, 1H), 7.33 − 7.15 (m, 5H), 6.96 (td, J = 7.1, 1.3 Hz, 2H), 6.43 (q, J = 1.4 Hz, 1H), 3.92 (ddd, J = 12.6, 7.1, 5.1 Hz, 1H), 3.68 (ddd, J= 13.0, 7.7, 4.9 Hz, 1H), 2.06 (m, 1H), 1.93 − 1.83 (m, 4H), 1.77 − |
| | | | | 1.55 (m, 2H). |
| 52 | Methyl 3-(1-((5- (trifluoromethyl)-1H- pyrrol-3-yl)sulfonyl)- 1,2,3,4- tetrahydroquinolin-5- yl)benzoate | 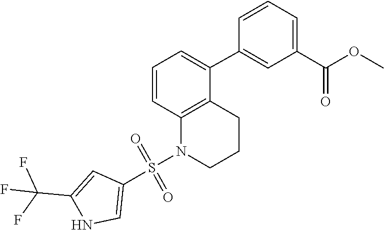 [SMILES: COC(=O)c1cccc(-c2cccc3c2CCCN3S(=O)(=O)c2c[nH]c(C(F)(F)F)c2)c1] | 465.1 | ¹H NMR (400 MHz, Methanol-d₄) δ 8.02 (dt, J = 7.9, 1.4 Hz, 1H), 7.83 (td, J = 1.8, 0.6 Hz, 1H), 7.76 (dd, J = 8.3, 1.3 Hz, 1H), 7.53 (td, J = 7.7, 0.6 Hz, 1H), 7.43 (ddd, J = 7.6, 1.8, 1.2 Hz, 1H), 7.36 − 7.27 (m, 2H), 7.10 (dd, J = 7.6, 1.3 Hz, 1H), 6.45 (dq, |
| | | | | J = 2.2, 1.2 Hz, |
| | | | | 1H), 3.92 (s, 3H), |
| | | | | 3.84 − 3.76 (m, |
| | | | | 2H), 2.18 (t, J = |
| | | | | 6.4 Hz, 2H), 1.70 |
| | | | | (m, 2H). |
| 53 | 6-Phenyl-2-((5- (trifluoromethyl)-1H- pyrrol-3-yl)sulfonyl)- 1,2,3,4- tetrahydroisoquinoline | 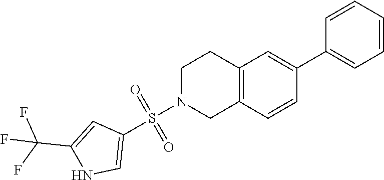 [SMILES: O=S(=O)(c1c[nH]c(C(F)(F)F)c1)N1CCc2cc(-c3ccccc3)ccc2C1] | 407.1 | ¹H NMR (400 MHz, DMSO-d₆) δ 13.15 (s, 1H), 7.73 (d, J = 1.7 Hz, 1H), 7.67 − 7.59 (m, 2H), 7.46 (ddt, J = 10.6, 5.4, 2.7 Hz, 4H), 7.39 − 7.31 (m, 1H), 7.25 (d, J = 8.0 Hz, 1H), 6.98 (t, |
| | | | | J = 1.5 Hz, 1H), |
| | | | | 4.20 (s, 2H), 3.27 |
| | | | | (t, J = 6.0 Hz, 2H), |
| | | | | 2.96 (t, J = 6.0 Hz, |
| | | | | 2H). |

### Example 54. (R)-4-(5-Chloro-2-methylphenyl)-2-methyl-1-((5-(trifluoromethyl)-1H-pyrrol-3-yl)sulfonyl)piperazine

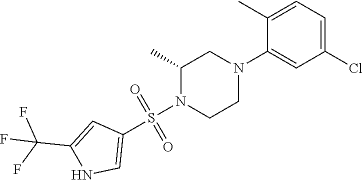 [SMILES: Cc1ccc(Cl)cc1N1CCN(S(=O)(=O)c2c[nH]c(C(F)(F)F)c2)[C@H](C)C1]

### Step 1. tert-Butyl (R)-4-(5-chloro-2-methylphenyl)-2-methylpiperazine-1-carboxylate

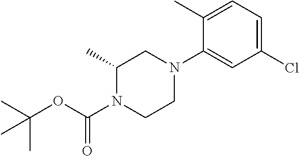 [SMILES: Cc1ccc(Cl)cc1N1CCN(C(=O)OC(C)(C)C)[C@H](C)C1]

4-Chloro-2-iodo-1-methylbenzene (151 mg, 0.599 mmol), tert-butyl (R)-2-methylpiperazine-1-carboxylate (100 mg, 0.499 mmol), BINAP (31.1 mg, 0.050 mmol), and sodium tert-butoxide (67.2 mg, 0.699 mmol) were weighed into a microwave tube. Anhydrous toluene (2.0 mL) was added. The vial was degassed with N₂ before adding Pd₂(dba)₃ (22.86 mg, 0.025 mmol). The vial was sealed and heated at 100° C. for 18 h. The reaction mixture was cooled, filtered through a pad of celite, and concentrated in vacuo. The crude material was purified on silica gel (24 g, 0-40% ethyl acetate in hexanes) to give the desired product (64 mg, 39.5%). LC-MS calculated for C₁₇H₂₆ClN₂O₂(M+H)⁺: m/z=325.1. found 325.1.

### Step 2. (R)-1-(5-Chloro-2-methylphenyl)-3-methylpiperazine

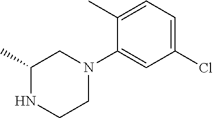

To a mixture of tert-butyl (R)-4-(5-chloro-2-methylphenyl)-2-methylpiperazine-1-carboxylate (64 mg, 0.197 mmol) in dichloromethane (0.50 mL) was added 4N HCl in 1,4-dioxane (0.345 mL, 1.379 mmol). The resulting mixture was stirred at room temperature for 2 h. The mixture was concentrated in vacuo to give the crude product as a hydrochloride salt which was used directly in the next reaction. LC-MS calculated for C₁₂H₁₈ClN₂ (M+H)⁺: m/z=225.1. found 225.2.

### Step 3. (R)-4-(5-Chloro-2-methylphenyl)-2-methyl-1-((5-(trifluoromethyl)-1H-pyrrol-3-yl)sulfonyl)piperazine

A mixture of 5-(trifluoromethyl)-1H-pyrrole-3-sulfonyl chloride (10 mg, 0.043 mmol), (R)-1-(5-chloro-2-methylphenyl)-3-methylpiperazine hydrochloride (12.3 mg, 0.047 mmol) and triethylamine (0.018 mL, 0.128 mmol) in dichloromethane (0.20 mL) was stirred at room temperature for 3 h. The mixture was concentrated, and the residue purified by preparative reverse-phase HPLC (pH=2) to afford the desired product (3.9 mg, 21.6% yield). LC-MS calculated for C₁₇H₂₀ClF₃N₃O₂S (M+H)⁺: m/z=422.1. found 422.0. ¹H NMR (400 MHz, DMSO-d₆) 13.05 (s, 1H), 7.69 (d, J=1.7 Hz, 1H), 7.18 (d, J=8.1 Hz, 1H), 7.03 (dd, J=8.1, 2.1 Hz, 1H), 6.99-6.92 (m, 2H), 4.03 (dd, J=8.7, 5.7 Hz, 1H), 3.55 (t, J=14.8 Hz, 1H), 3.25 (td, J=12.0, 3.0 Hz, 1H), 2.97 (d, J=11.6 Hz, 1H), 2.86 (d, J=11.5 Hz, 1H), 2.73 (dd, J=11.7, 3.5 Hz, 1H), 2.63 (td, J=11.6, 3.3 Hz, 1H), 2.21 (s, 3H), 1.22 (d, J=6.7 Hz, 3H).

### Examples 55-61

Examples 55-61 in Table 3 were prepared according to the procedures described in Example 54, using appropriately substituted starting materials.

TABLE 3

| Ex. | | | LCMS | |
|---|---|---|---|---|
| No. | Name | Structure | [M + H]⁺ | ¹H NMR |
| 55 | (S)-4-(5-Chloro-2- methylphenyl)-2- methyl-1-((5- (trifluoromethyl)-1H- pyrrol-3- yl)sulfonyl)piperazine | 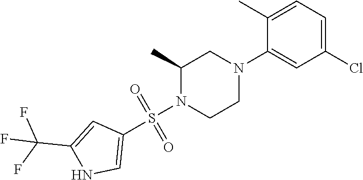 [SMILES: Cc1ccc(Cl)cc1N1CCN(S(=O)(=O)c2c[nH]c(C(F)(F)F)c2)[C@@H](C)C1] | 422.0 | ¹H NMR (400 MHz, DMSO-d₆) δ 13.05 (s, 1H), 7.69 (d, J = 1.8 Hz, 1H), 7.18 (d, J = 8.1 Hz, 1H), 7.03 (dd, J = 8.1, 2.1 Hz, 1H), 7.00 − 6.92 (m, 2H), 4.05 (d, J = 7.0 Hz, 1H), 3.57 (d, J = 12.7 Hz, |
| | | | | 1H), 3.25 (td, J = |
| | | | | 12.1, 3.0 Hz, 1H), |
| | | | | 2.97 (d, J = 11.5 |
| | | | | Hz, 1H), 2.87 (d, |
| | | | | J = 11.6 Hz, 1H), |
| | | | | 2.73 (dd, J = 11.7, |
| | | | | 3.5 Hz, 1H), 2.63 |
| | | | | (td, J = 11.6, 3.3 |
| | | | | Hz, 1H), 2.21 (s, |
| | | | | 3H), 1.22 (d, J = |
| | | | | 6.7 Hz, 3H). |
| 56 | 1-(5-Chloro-2- methylphenyl)-2- methyl-4-((5- (trifluoromethyl)-1H- pyrrol-3- yl)sulfonyl)piperazine | 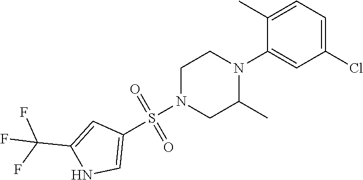 [SMILES: Cc1ccc(Cl)cc1N1CCN(S(=O)(=O)c2c[nH]c(C(F)(F)F)c2)CC1C] | 422.0 | ¹H NMR (400 MHz, DMSO-d₆) δ 13.21 (s, 1H), 7.70 (d, J = 1.7 Hz, 1H), 7.24 − 7.17 (m, 2H), 7.09 (dd, J = 8.2, 2.1 Hz, 1H), 6.96 (s, 1H), 3.43 − 3.34 (m, 1H), 3.29 − 3.23 (m, 1H), 3.04 − 2.95 (m, 1H), 2.73 (dt, J = |
| | | | | 16.5, 5.1 Hz, 2H), |
| | | | | 2.42 − 2.32 (m, |
| | | | | 1H), 2.14 (s, 3H), |
| | | | | 2.08 (s, 1H), 0.79 |
| | | | | (d, J = 6.1 Hz, 3H). |
| 57 | 4-(5-Chloro-2- methylphenyl)-2,6- dimethyl-1-((5- (trifluoromethyl)-1H- pyrrol-3- yl)sulfonyl)piperazine | 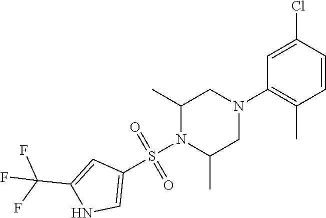 [SMILES: Cc1ccc(Cl)cc1N1CC(C)N(S(=O)(=O)c2c[nH]c(C(F)(F)F)c2)C(C)C1] | 436.1 | ¹H NMR (400 MHz, DMSO-d₆) δ 12.97 (s, 1H), 7.68 (dd, J = 3.1, 1.7 Hz, 1H), 7.19 (d, J = 8.1 Hz, 1H), 7.05 (dd, J = 8.0, 2.1 Hz, 1H), 6.99 (s, 1H), 6.88 (d, J = 2.2 Hz, 1H), 4.15 − 4.04 (m, 2H), 2.84 (d, J = 11.7 Hz, 2H), 2.52 − 2.44 (m, 2H), 2.27 (s, |
| | | | | 3H), 1.46 (d, J = |
| | | | | 6.9 Hz, 6H). |
| 58 | (4-(5-Chloro-2- methylphenyl)-1-((5- (trifluoromethyl)-1H- pyrrol-3- yl)sulfonyl)piperazin- 2-yl)methanol | 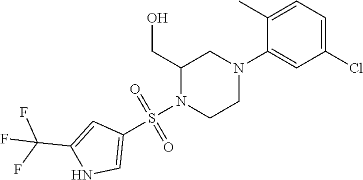 [SMILES: Cc1ccc(Cl)cc1N1CCN(S(=O)(=O)c2c[nH]c(C(F)(F)F)c2)C(CO)C1] | 438.1 | |
| 59 | (1S,4S)-2-(4-Chloro-2- methylphenyl)-5-((5- (trifluoromethyl)-1H- pyrrol-3-yl)sulfonyl)- 2,5- diazabicyclo[2.2.1] heptane | 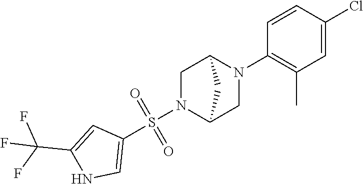 [SMILES: Cc1cc(Cl)ccc1N1C[C@@H]2C[C@H]1CN2S(=O)(=O)c1c[nH]c(C(F)(F)F)c1] | 420.1 | ¹H NMR (400 MHz, DMSO-d₆) δ 13.11 (s, 1H), 7.70 (dd, J = 3.2, 1.7 Hz, 1H), 7.08 − 6.98 (m, 2H), 6.76 (dq, J = 3.5, 2.0 Hz, 2H), 4.41 (s, 1H), 4.26 (s, 1H), 3.45 − 3.35 (m, 2H), 3.25 − 3.16 (m, 2H), 2.11 (s, |
| | | | | 3H), 1.72 (d, J = |
| | | | | 9.9 Hz, 1H), 1.16 |
| | | | | (d, J = 9.9 Hz, 1H). |
| 60 | 2-(5-Chloro-2- methylphenyl)-6-((5- (trifluoromethyl)-1H- pyrrol-3-yl)sulfonyl)- 2,6- diazaspiro[3.3]heptane | 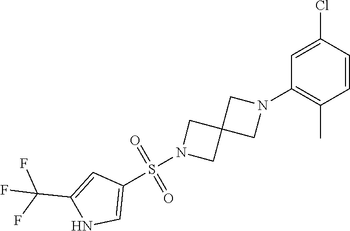 [SMILES: Cc1ccc(Cl)cc1N1CC2(C1)CN(S(=O)(=O)c1c[nH]c(C(F)(F)F)c1)C2] | 420.0 | ¹H NMR (400 MHz, DMSO-d₆) δ 13.28 (s, 1H), 7.79 (dd, J = 3.3, 1.7 Hz, 1H), 7.08 (s, 1H), 6.96 (d, J = 8.0 Hz, 1H), 6.68 (dd, J= 7.9, 2.1 Hz, 1H), 6.31 (d, J = 2.1 Hz, 1H), 3.88 (s, 4H), 3.80 (s, 4H), 2.04 (s, 3H). |
| 61 | 3-(5-Chloro-2- methylphenyl)-7-((5- (trifluoromethyl)-1H- pyrrol-3-yl)sulfonyl)- 3,7- diazabicyclo[4.2.0] octane | 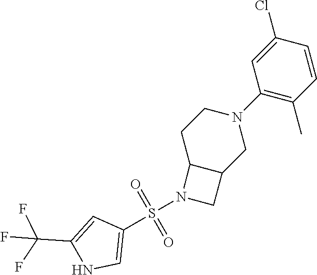 [SMILES: Cc1ccc(Cl)cc1N1CCC2C(C1)CN2S(=O)(=O)c1c[nH]c(C(F)(F)F)c1] | 434.1 | ¹H NMR (400 MHz, DMSO-d₆) δ 13.28 (s, 1H), 7.76 (dd, J = 3.2, 1.7 Hz, 1H), 7.18 (d, J = 8.1 Hz, 1H), 7.06 (d, J = 2.2 Hz, 1H), 7.00 (dd, J = 8.0, 2.2 Hz, 2H), 3.85 (dd, J = 6.7, 3.9 Hz, 1H), 3.45 − 3.32 (m, 2H), 3.28 (dd, J = 7.4, 1.7 Hz, 1H), 3.00 − 2.89 (m, 3H), 2.44 (q, J = 7.7 Hz, 1H), 2.18 (s, 3H), 2.00 − |
| | | | | 1.78 (m, 2H). |

### Example 62. (S)-2-Methyl-4-(2-methyl-5-(pyridin-3-yl)phenyl)-1-((5-(trifluoromethyl)-1H-pyrrol-3-yl)sulfonyl)piperazine

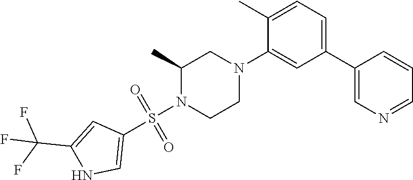 [SMILES: Cc1ccc(-c2cccnc2)cc1N1CCN(S(=O)(=O)c2c[nH]c(C(F)(F)F)c2)[C@@H](C)C1]

Into a small vial were weighed (S)-4-(5-chloro-2-methylphenyl)-2-methyl-1-((5-(trifluoromethyl)-1H-pyrrol-3-yl)sulfonyl)piperazine (Example 55) (7 mg, 0.017 mmol), 3-(4,4,5,5-tetramethyl-1,3,2-dioxaborolan-2-yl)pyridine (6.81 mg, 0.033 mmol), chloro(2-dicyclohexylphosphino-2′,4′,6′-triisopropyl-1,1′-biphenyl)[2-(2′-amino-1,1′-biphenyl)]palladium(II) (1.567 mg, 1.991 μmol) and potassium phosphate tribasic (8.81 mg, 0.041 mmol). 1,4-Dioxane (0.3 mL) and water (60 μL) were added, the vial purged with N₂, then heated to 80° C. The mixture was stirred for 17 h. After cooling, the reaction mixture was filtered through a Si-Thiol cartridge using a mixture of acetonitrile and methanol. The resulting eluent was purified by preparative reverse-phase HPLC (pH=2) to give the desired product as the TFA salt (1.2 mg, 15.2% yield). LC-MS calculated for C₂₂H₂₄F₃N₄O₂S (M+H)⁺: m/z=465.1. found 465.2.

### Example 63. 4-(Phenyl(4-((5-(trifluoromethyl)-1H-pyrrol-3-yl)sulfonyl)piperazin-1-yl)methyl)benzonitrile

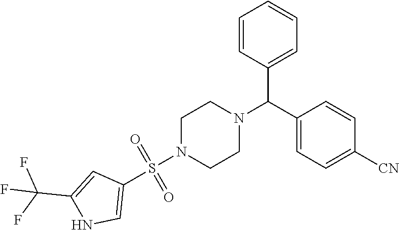 [SMILES: N#Cc1ccc(C(c2ccccc2)N2CCN(S(=O)(=O)c3c[nH]c(C(F)(F)F)c3)CC2)cc1]

### Step 1. tert-Butyl 4-((4-cyanophenyl)(phenyl)methyl)piperazine-1-carboxylate

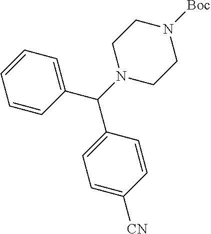 [SMILES: CC(C)(C)OC(=O)N1CCN(C(c2ccccc2)c2ccc(C#N)cc2)CC1]

To a mixture of 4-(hydroxy(phenyl)methyl)benzonitrile (400.0 mg, 1.912 mmol) in THF (6.0 mL) were added triethylamine (0.533 mL, 3.82 mmol) followed by methanesulfonyl chloride (0.30 mL, 3.8 mmol) at 0° C. The reaction mixture was warmed to room temperature and stirred for 90 min. tert-Butyl piperazine-1-carboxylate (2.30 g, 12.4 mmol) and triethylamine (0.53 mL) were then added and the reaction mixture was stirred at room temperature for three days. After concentration, the crude product was purified on silica gel (40 g, 0-35% ethyl acetate in hexanes) to afford the desired product (466 mg, 65%). LCMS calculated for C₂₃H₂₈N₃O₂ (M+H)⁺: m/z=378.2. found 378.2.

### Step 2. 4-(Phenyl(piperazin-1-yl)methyl)benzonitrile

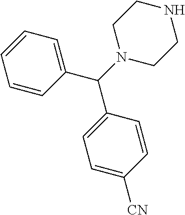 [SMILES: N#Cc1ccc(C(c2ccccc2)N2CCNCC2)cc1]

To a mixture of tert-butyl 4-((4-cyanophenyl)(phenyl)methyl)piperazine-1-carboxylate (220.0 mg, 0.58 mmol) in THF (0.5 mL) was added 4M HCl in dioxane (3.0 mL). The resulting mixture was stirred at room temperature for 30 min. The mixture was concentrated in vacuo to give the crude product as a hydrochloride salt (260 mg) which was used directly in the next reaction. LCMS calculated for C₁₈H₂₀N₃ (M+H)⁺: m/z=278.2. found 278.2.

### Step 3. 4-(Phenyl(4-((5-(trifluoromethyl)-1H-pyrrol-3-yl)sulfonyl)piperazin-1-yl)methyl)benzonitrile

To a mixture of 4-(phenyl(piperazin-1-yl)methyl)benzonitrile (120 mg, 0.433 mmol) in THF (3.2 mL) was added triethylamine (0.18 mL, 1.3 mmol) followed by 5-(trifluoromethyl)-1H-pyrrole-3-sulfonyl chloride (152 mg, 0.65 mmol). The resulting mixture was stirred at room temperature for 20 min. The mixture was diluted with water and methanol and purified by preparative reverse-phase HPLC (pH=2). The isolated material was treated with metal scavenger (SiliaPrep™ Thiol SPE cartridge) to afford the desired racemic product (173 mg, 84%). LCMS calculated for C₂₃H₂₂F₃N₄O₂S (M+H)⁺: m/z=475.1. found 475.1. ¹H NMR (500 MHz, DMSO-d₆) δ 13.27 (s, 1H), 7.78 (d, J=7.9 Hz, 2H), 7.72-7.56 (m, 3H), 7.43 (d, J=7.6 Hz, 2H), 7.33 (t, J=7.5 Hz, 2H), 7.25 (t, J=7.3 Hz, 1H), 6.92 (s, 1H), 4.64 (br s, 1H), 2.90 (br s, 4H), 2.52 (br s, partial overlap with solvent peak).

### Example 64. 1-(Bis(4-bromophenyl)methyl)-4-((5-(trifluoromethyl)-1H-pyrrol-3-yl)sulfonyl)piperazine

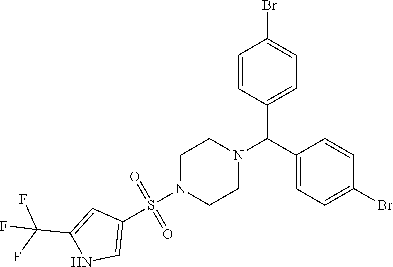 [SMILES: O=S(=O)(c1c[nH]c(C(F)(F)F)c1)N1CCN(C(c2ccc(Br)cc2)c2ccc(Br)cc2)CC1]

The title compound was prepared according to the procedures described in Example 63, using appropriately substituted starting materials. LCMS calculated for C₂₂H₂₁Br₂F₃N₃O₂S (M+H)⁺: m/z=608.0. found 608.0.

### Example 65 and Example 66. 4-(Phenyl(4-((5-(trifluoromethyl)-1H-pyrrol-3-yl)sulfonyl)piperazin-1-yl)methyl)benzonitrile, Chiral Peak 1 and Chiral Peak 2

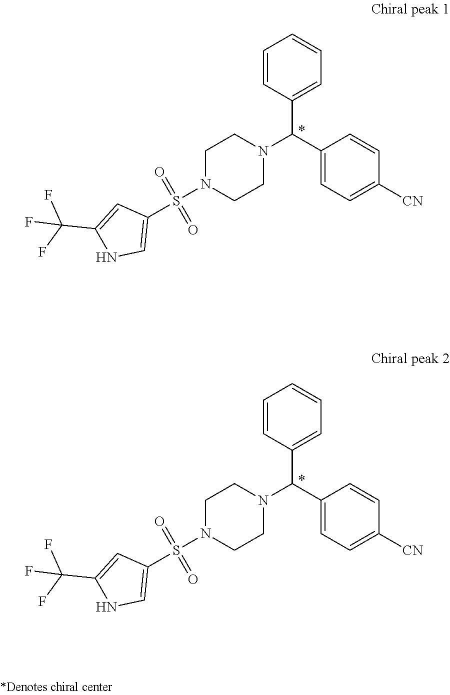 [SMILES: N#Cc1ccc(C(c2ccccc2)N2CCN(S(=O)(=O)c3c[nH]c(C(F)(F)F)c3)CC2)cc1 ; N#Cc1ccc(C(c2ccccc2)N2CCN(S(=O)(=O)c3c[nH]c(C(F)(F)F)c3)CC2)cc1]

40 mg of Example 63 was separated by chiral HPLC (Phenomenex Lux 5 μm i-Amylose-1. 21.2×250 mm column, eluting with a gradient of 30% EtOH in hexanes, at flow rate of 20 mL/min) to provide chiral peak 1(Example 65) (13.1 mg, 33%) and chiral peak 2 (Example 66) (10.2 mg, 26%) both as the free base.

Example 65: LCMS calculated for C₂₃H₂₂F₃N₄O₂S (M+H)⁺: m/z=475.1. found 475.1. ¹H NMR (500 MHz, DMSO-d₆) δ 13.22 (s, 1H), 7.78-7.71 (m, 2H), 7.65-7.59 (m, 3H), 7.41-7.36 (m, 2H), 7.29 (t, J=7.6 Hz, 2H), 7.23-7.17 (m, 1H), 6.95-6.88 (m, 1H), 4.48 (s, 1H), 2.88 (s, 4H), 2.40 (m, 4H).

Example 66: LCMS calculated for C₂₃H₂₂F₃N₄O₂S (M+H)⁺: m/z=475.1. found 475.1. ¹H NMR (500 MHz, DMSO-d₆) δ 13.22 (s, 1H), 7.79-7.71 (m, 2H), 7.68-7.57 (m, 3H), 7.42-7.35 (m, 2H), 7.29 (t, J=7.5 Hz, 2H), 7.24-7.16 (m, 1H), 6.92 (m, 1H), 4.48 (s, 1H), 2.89 (s, 4H), 2.40 (m, 4H).

### Example 67. 4-((4-Chlorophenyl)(4-((5-(trifluoromethyl)-1H-pyrrol-3-yl)sulfonyl)piperazin-1-yl)methyl)benzonitrile

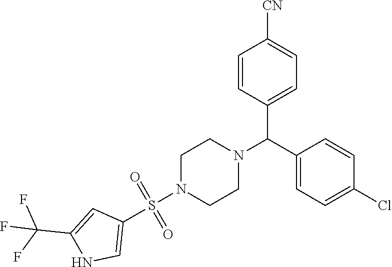 [SMILES: N#Cc1ccc(C(c2ccc(Cl)cc2)N2CCN(S(=O)(=O)c3c[nH]c(C(F)(F)F)c3)CC2)cc1]

### Step 1. 4-((4-Chlorophenyl)(piperazin-1-yl)methyl)benzonitrile

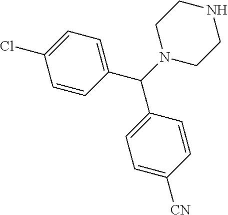 [SMILES: N#Cc1ccc(C(c2ccc(Cl)cc2)N2CCNCC2)cc1]

In a 10 mL microwave vial, a mixture of 4-(amino(4-chlorophenyl)methyl)benzonitrile (100 mg, 0.412 mmol), bis(2-chloroethyl)amine hydrochloride (81 mg, 0.453 mmol) and N,N-diisopropylethylamine (1.44 mL, 8.24 mmol) was stirred at 140° C. for 16 h. After cooling to room temperature, the mixture was treated with ca. 1:1 ethyl acetate/THF and then decanted to remove the solvents. The remaining undissolved tar-like residue was dried in vacuo to afford the desired product as a light brown powder which was used directly in the next step. LCMS calculated for C₁₈H₁₉ClN₃ (M+H)⁺: m/z=312.1. found 312.1.

### Step 2. 4-((4-Chlorophenyl)(4-((5-(trifluoromethyl)-1H-pyrrol-3-yl)sulfonyl)piperazin-1-yl)methyl)benzonitrile

To a mixture of 4-((4-chlorophenyl)(piperazin-1-yl)methyl)benzonitrile (20.0 mg, 0.064 mmol) in THF (0.40 mL) were added triethylamine (0.027 mL, 0.19 mmol) followed by 5-(trifluoromethyl)-1H-pyrrole-3-sulfonyl chloride (22.5 mg, 0.096 mmol). The resulting mixture was stirred at room temperature for 20 min. The mixture was diluted with water and methanol and purified by preparative reverse-phase HPLC (pH=2). The isolated material was treated with metal scavenger (SiliaPrep™ Thiol SPE cartridge) to afford the desired racemic product (7.4 mg, 23%). LCMS calculated for C₂₃H₂₁ClF₃N₄O₂S (M+H)⁺: m/z=509.1. found 509.1.

### Examples 68-94

Examples 68-94 in Table 4 were prepared according to the procedures described in Example 67, using appropriately substituted starting materials.

TABLE 4

| Ex. | | | LCMS | |
|---|---|---|---|---|
| No. | Name | Structure | [M + H]⁺ | ¹H NMR |
| 68 | 1-((4- Chlorophenyl)(4- fluorophenyl)methyl)- 4-((5- (trifluoromethyl)-1H- pyrrol-3- yl)sulfonyl)piperazine | 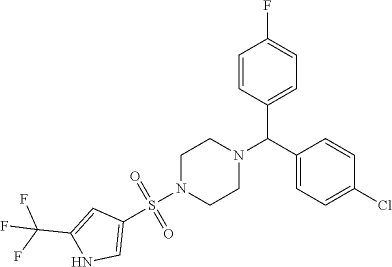 [SMILES: O=S(=O)(c1c[nH]c(C(F)(F)F)c1)N1CCN(C(c2ccc(F)cc2)c2ccc(Cl)cc2)CC1] | 502.1 | |
| 69 | 1-((4- Bromophenyl)(phenyl) methyl)-4-((5- (trifluoromethyl)-1H- pyrrol-3- yl)sulfonyl)piperazine | 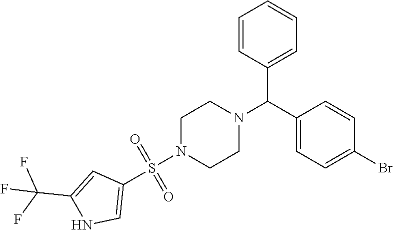 [SMILES: O=S(=O)(c1c[nH]c(C(F)(F)F)c1)N1CCN(C(c2ccccc2)c2ccc(Br)cc2)CC1] | 528.0 | |
| 70 | 4,4′-((4-((5- (Trifluoromethyl)- 1H-pyrrol-3- yl)sulfonyl)piperazin- 1-yl)methylene) dibenzonitrile | 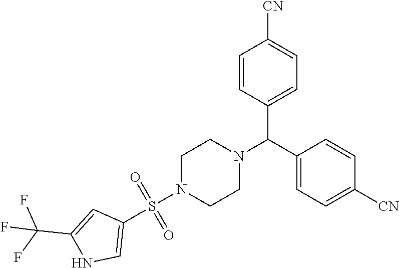 [SMILES: N#Cc1ccc(C(c2ccc(C#N)cc2)N2CCN(S(=O)(=O)c3c[nH]c(C(F)(F)F)c3)CC2)cc1] | 500.1 | |
| 71 | 1-((4- Fluorophenyl)(phenyl) methyl)-4-((5- (trifluoromethyl)-1H- pyrrol-3- yl)sulfonyl)piperazine | 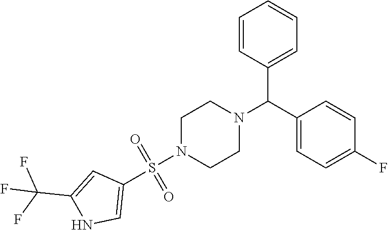 [SMILES: O=S(=O)(c1c[nH]c(C(F)(F)F)c1)N1CCN(C(c2ccccc2)c2ccc(F)cc2)CC1] | 468.1 | ¹H NMR (600 MHz, DMSO-d₆) δ 13.26 (s, 1H), 7.67 (s, 1H), 7.57 − 6.99 (m, 9H), 6.93 (s, 1H), 4.37 (s, 1H), 2.86 (s, 4H), 2.39 (s, partial overlap with solvent peak).^{†} |
| 72 | 1-((2- Bromophenyl)(phenyl) methyl)-4-((5- (trifluoromethyl)-1H- pyrrol-3- yl)sulfonyl)piperazine | 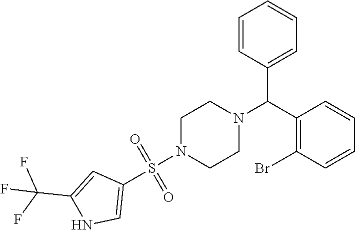 [SMILES: O=S(=O)(c1c[nH]c(C(F)(F)F)c1)N1CCN(C(c2ccccc2)c2ccccc2Br)CC1] | 528.0 | |
| 73 | 1-((4- Chlorophenyl)(4- (trifluoromethyl) phenyl)methyl)-4-((5- (trifluoromethyl)-1H- pyrrol-3- yl)sulfonyl)piperazine | 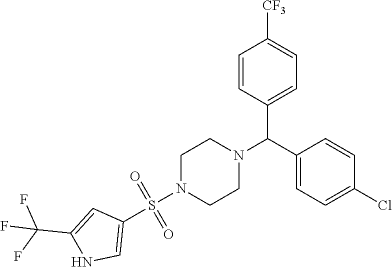 [SMILES: O=S(=O)(c1c[nH]c(C(F)(F)F)c1)N1CCN(C(c2ccc(Cl)cc2)c2ccc(C(F)(F)F)cc2)CC1] | 552.1 | |
| 74 | 1-((2- Fluorophenyl)(phenyl) methyl)-4-((5- (trifluoromethyl)-1H- pyrrol-3- yl)sulfonyl)piperazine | 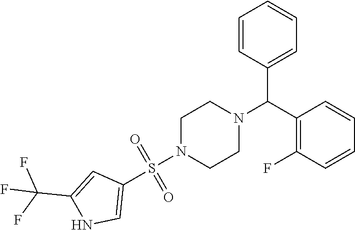 [SMILES: O=S(=O)(c1c[nH]c(C(F)(F)F)c1)N1CCN(C(c2ccccc2)c2ccccc2F)CC1] | 468.1 | ¹H NMR (600 MHz, DMSO-d₆) δ 13.22 (s, 1H), 7.64 − 7.60 (m, 1H), 7.59 − 7.57 (t, 1H), 7.36 − 7.35 (d, J = 7.6 Hz, 2H), 7.30 − 7.28 (t, J = 7.5 Hz, 2H), 7.26 − 7.24 (m, 1H), 7.23 − 7.20 (t, J = 7.5 Hz, 1H), 7.19 − 7.15 (t, J = |
| | | | | 7.5 Hz, 1H), |
| | | | | 7.12 − 7.09 (t, J = |
| | | | | 7.5 Hz, 1H), |
| | | | | 6.91 (s, 1H), 4.65 |
| | | | | (s, 1H), 2.89 (s, |
| | | | | 4H), 2.46 − 2.37 |
| | | | | (m, 4H). |
| 75 | 1-((4- Fluorophenyl)(4- methoxyphenyl) methyl)-4-((5- (trifluoromethyl)-1H- pyrrol-3- yl)sulfonyl)piperazine | 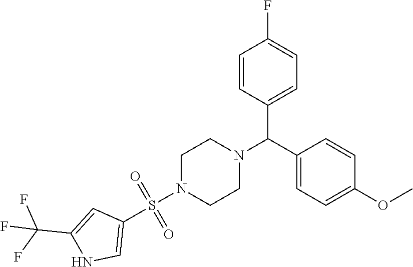 [SMILES: COc1ccc(C(c2ccc(F)cc2)N2CCN(S(=O)(=O)c3c[nH]c(C(F)(F)F)c3)CC2)cc1] | 498.1 | |
| 76 | 1-(Di-p-tolylmethyl)- 4-((5- (trifluoromethyl)-1H- pyrrol-3- yl)sulfony1)piperazine | 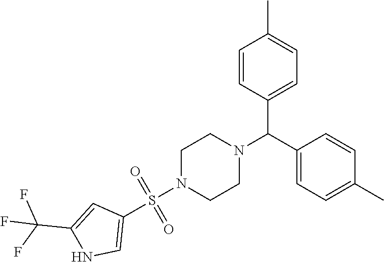 [SMILES: Cc1ccc(C(c2ccc(C)cc2)N2CCN(S(=O)(=O)c3c[nH]c(C(F)(F)F)c3)CC2)cc1] | 478.2 | |
| 77 | 1-([1,1′-Biphenyl]-4- yl(phenyl)methyl)-4- ((5-(trifluoromethyl)- 1H-pyrrol-3- yl)sulfonyl)piperazine | 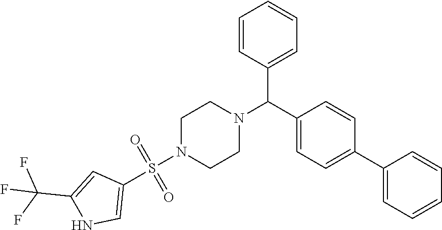 [SMILES: O=S(=O)(c1c[nH]c(C(F)(F)F)c1)N1CCN(C(c2ccccc2)c2ccc(-c3ccccc3)cc2)CC1] | 526.2 | |
| 78 | 1-(Phenyl(o- tolyl)methyl)-4-((5- (trifluoromethyl)-1H- pyrrol-3- yl)sulfonyl)piperazine | 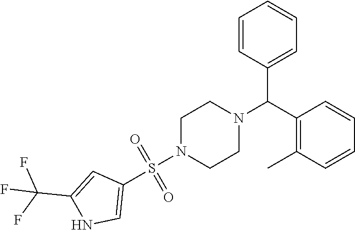 [SMILES: Cc1ccccc1C(c1ccccc1)N1CCN(S(=O)(=O)c2c[nH]c(C(F)(F)F)c2)CC1] | 464.2 | |
| 79 | 1-(Di-o-tolylmethyl)- 4-((5- (trifluoromethyl)-1H- pyrrol-3- yl)sulfonyl)piperazine | 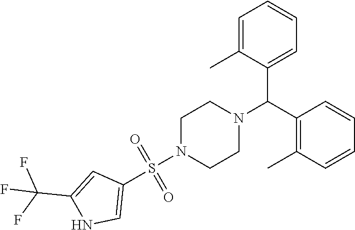 [SMILES: Cc1ccccc1C(c1ccccc1C)N1CCN(S(=O)(=O)c2c[nH]c(C(F)(F)F)c2)CC1] | 478.2 | |
| 80 | 1-(Bis(3- methoxyphenyl) methyl)-4-((5- (trifluoromethyl)-1H- pyrrol-3- yl)sulfonyl)piperazine | 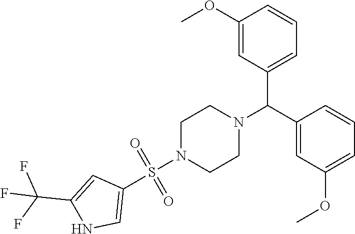 [SMILES: COc1cccc(C(c2cccc(OC)c2)N2CCN(S(=O)(=O)c3c[nH]c(C(F)(F)F)c3)CC2)c1] | 510.2 | |
| 81 | 1-((3- Bromophenyl)(phenyl) methyl)-4-((5- (trifluoromethyl)-1H- pyrrol-3- yl)sulfonyl)piperazine | 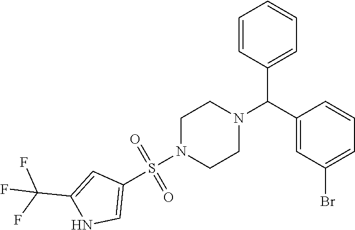 [SMILES: O=S(=O)(c1c[nH]c(C(F)(F)F)c1)N1CCN(C(c2ccccc2)c2cccc(Br)c2)CC1] | 528.0, 530.0 | ¹H NMR (600 MHz, DMSO-d₆) δ 13.23 (s, 1H), 7.66 (s, 1H), 7.62 − 7.54 (m, 1H), 7.50 − 7.12 (m, 8H), 6.92 (s, 1H), 4.37 (s, 1H), 2.90 (br s, 4H), 2.39 (br s, 4H). |
| 82 | 1-((4- (Methylsulfonyl) phenyl)(phenyl) methyl)-4- ((5-(trifluoromethyl)- 1H-pyrrol-3- yl)sulfonyl)piperazine | 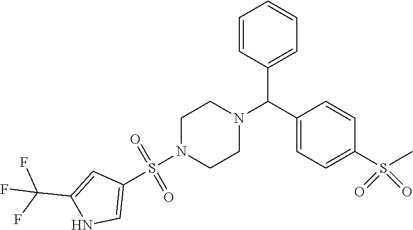 [SMILES: CS(=O)(=O)c1ccc(C(c2ccccc2)N2CCN(S(=O)(=O)c3c[nH]c(C(F)(F)F)c3)CC2)cc1] | 528.1 | ¹H NMR (600 MHz, DMSO-d₆) δ 13.25 (s, 1H), 7.84 (s, 2H), 7.75 − 7.61 (m, 3H), 7.43 (d, J = 7.5 Hz, 2H), 7.30 (d, J = 7.5 Hz, 2H), 7.22 (s, 1H), 6.92 (s, 1H), 4.51 (s, partially resolved peak), 3.16 (s, 3H), 2.89 (s, 4H), 2.42 (s, |
| | | | | 4H).^{†} |
| 83 | 1-(Phenyl(thiophen- 2-yl)methyl)-4-((5- (trifluoromethyl)-1H- pyrrol-3- yl)sulfonyl)piperazine | 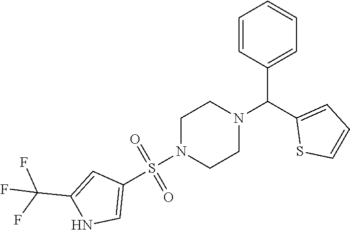 [SMILES: O=S(=O)(c1c[nH]c(C(F)(F)F)c1)N1CCN(C(c2ccccc2)c2cccs2)CC1] | 456.1 | ¹H NMR (600 MHz, DMSO-d₆) δ 13.25 (s, 1H), 7.66 (s, 1H), 7.55 − 7.16 (m, 6H), 6.92 (m, 3H), 4.76 (s, partially resolved peak), 2.84 (br s, 4H), 2.43 (br s, partial overlap with solvent peak).^{†} |
| 84 | 1-(9H-Fluoren-9-yl)- 4-((5- (trifluoromethyl)-1H- pyrrol-3- yl)sulfonyl)piperazine | 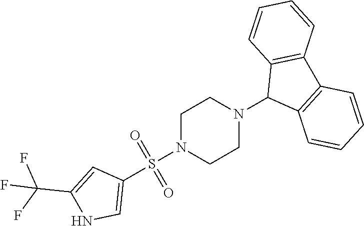 [SMILES: O=S(=O)(c1c[nH]c(C(F)(F)F)c1)N1CCN(C2c3ccccc3-c3ccccc32)CC1] | 448.1 | ¹H NMR (600 MHz, DMSO-d₆) δ 13.22 (s, 1H), 7.96 − 7.80 (m, 2H), 7.64 (d, J = 17.7 Hz, 3H), 7.45 (s, 2H), 7.33 (q, J = 6.3 Hz, 2H), 6.92 (s, 1H), 4.97 (br s, 1H), 2.69 (m, 4H), 2.40 (br s, partial overlap with solvent |
| | | | | peak).^{†} |
| 85 | Methyl 4-(phenyl(4- ((5-(trifluoromethyl)- 1H-pyrrol-3- yl)sulfonyl)piperazin- 1-yl)methyl)benzoate | 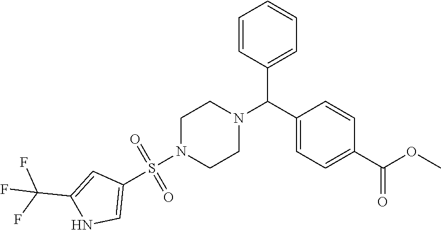 [SMILES: COC(=O)c1ccc(C(c2ccccc2)N2CCN(S(=O)(=O)c3c[nH]c(C(F)(F)F)c3)CC2)cc1] | 508.1 | |
| 86 | 4-(Phenyl(4-((5- (trifluoromethyl)-1H- pyrrol-3- yl)sulfonyl)piperazin- 1-yl)methyl)benzoic acid | 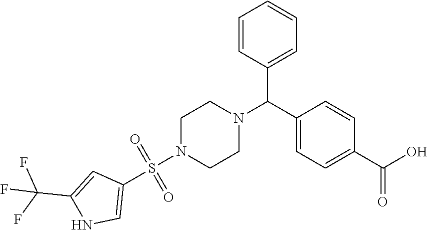 [SMILES: O=C(O)c1ccc(C(c2ccccc2)N2CCN(S(=O)(=O)c3c[nH]c(C(F)(F)F)c3)CC2)cc1] | 494.1 | |
| 87 | 1- (Cyclopentyl(phenyl) methyl)-4-((5- (trifluoromethyl)-1H- pyrrol-3- yl)sulfonyl)piperazine | 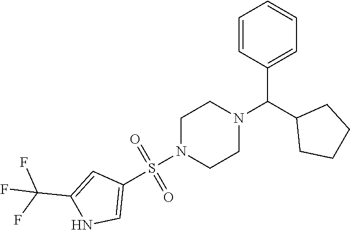 [SMILES: O=S(=O)(c1c[nH]c(C(F)(F)F)c1)N1CCN(C(c2ccccc2)C2CCCC2)CC1] | 442.2 | |
| 88 | 1-(2-Ethyl-1- phenylbutyl)-4-((5- (trifluoromethyl)-1H- pyrrol-3- yl)sulfonyl)piperazine | 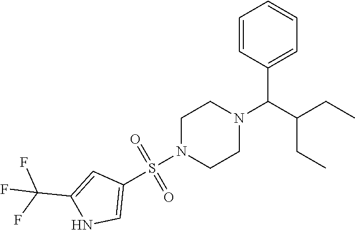 [SMILES: CCC(CC)C(c1ccccc1)N1CCN(S(=O)(=O)c2c[nH]c(C(F)(F)F)c2)CC1] | 444.2 | ¹H NMR (600 MHz, DMSO-d₆) δ 13.16 (s, 1H), 7.59 (s, 1H), 7.34 (t, J = 7.4 Hz, 2H), 7.27 (t, J = 7.3 Hz, 1H), 7.16 (d, J = 7.5 Hz, 2H), 6.86 (s, 1H), 2.81 (s, 4H), 2.45 − 2.12 (m, 4H), 1.97 (m, 1H), 1.46 (m, 1H), 1.35 (m, |
| | | | | 1H), 1.15 (m, |
| | | | | 1H), 0.90-0.85 |
| | | | | (m, 1H), 0.83 (t, |
| | | | | J = 7.4 Hz, 3H), |
| | | | | 0.68 (t, J = 7.4 |
| | | | | Hz, 3H).^{†} |
| 89 | 1-((4- Chlorophenyl) (cyclobutyl) methyl)-4-((5- (trifluoromethyl)-1H- pyrrol-3- yl)sulfonyl)piperazine | 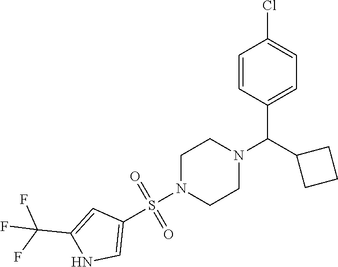 [SMILES: O=S(=O)(c1c[nH]c(C(F)(F)F)c1)N1CCN(C(c2ccc(Cl)cc2)C2CCC2)CC1] | 462.1 | |
| 90 | 1 −((4- Chlorophenyl) (cyclohexyl) methyl)-4-((5- (trifluoromethyl)-1H- pyrrol-3- yl)sulfonyl)piperazine | 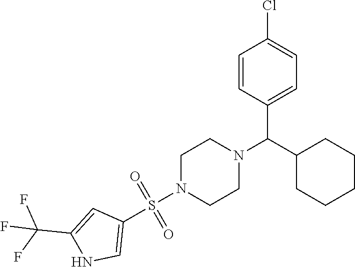 [SMILES: O=S(=O)(c1c[nH]c(C(F)(F)F)c1)N1CCN(C(c2ccc(Cl)cc2)C2CCCCC2)CC1] | 490.1 | |
| 91 | 1-(2-Methyl-1- phenylpropyl)-4-((5- (trifluoromethyl)-1H- pyrrol-3- yl)sulfonyl)piperazine | 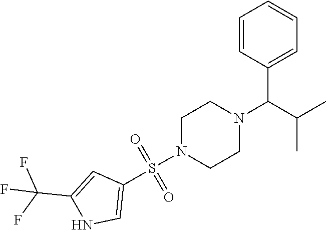 [SMILES: CC(C)C(c1ccccc1)N1CCN(S(=O)(=O)c2c[nH]c(C(F)(F)F)c2)CC1] | 416.2 | |
| 92 | 1-(Cyclopropyl(4- (trifluoromethyl) phenyl)methyl)-4-((5- (trifluoromethyl)-1H- pyrrol-3- yl)sulfonyl)piperazine | 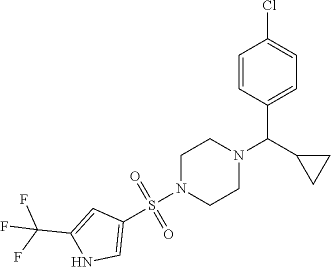 [SMILES: O=S(=O)(c1c[nH]c(C(F)(F)F)c1)N1CCN(C(c2ccc(Cl)cc2)C2CC2)CC1] | 482.1 | |
| 93 | N,2-Diphenyl-2-(4- ((5-(trifluoromethyl)- 1H-pyrrol-3- yl)sulfonyl)piperazin- 1-yl)acetamide | 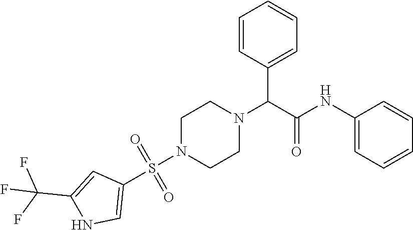 [SMILES: O=C(Nc1ccccc1)C(c1ccccc1)N1CCN(S(=O)(=O)c2c[nH]c(C(F)(F)F)c2)CC1] | 493.1 | |
| 94 | N-Benzyl-2-phenyl-2- (4-((5- (trifluoromethyl)-1H- pyrrol-3- yl)sulfonyl)piperazin- 1-yl)acetamide | 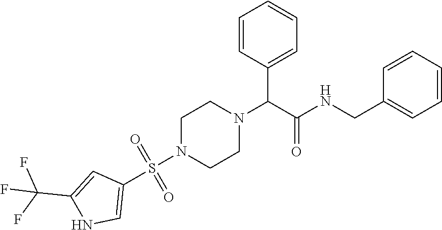 [SMILES: O=C(NCc1ccccc1)C(c1ccccc1)N1CCN(S(=O)(=O)c2c[nH]c(C(F)(F)F)c2)CC1] | 507.2 | |

^{†}Incomplete ¹H NMR chemical shift and integration assignments due to overlap with solvent and/or water peak.

### Example 95 and Example 96. 4-((4-Chlorophenyl)(4-((5-(trifluoromethyl)-1H-pyrrol-3-yl)sulfonyl)piperazin-1-yl)methyl)benzonitrile, Chiral Peak 1 and Chiral Peak 2

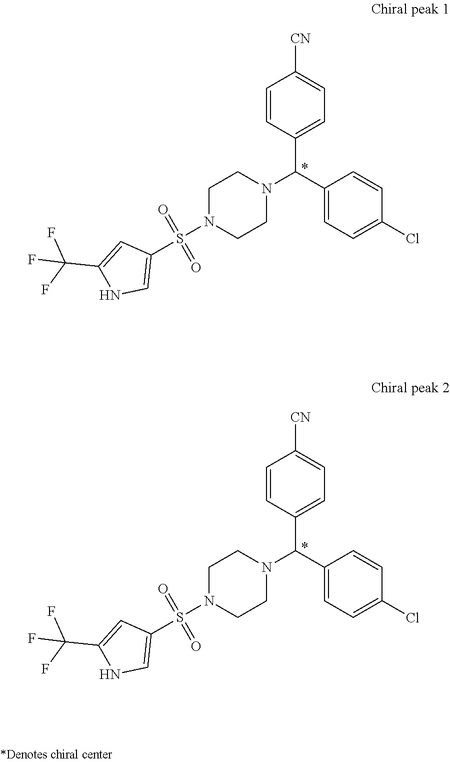 [SMILES: N#Cc1ccc(C(c2ccc(Cl)cc2)N2CCN(S(=O)(=O)c3c[nH]c(C(F)(F)F)c3)CC2)cc1 ; N#Cc1ccc(C(c2ccc(Cl)cc2)N2CCN(S(=O)(=O)c3c[nH]c(C(F)(F)F)c3)CC2)cc1]

6.0 mg of Example 67 was separated by chiral HPLC separation (Phenomenex Lux 5 μm i-Amylose-1. 21.2×250 mm column, eluting with a gradient of 30% EtOH in hexanes, at flow rate of 20 mL/min) to provide chiral peak 1 (Example 95) (1.2 mg, 20%) and chiral peak 2 (Example 96) (2.1 mg, 35%) both as the free base.

Example 95: LCMS calculated for C₂₃H₂₁ClF₃N₄O₂S (M+H)⁺: m/z=509.1. Found: 509.1.

Example 96: LCMS calculated for C₂₃H₂₁ClF₃N₄O₂S (M+H)⁺: m/z=509.1. Found: 509.1.

### Example 97. 4-(Phenyl(4-((5-(trifluoromethyl)-1H-pyrrol-3-yl)sulfonyl)piperazin-1-yl)methyl)benzaldehyde

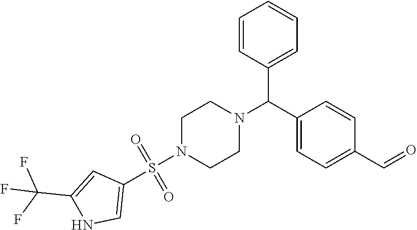 [SMILES: O=Cc1ccc(C(c2ccccc2)N2CCN(S(=O)(=O)c3c[nH]c(C(F)(F)F)c3)CC2)cc1]

To a stirring solution of 4-(phenyl(4-((5-(trifluoromethyl)-1H-pyrrol-3-yl)sulfonyl)piperazin-1-yl)methyl)benzonitrile (Example 63) (20.0 mg, 42 μmol) in THF (0.40 mL) and 4 drops of Et₃N at 0° C. was added lithium aluminum hydride (1M solution in THF, 84 μL, 0.084 mmol). The reaction mixture was stirred at 0° C. for 3 h and then quenched with ice water and NH₄Cl, diluted with methanol, filtered, and purified by preparative reverse-phase HPLC (pH=2) to afford the desired product (7.2 mg, 36%). LCMS calculated for C₂₃H₂₃F₃N₃O₃S (M+H)⁺: m/z=478.1. found 478.2.

### Example 98. (4-(Phenyl(4-((5-(trifluoromethyl)-1H-pyrrol-3-yl)sulfonyl)piperazin-1-yl)methyl)phenyl)methanol

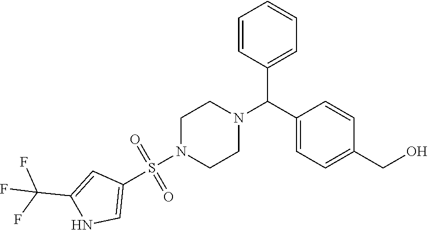 [SMILES: O=S(=O)(c1c[nH]c(C(F)(F)F)c1)N1CCN(C(c2ccccc2)c2ccc(CO)cc2)CC1]

To a stirring solution of 4-(phenyl(4-((5-(trifluoromethyl)-1H-pyrrol-3-yl)sulfonyl)piperazin-1-yl)methyl)benzaldehyde (Example 97) (4 mg, 7.96 μmol) in methanol (0.1 mL), one drop of Et₃N was added followed by sodium borohydride (1.5 mg, 0.04 mmol). The reaction mixture was stirred at rt for 60 min. The reaction was quenched with ice water and NH₄Cl, diluted with methanol, filtered, and purified by preparative reverse-phase HPLC (pH=2) to afford the desired product (2.0 mg, 52%). LCMS calculated for C₂₃H₂₅F₃N₃O₃S (M+H)⁺: m/z=480.2. found 480.2.

### Example 99. 4-(Phenyl(4-((5-(trifluoromethyl)-1H-pyrrol-3-yl)sulfonyl)piperazin-1-yl)methyl)benzamide

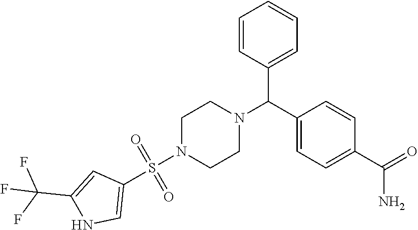 [SMILES: NC(=O)c1ccc(C(c2ccccc2)N2CCN(S(=O)(=O)c3c[nH]c(C(F)(F)F)c3)CC2)cc1]

To a solution of 4-(phenyl(4-((5-(trifluoromethyl)-1H-pyrrol-3-yl)sulfonyl)piperazin-1-yl)methyl)benzonitrile (Example 63) (5.0 mg, 10.54 μmol) in 0.1 mL of concentrated ammonia solution (3.7 mg, 0.11 mmol) at rt was added 0.1 mL of 30% H₂O₂ dropwise, followed by ethanol (0.10 mL). The resulting reaction mixture was stirred at rt for 40 min. The mixture was diluted with water and methanol and purified by preparative reverse-phase HPLC (pH=2) to afford the desired product (2.0 mg, 38%). LCMS calculated for C₂₃H₂₄F₃N₄O₃S (M+H)⁺: m/z=493.1. found 493.1.

### Example 100. 1-((4-(1H-Tetrazol-5-yl)phenyl)(phenyl)methyl)-4-((5-(trifluoromethyl)-1H-pyrrol-3-yl)sulfonyl)piperazine

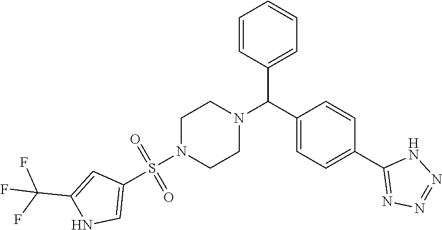 [SMILES: O=S(=O)(c1c[nH]c(C(F)(F)F)c1)N1CCN(C(c2ccccc2)c2ccc(-c3nnn[nH]3)cc2)CC1]

To a mixture of 4-(phenyl(4-((5-(trifluoromethyl)-1H-pyrrol-3-yl)sulfonyl)piperazin-1-yl)methyl)benzonitrile (Example 63) (5.0 mg, 10.5 μmol) in DMF (0.10 mL) was added sodium azide (2.1 mg, 0.03 mmol). The reaction mixture was heated at 130° C. for 3 h, cooled and then diluted with THF, filtered, and dried. The crude product was purified by preparative reverse-phase HPLC (pH=2) to afford the desired product (1.1 mg, 20%). LCMS calculated for C₂₃H₂₃F₃N₇O₂S (M+H)⁺: m/z=518.2. found 518.2.

### Example 101. N-(4-(Phenyl(4-((5-(trifluoromethyl)-1H-pyrrol-3-yl)sulfonyl)piperazin-1-yl)methyl)benzyl)-5-(trifluoromethyl)-1H-pyrrole-3-sulfonamide

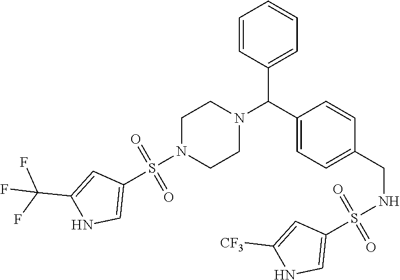 [SMILES: O=S(=O)(NCc1ccc(C(c2ccccc2)N2CCN(S(=O)(=O)c3c[nH]c(C(F)(F)F)c3)CC2)cc1)c1c[nH]c(C(F)(F)F)c1]

### Step 1. (4-(Phenyl(piperazin-1-yl)methyl)phenyl)methanamine

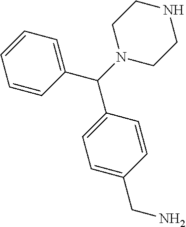 [SMILES: NCc1ccc(C(c2ccccc2)N2CCNCC2)cc1]

To a stirring solution of tert-butyl 4-((4-cyanophenyl)(phenyl)methyl)piperazine-1-carboxylate (12.0 mg, 0.032 mmol) (from Example 63, Step 1) in THF (0.25 mL) at 0° C., two drops of Et₃N were added followed by lithium aluminum hydride (1M solution in THF, 212 μL, added in two portions). The reaction mixture was stirred at 0° C. for 3 h and then quenched with ice water and NH₄Cl. The mixture was diluted with methanol, filtered, and purified by preparative reverse-phase HPLC (pH=2) to afford the product (8.4 mg, 94%). LCMS calculated for C₁₈H₂₄N₃(M+H)⁺: m/z=282.2. found 282.2.

### Step 2. N-(4-(Phenyl(4-((5-(trifluoromethyl)-1H-pyrrol-3-yl)sulfonyl)piperazin-1-yl)methyl)benzyl)-5-(trifluoromethyl)-1H-pyrrole-3-sulfonamide

To a mixture of (4-(phenyl(piperazin-1-yl)methyl)phenyl)methanamine (8.4 mg, 0.03 mmol) in THF (0.3 mL) was added triethylamine (0.017 mL, 0.12 mmol) followed by 5-(trifluoromethyl)-1H-pyrrole-3-sulfonyl chloride (20.9 mg, 0.09 mmol). The resulting mixture was stirred at room temperature for 20 min. The mixture was diluted with water and methanol and purified by preparative reverse-phase HPLC (pH=2) to afford the desired product (5.0 mg, 25%). LCMS calculated for C₂₈H₂₈F₆N₅O₄S₂ (M+H)⁺: m/z=676.1. Found: 676.1.

### Example 102. (4-(Phenyl(4-((5-(trifluoromethyl)-1H-pyrrol-3-yl)sulfonyl)piperazin-1-yl)methyl)phenyl)methanamine

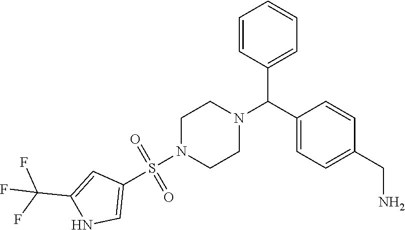 [SMILES: NCc1ccc(C(c2ccccc2)N2CCN(S(=O)(=O)c3c[nH]c(C(F)(F)F)c3)CC2)cc1]

To a stirring solution of 4-(phenyl(4-((5-(trifluoromethyl)-1H-pyrrol-3-yl)sulfonyl)piperazin-1-yl)methyl)benzonitrile (Example 63) (20.0 mg, 0.042 mmol) in THF (0.4 mL) at 0° C. were added 4 drops of Et₃N followed by lithium aluminum hydride (1M solution in THF, 169 μL, 0.169 mmol). The reaction mixture was stirred at 0° C. for 2.5 h. The reaction was quenched with ice water and NH₄Cl, diluted with methanol, filtered, and purified by preparative reverse-phase HPLC (pH=2) to afford the desired product (5.5 mg, 27%). LCMS calculated for C₂₃H₂₆F₃N₄O₂S (M+H)⁺: m/z=479.2. found 479.2.

### Example 103. 1-Methyl-3-(4-(phenyl(4-((5-(trifluoromethyl)-1H-pyrrol-3-yl)sulfonyl)piperazin-1-yl)methyl)benzyl)urea

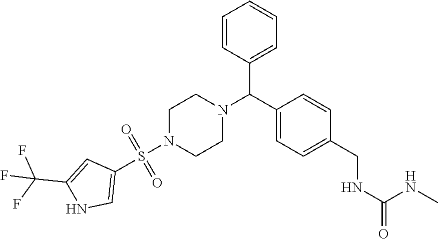 [SMILES: CNC(=O)NCc1ccc(C(c2ccccc2)N2CCN(S(=O)(=O)c3c[nH]c(C(F)(F)F)c3)CC2)cc1]

A solution of (4-(phenyl(4-((5-(trifluoromethyl)-1H-pyrrol-3-yl)sulfonyl)piperazin-1-yl)methyl)phenyl)methanamine (Example 102) (5.0 mg, 10.5 μmol) in THF (0.10 mL) was treated with triethylamine (3.2 mg, 0.03 mmol) followed by the addition of methylcarbamic chloride (1.5 mg, 0.016 mmol). The reaction mixture was stirred at 25° C. overnight and then diluted with methanol and water and purified by preparative reverse-phase HPLC (pH=2) to afford the desired product (2.0 mg, 36%). LCMS calculated for C₂₅H₂₉F₃N₅O₃S (M+H)⁺: m/z=536.2. found 536.2.

### Example 104. N-(4-(Phenyl(4-((5-(trifluoromethyl)-1H-pyrrol-3-yl)sulfonyl)piperazin-1-yl)methyl)benzyl)isonicotinamide

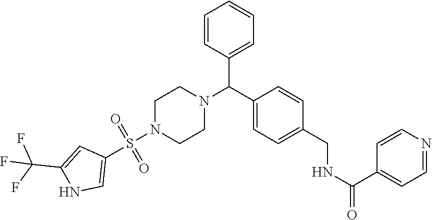 [SMILES: O=C(NCc1ccc(C(c2ccccc2)N2CCN(S(=O)(=O)c3c[nH]c(C(F)(F)F)c3)CC2)cc1)c1ccncc1]

A mixture of (4-(phenyl(4-((5-(trifluoromethyl)-1H-pyrrol-3-yl)sulfonyl)piperazin-1-yl)methyl)phenyl)methanamine (Example 102) (5.0 mg, 10.5 μmol), isonicotinic acid (1.9 mg, 0.016 mmol) in DMF (0.10 mL) was treated with triethylamine (3.1 mg, 0.03 mmol), followed by the addition of O-(7-Azabenzotriazol-1-yl)-N,N,N′,N′-tetramethyluronium hexafluorophosphate (HATU) (5.96 mg, 0.016 mmol). The reaction mixture was stirred at 25° C. for 3 h and then diluted with methanol and purified by preparative reverse-phase HPLC (pH=2) to afford the desired product (2.6 mg, 42%). LCMS calculated for C₂₉H₂₉F₃N₅O₃S (M+H)⁺: m/z=584.2. found 584.2.

### Example 105. N-Methyl-1-(4-(phenyl(4-((5-(trifluoromethyl)-1H-pyrrol-3-yl)sulfonyl)piperazin-1-yl)methyl)phenyl)methanamine

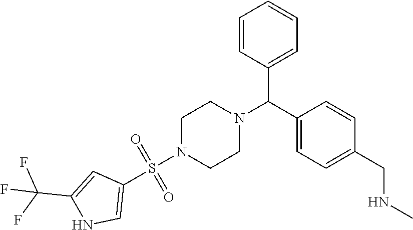 [SMILES: CNCc1ccc(C(c2ccccc2)N2CCN(S(=O)(=O)c3c[nH]c(C(F)(F)F)c3)CC2)cc1]

To a stirring solution of 4-(phenyl(4-((5-(trifluoromethyl)-1H-pyrrol-3-yl)sulfonyl)piperazin-1-yl)methyl)benzaldehyde (Example 97) (5.0 mg, 10.5 μmol) in THF (0.10 mL) was added methylamine (2.0 M solution in THF, 52 μL, 0.11 mmol) and sodium triacetoxyborohydride (6.7 mg, 0.03 mmol). The reaction mixture was stirred at rt for 60 min. The reaction was quenched with ice water, diluted with methanol, filtered, and purified by preparative reverse-phase HPLC (pH=2) to afford the desired product (1.3 mg, 25%). LCMS calculated for C₂₄H₂₈F₃N₄O₂S (M+H)⁺: m/z=493.2. found 493.2.

### Example A. USP18 Enzymatic Assay

The compound's potency of inhibiting USP18 catalyzed de-ISGylation (IC₅₀) was measured as following, using a black 384-well Greiner microtiter plate. The assay reaction consisted of 1 nM His6-GST-3C-hUSP18(aa16-aa372), 100 nM of Ac-ISG15prox-Rh110MP (mouse) (UbiQ Bio, Cat #UbiQ-127) in a buffer consisting of 50 mM Tris pH 7.8, 100 mM NaCl, 0.25 mg/ml Ovalbumin, 1 mg/ml CHAPS, 2 mM TCEP. USP18 enzymatic activity is measured by an increase in fluorescence intensity at ex/em 485/520 nm. In the presence of varying compound concentrations, a reaction mixture is incubated at room temperature for 1 hour and stopped by addition of 5 μL 30 mM citric acid in 20 mM Tris pH 7.8 to final concentration of 10 mM. Fluorescence intensity is measured on a BMG Pherastar plate reader, with FI 485 520 module. A compound's IC₅₀ is calculated using a 4-parameter dose response according to Equation 1, where Y is the measured fluorescence intensity, L is the low signal control (enzyme 100% inhibited) and H is the high signal control (DMSO only), [C] is compound concentration and h is Hill slope.

$$Y = L + ( H - L ) / ( 1 + 10 \hat{} ( ( log {IC}_{50} - log [ C ] ) * h ) ) . \qquad Equation 1$$

Protein Sequence of His6-GST-3C-hUSP18(16-372) is shown below.

(SEQ ID NO: 1)
MHHHHHHSPILGYWKIKGLVQPTRLLLEYLEEKYEEHLYERDEGDKWRN
KKFELGLEFPNLPYYIDGDVKLTQSMAIIRYIADKHNMLGGCPKERAEI
SMLEGAVLDIRYGVSRIAYSKDFETLKVDFLSKLPEMLKMFEDRLCHKT
YLNGDHVTHPDFMLYDALDVVLYMDPMCLDAFPKLVCFKKRIEAIPQID
KYLKSSKYIAWPLQGWQATFGGGDHPPKLEVLFQGPLAESSQSPADLEE
KKEEDSNMKREQPRERPRAWDYPHGLVGLHNIGQTCCLNSLIQVFVMNV
DFTRILKRITVPRGADEQRRSVPFQMLLLLEKMQDSRQKAVRPLELAYC
LQKCNVPLFVQHDAAQLYLKLWNLIKDQITDVHLVERLQALYTIRVKDS
LICVDCAMESSRNSSMLTLPLSLFDVDSKPLKTLEDALHCFFQPRELSS
KSKCFCENCGKKTRGKQVLKLTHLPQTLTIHLMRFSIRNSQTRKICHSL
YFPQSLDFSQILPMKRESCDAEEQSGGQYELFAVIAHVGMADSGHYCVY
IRNAVDGKWFCFNDSNICLVSWEDIQCTYGNPNYHWQETAYLLVYMKME
C

### Example B. USP7 Enzymatic Assay

The compound's potency of inhibiting USP7 catalyzed de-Ubiquitination (IC₅₀) was measured as following, using a black 384-well Greiner microtiter plate. Assays reactions consist of 100 pM human USP7 (BPS, Cat #80395), 50 nM human ubiquitin-rhodamine 110 (R&D Systems, Cat #U-555-MTO) in a buffer consisting of 20 mM Tris pH 7.8, 2 mM CaCl₂, 0.01% Ovalbumin, 0.01% Triton, 1 mM DTT. USP7 enzymatic activity is measured by an increase in fluorescence intensity at ex/em 485/520 nm. In the presence of varying compound concentrations, a reaction mixture is incubated at room temperature for 1 hour and stopped by addition of 5 μL 30 mM citric acid in 20 mM Tris pH 7.8 to final concentration of 10 mM. Fluorescence intensity is measured on a BMG Pherastar plate reader, with FI 485 520 module. A compound's IC₅₀ is calculated using a 4-parameter dose response according to Equation 2, where Y is the measured fluorescence intensity, L is the low signal control (enzyme 100% inhibited) and H is the high signal control (DMSO only), [C] is compound concentration and h is Hill slope.

$$Y = L + ( H - L ) / ( 1 + 10 \hat{} ( ( log {IC}_{50} - log [ C ] ) * h ) ) . \qquad Equation 2$$

TABLE A

| | USP18 | USP7 |
|---|---|---|
| Example No. | IC₅₀ | IC₅₀ |
| 1 | ++ | +++++ |
| 2 | ++++ | +++++ |
| 3 | + | +++++ |
| 4 | + | +++++ |
| 5 | + | +++++ |
| 6 | + | ++++ |
| 7 | ++++ | +++++ |
| 8 | ++ | +++++ |
| 9 | ++ | +++++ |
| 10 | ++ | +++++ |
| 11 | ++ | +++++ |
| 12 | +++ | +++++ |
| 13 | ++ | +++++ |
| 14 | +++ | +++++ |
| 15 | +++ | ++++ |
| 16 | ++++ | +++++ |
| 17 | ++++ | +++++ |
| 18 | +++ | +++++ |
| 19 | ++ | +++++ |
| 20 | ++ | +++++ |
| 21 | +++ | +++++ |
| 22 | NT | +++++ |
| 23 | ++++ | +++++ |
| 24 | NT | +++++ |
| 25 | +++++ | +++++ |
| 26 | ++++ | +++++ |
| 27 | ++++ | ++++ |
| 28 | ++++ | +++++ |
| 29 | +++++ | +++++ |
| 30 | ++ | +++++ |
| 31 | ++ | +++++ |
| 32 | +++ | ++++ |
| 33 | +++ | +++++ |
| 34 | ++++ | ++++ |
| 35 | +++ | +++++ |
| 36 | ++++ | +++++ |
| 37 | ++ | +++++ |
| 38 | ++ | ++++ |
| 39 | + | +++++ |
| 40 | ++ | +++++ |
| 41 | ++++ | +++++ |
| 42 | + | +++++ |
| 43 | ++++ | NT |
| 44 | ++++ | NT |
| 45 | ++++ | +++++ |
| 46 | ++++ | +++++ |
| 47 | +++ | ++ |
| 48 | ++ | +++++ |
| 49 | +++ | +++++ |
| 50 | +++ | +++++ |
| 51 | ++ | +++++ |
| 52 | +++ | +++++ |
| 53 | ++++ | NT |
| 54 | +++ | +++++ |
| 55 | +++ | +++++ |
| 56 | ++ | +++++ |
| 57 | +++ | +++++ |
| 58 | ++++ | +++++ |
| 59 | +++ | +++++ |
| 60 | ++++ | +++++ |
| 61 | NT | +++++ |
| 62 | ++++ | +++++ |
| 63 | + | ++++ |
| 64 | + | ++++ |
| 65 | + | +++++ |
| 66 | + | +++++ |
| 67 | + | +++++ |
| 68 | + | +++++ |
| 69 | + | +++++ |
| 70 | + | +++++ |
| 71 | ++ | +++++ |
| 72 | ++ | +++++ |
| 73 | ++ | +++++ |
| 74 | ++ | +++++ |
| 75 | ++ | +++++ |
| 76 | ++ | +++++ |
| 77 | ++ | +++++ |
| 78 | ++ | +++++ |
| 79 | ++ | +++++ |
| 80 | ++ | +++++ |
| 81 | ++ | +++++ |
| 82 | +++ | +++++ |
| 83 | +++ | +++++ |
| 84 | +++ | +++++ |
| 85 | ++++ | +++++ |
| 86 | +++++ | ++++ |
| 87 | ++ | +++++ |
| 88 | +++ | +++++ |
| 89 | +++ | +++++ |
| 90 | +++ | +++++ |
| 91 | +++ | +++++ |
| 92 | ++++ | +++++ |
| 93 | ++++ | +++++ |
| 94 | ++++ | +++++ |
| 95 | + | +++++ |
| 96 | ++ | +++++ |
| 97 | + | +++ |
| 98 | +++ | ++++ |
| 99 | +++ | ++ |
| 100 | ++++ | ++++ |
| 101 | +++ | ++++ |
| 102 | ++++ | +++++ |
| 103 | ++++ | +++++ |
| 104 | ++++ | ++++ |
| 105 | ++++ | NT |

NT refers to “not tested”
+ refers to IC₅₀ of ≤500 nM
++ refers to IC₅₀ of >500 nM to ≤2500 nM
+++ refers to IC₅₀ of >2500 nM to ≤10000 nM
++++ refers to IC₅₀ of >10000 nM to ≤50000 nM
+++++ refers to IC₅₀ of >50000 nM

### Example C. USP18 Protein Thermal Shift Assay

The compound's direct binding to human USP18 was demonstrated using protein thermal shift assay. The protein thermal shift assay involves measuring the unfolding of a protein with increasing temperature. Unfolding is measured by the increase in fluorescent signal coming from a dye that binds to hydrophobic regions of a protein that become exposed to solvent as the protein unfolds. The temperature at the midpoint of the fluorescent signal increase is defined as the melting temperature, Tm. The Tm of a protein will change upon specific binding to a partner, such as a ligand or a compound. Normally, specific binding of a ligand to the target protein will increase the target protein's thermal stability, which, in turn, leads to an increase in Tm. The Tm increase can correlate with the affinity of the ligand, the higher the affinity, the larger the Tm shift. USP18 Protein thermal shift assay was conducted in MicroAmp Optical 384 well plates (Thermo Fisher Scientific) in a 10 uL reaction consisting of a buffer containing 10 mM HEPES pH 7.4, 500 mM NaCl, 10 uM test compound, 5 uM of human USP18 (aa16-aa372), 0.5× GloMelt™ Dye (Biotium, Fremont, CA, Cat #33021-1), and 0.2% DMSO. Assays were run in triplicate with USP18 in buffer with 0.2% DMSO as reference. Assays were read on an Applied Biosystem real-time PCR instrument (QuantStudio 7 Flex, Thermo Fisher). Protein melt-curves were generated from the fluorescent readings of the GloMelt™ Dye, Ex/Em at 468/507 nm, with temperature rising from 25° C. to 95° C. at a rate of 3° C./min. The Tm was calculated from the melt curve with Protein Thermal Shift software (Thermofisher) using Boltzmann curve fitting method. ΔTm of samples with compounds were calculated based on the reference Tm (Table B).

TABLE B

| Example No. | ΔTm (° C.) |
|---|---|
| 1 | C |
| 9 | C |
| 4 | B |
| 66 | B |
| 67 | A |
| 63 | A |
| 65 | A |

A: ΔTm > 6° C.;
B: ΔTm = 4.5° C. to 6° C.;
C: ΔTm = 2° C. to <4.5° C.

### Example D. Western Blot Analysis of ISG15 and ISG15-Bound to Multiple Interferon Stimulated Gene Protein Products (i.e., ISGylation)

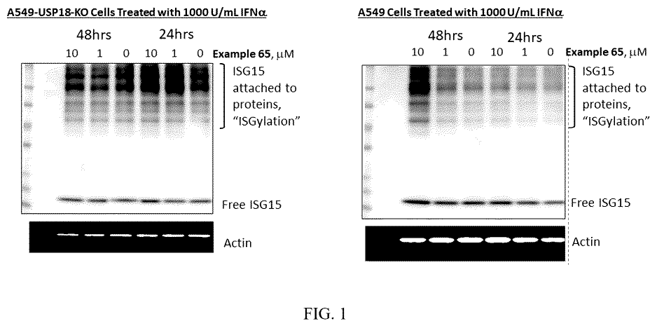

The ability of a selective USP18 inhibitor of the present invention, namely, Example 65 to inhibit the removal of ISG15 from proteins (i.e., de-ISGylation), thereby increasing the observed amount of “ISGylation” was measured using a standard Western Blot analysis. Treatment of A549 (lung adenocarcinoma) cells with 1000 U/mL of IFNα induces the production of ISG15, ISGylated proteins (i.e., ISGylation), and USP18. ISG15 was detected in Western blot using ISG15 recombinant rabbit monoclonal antibody (Thermo Fisher Scientific catalog #MA5-29371), USP18 was detected using rabbit monoclonal antibody USP18 (D47E) (Cell Signaling Technologies Catalog #4813), the secondary antibody used was Anti-rabbit IgG, HRP-linked antibody (Cell Signaling Technologies Catalog #7074). When USP18 is knocked-out of A549 cells (A549-USP18-KO cells), IFNα treatment results in the increased accumulation of ISGylation, compared to wildtype A549 cells treated with IFNα (FIG. 1). Analysis of compound dose response of Example 65 in A549 cells for ISGylated proteins confirm that the compound of the present invention led to increased amounts of ISGylated proteins in A549 cells at 48 hrs (FIG. 1), similar to levels of those observed in A549 cells in which USP18 gene is knocked-out (A549-USP18-KO cells).

### Example E: Western Blot Analysis of Phosphorylated-Signal Transducer and Activator of Transcription 1 (p-STAT1)

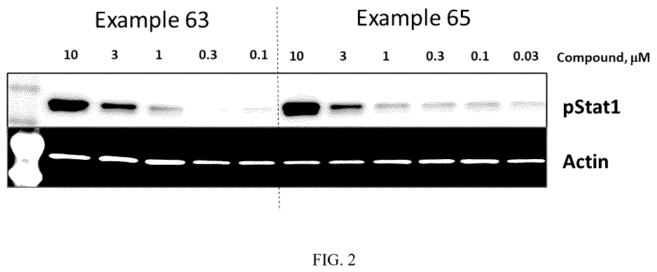

Cells in which USP18 is knocked-out have enhanced activation of interferon alpha/beta receptor (IFNAR) and produce higher levels of phosphorylation of STAT1 and STAT2 upon treatment with IFNα, compared to wildtype cells with intact USP18. (Francois-Newton V, et al. “USP18 establishes the T transcriptional and anti-proliferative interferon alpha/beta differential”. Biochem J 2012; 446: 509-516). Thus, the binding of USP18 to IFNAR inhibits IFNAR activity. Additionally, the isoleucine to asparagine mutation at residue 61 in human USP18 impairs its binding to IFNAR and results in increases in phosphorylated STAT1 and phosphorylated STAT2 compared cells with nonmutated USP18 that are treated with IFNα (Martin-Fernandez M, et al. “A partial form of inherited human USP18 deficiency underlies infection and inflammation”. J Exp Med. 2022 219(4): e20211273). The ability of selective USP18 inhibitors of the present invention, namely, Example 63 and Example 65 to prevent USP18 binding to and inhibiting IFNAR can be assessed by observing an increase in the amount of pSTAT1 as measured using a standard Western Blot analysis. Phosphorylated STAT1 was detected in Western blot analysis using Phospho-Stat1 (Tyr701) (58D6) Rabbit mAb (Cell Signaling Technology catalog #9167) and the secondary antibody was Anti-rabbit IgG, HRP-linked antibody (Cell Signaling Technologies Catalog #7074). The assay used A549 cells in which USP18 was knocked-out and then reintroduced to be constitutively overexpressed (A549-USP18-KO/OE). A549-USP18-KO/OE cells were incubated with test compound in dose-response format for 1 hour prior to treatment with 1000 Units/mL IFNα, then incubated for an additional 30 minutes after which the amount of phosphorylated STAT1 (pSTAT1) was assessed by Western Blot. Analysis of compound dose responses for Example 63 and Example 65 confirm that compounds of the present invention led to increased amounts of pSTAT1 protein in response to IFNα treatment (FIG. 2).

### Example F: Homogenous Time Resolved Fluorescence (HTRF) Analysis of pSTAT Activation by Interferon Alpha (IFNα)

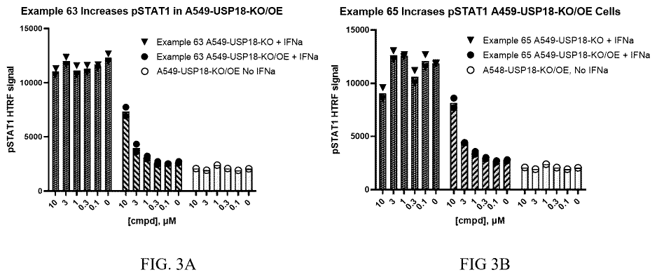

The ability of selective USP18 inhibitors Example 63 and Example 65 to prevent USP18 binding to IFNAR can also be assessed quantitatively using an HTRF assay (Revvity catalog #63ADK026PEG) measuring levels of pSTAT. The HTRF assay used A549 cells in which USP18 was knocked-out (A549-USP18-KO) and A549 cells in which USP18 was knocked-out then reintroduced to be constitutively overexpressed (A549-USP18-KO/OE). A549-USP18-KO and A549-USP18-KO/OE cells were incubated with test compounds for 1 hour prior to incubation with 1000U/mL IFNα for 30 minutes. Analysis of compound Example 63 and Example 65 confirm inhibition of USP18 activity as the levels of pSTAT1 were increased in a dose-dependent manner only in A549-USP18-KO/OE, whereas A549-USP18-KO had consistently high production of pSTAT1 regardless of compound addition. A549-USP18-KO/OE was incubated in absence of IFN as a control to demonstrate dependance of IFNα stimulation which resulted in no pSTAT1 (FIGS. 3A-3B).

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 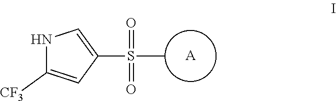
or a pharmaceutically acceptable salt thereof, wherein:
Ring A is independently selected from: 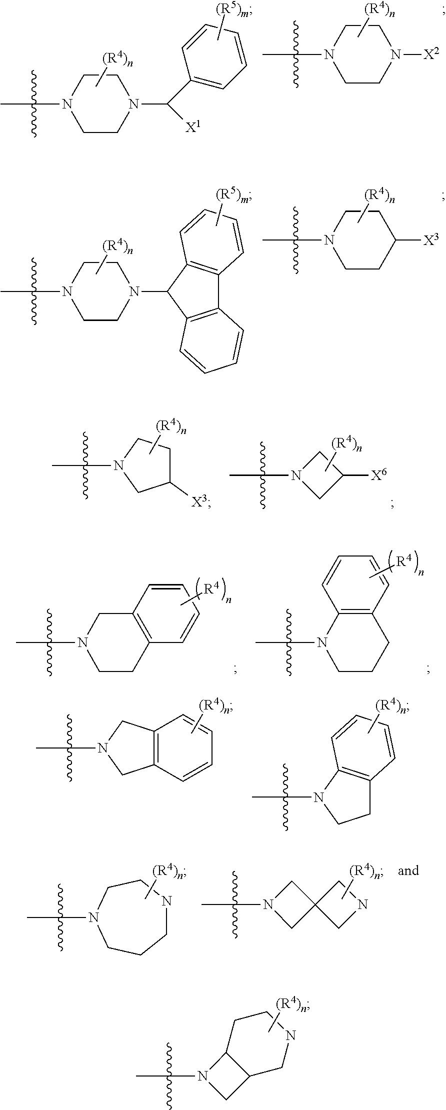
X¹ is selected from C₁₋₆ alkyl, C₆₋₁₀ aryl, C₃₋₁₀ cycloalkyl, 5-10 membered heteroaryl, 4-10 membered heterocycloalkyl, C(O)NHR^{a1}, and C(O)OR^{a1}, wherein the C₁₋₆ alkyl, C₆₋₁₀ aryl, C₃₋₁₀ cycloalkyl, 5-10 membered heteroaryl, and 4-10 membered heterocycloalkyl of X¹ are each optionally substituted with 1, 2, 3, 4, 5, 6, 7, or 8 independently selected R¹ substituents;
each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NRCC(═NR^{c1})R^{b1}, NR^{c1}S(O)R^{b1}, NR^{c1}S(O)NR^{c1}R^{d1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, and OS(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹ are each optionally substituted with 1, 2, 3, 4, 5, 6, 7, or 8 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a1}, R^{c1} and R^{d1} are each optionally substituted with 1, 2, 3, 4, 5, 6, 7, or 8 independently selected R^{1A} substituents;
or, any R^{c1} and R^{d1} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1A} substituents;
each R^{b1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b1} are each optionally substituted with 1, 2, 3, 4, 5, 6, 7, or 8 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, 4, 5, 6, 7, or 8 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a11}, R^{c11} and R^{d11} are each optionally substituted with 1, 2, 3, 4, 5, 6, 7, or 8 independently selected R^{1B} substituents;
or, any R^{c11} and R^{d11} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{1B} substituents;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R are each optionally substituted with 1, 2, 3, 4, 5, 6, 7, or 8 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{1B} is independently selected from halo, oxo, C₁₋₆ alkyl, C₁₋₆ haloalkyl, C₂₄ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)R^{b12}, NR^{c12}S(O)NR^{c12}R^{d12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, and OS(O)₂R^{b12}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{1B} are each optionally substituted with 1, 2, 3, or 4 independently selected R^{M} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇cycloalkyl-C₁₋₆ alkyl-, (5-6 membered heteroaryl)-C₁₋₆ alkyl-, and (4-7 membered heterocycloalkyl)-C₁₋₆ alkyl, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl of R^{a12}, R^{c12} and R^{d12} are each optionally substituted with 1, 2, 3, or 4 independently selected R^{M} substituents;
or, any R^{c12} and R^{d12} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b12} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b12} are each optionally substituted with 1, 2, 3, or 4 independently selected R^{M} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl;
X² is selected from C₆₋₁₀ aryl and 5-10 membered heteroaryl, wherein the C₆₋₁₀ aryl and 5-10 membered heteroaryl of X² are each optionally substituted with 1, 2, 3, 4, 5, 6, 7, or 8 independently selected R² substituents;
each R² is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, and OS(O)₂R^{b2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl and 4-10 membered heterocycloalkyl group are each optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21} NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{2A} are each optionally substituted with 1, 2, 3, or 4 independently selected R^{M} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a21}, R^{c21} and R^{d21} are each optionally substituted with 1, 2, 3, or 4 independently selected R^{M} substituents;
or, any R^{c21} and R^{d21} attached to the same N atom, together with the N atom to which they are attached, form a 5-6 membered heteroaryl or a 4-7 membered heterocycloalkyl group, wherein the 5-6 membered heteroaryl and 4-7 membered heterocycloalkyl group are each optionally substituted with 1, 2, 3, or 4 independently selected R^{M} substituents;
each R^{b21} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b21} are each optionally substituted with 1, 2, 3, or 4 independently selected R^{M} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
X³ is selected from C₆₋₁₀ aryl-C₁₋₆ alkyl- and C₆₋₁₀ aryl, wherein the C₆₋₁₀ aryl-C₁₋₆ alkyl- and C₆₋₁₀ aryl of X³ are each optionally substituted with 1, 2, 3, 4, 5, 6, 7, or 8 independently selected R³ substituents;
each R³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)NR^{c3}R^{d3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, and OS(O)₂R^{b3}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, 4, 5, 6, 7, or 8 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a3}, R^{c3} and R^{d3} are each optionally substituted with 1, 2, 3, 4, 5, 6, 7, or 8 independently selected R^{A} substituents;
or, any R^{c3} and R^{d3} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3A} substituents;
each R^{b3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b3} are each optionally substituted with 1, 2, 3, 4, 5, 6, 7, or 8 independently selected R^{3A} substituents;
each R^{e3} is independently selected from H, OH, CN, C₁₋₆ alkyl, C₁ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{3A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, CN, NO₂, OR^{a31}, SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}C(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31}, NR^{c31}S(O)R^{b31}, NR^{c31}S(O)NR^{c31}R^{d31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)═NR^{e3})R^{b31}, and OS(O)₂R^{b31}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{3A} are each optionally substituted with 1, 2, 3, or 4 independently selected R^{M} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₆ alkyl- of R^{a31}, R^{c31} and R^{d31} are each optionally substituted with 1, 2, 3, or 4 independently selected R^{M} substituents;
or, any R^{c31} and R^{d31} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b31} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b31} are each optionally substituted with 1, 2, 3, or 4 independently selected R^{M} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
each R⁴ is independently selected from H, oxo, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, and OS(O)₂R^{b4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁴ are each optionally substituted with 1, 2, 3, 4, 5, 6, 7, or 8 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a4}, R^{c4} and R^{d4} are each optionally substituted with 1, 2, 3, 4, 5, 6, 7, or 8 independently selected R^{4A} substituents;
or, any R^{c4} and R^{d4} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4A} substituents;
each R^{b4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b4} are each optionally substituted with 1, 2, 3, 4, 5, 6, 7, or 8 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{4A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c1}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, and OS(O)₂R^{b41}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{4A} are each optionally substituted with 1, 2, 3, or 4 independently selected R^{M} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a41}, R^{c41} and R^{d41} are each optionally substituted with 1, 2, 3, or 4 independently selected R^{M} substituents;
or, any R^{c41} and R^{d41} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b41} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{b41} are each optionally substituted with 1, 2, 3, or 4 independently selected R^{M} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-,
or, alternatively, two R⁴ groups together form a 7-9 membered bridged bicycle with the ring to which the two R⁴ groups are attached;
each R⁵ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, and OS(O)₂R^{b5}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R are each optionally substituted with 1, 2, 3, 4, 5, 6, 7, or 8 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a5}, R^{c5} and R^{d5} are each optionally substituted with 1, 2, 3, 4, 5, 6, 7, or 8 independently selected R^{5A} substituents;
or, any R^{c5} and R^{d5} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{5A} substituents;
each R^{b5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of RV are each optionally substituted with 1, 2, 3, 4, 5, 6, 7, or 8 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{5A} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{e51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)R^{b51}, NR^{c51}S(O)NR^{c51}R^{d51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, and OS(O)₂R^{b51}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{5A} are each optionally substituted with 1, 2, 3, or 4 independently selected R^{M} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a51}, R^{c51} and R^{d51} are each optionally substituted with 1, 2, 3, or 4 independently selected R^{M} substituents;
or, any R^{c51} and R^{d51} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b51} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{b51} are each optionally substituted with 1, 2, 3, or 4 independently selected R^{M} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-,
X⁶ is selected from C₆₋₁₀ aryl-C₁₋₆ alkyl-, C₆₋₁₀ aryl and 5-10 membered heteroaryl, wherein the C₆₋₁₀ aryl-C₁₋₆ alkyl-, C₆₋₁₀ aryl and 5-10 membered heteroaryl of X⁶ are each optionally substituted with 1, 2, 3, 4, 5, 6, 7, or 8 independently selected R⁶ substituents;
each R⁶ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NOH)R^{b6}, C(═NCN)R^{b6}, C(═NR^{e6})NR^{c6}R^{d6}, NR^{c6}C(═NR^{e6})NR^{c6}R^{d6}, NR^{c6}C(═NOH)NR^{c6}R^{d6}, NR^{c6}C(═NCN)NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, S(O)(═NR^{e6})R^{b6}, OS(O)(═NR^{e6})R^{b6}, and OS(O)₂R^{b6}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁶ are each optionally substituted with 1, 2, 3, 4, 5, 6, 7, or 8 independently selected R^{6A} substituents;
each R^{a6}, R^{b6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a6}, R^{c6} and R^{d6} are each optionally substituted with 1, 2, 3, 4, 5, 6, 7, or 8 independently selected R^{6A} substituents;
or, any R^{c6} and R^{d6}, attached to the same N atom, together with the N atom to which they are attached, form a 5-10 membered heteroaryl group or a 4-10 membered heterocycloalkyl group, wherein the 5-10 membered heteroaryl group or 4-10 membered heterocycloalkyl group is optionally substituted with 1, 2, 3, 4, 5, 6, 7, or 8 independently selected R^{6A} substituents;
each R^{b6} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b6} are each optionally substituted with 1, 2, 3, 4, 5, 6, 7, or 8 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{6A} is selected from H, halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NOH)R^{b61}, C(═NCN)R^{b61}, C(═NR^{e61})NR^{c61}R^{d61}, NR^{c61}C(═NR^{e61})NR^{c61}R^{d61}, NR^{c61}C(═NOH)NR^{c61}R^{d61}, NR^{c61}C(═NCN)NR^{c61}R^{d61}, NR^{c61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, S(O)(═NR^{e61})R^{b61}, OS(O)(═NR^{e61})R^{b61}, and OS(O)₂R^{b61}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{6A} are each optionally substituted with 1, 2, 3, or 4 independently selected R^{M} substituents;
each R^{a61}, R^{c61}, and R^{d61} is independently selected from H, C₁₋₆ alkyl, Cia-haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{a61}, R^{c61} and R^{d61} are each optionally substituted with 1, 2, 3, or 4 independently selected R^{M} substituents;
or, any R^{c61} and R^{d61}, attached to the same N atom, together with the N atom to which they are attached, form a 5-6 membered heteroaryl group or a 4-7 membered heterocycloalkyl group, wherein the 5-6 membered heteroaryl group or 4-7 membered heterocycloalkyl group is optionally substituted with 1, 2, 3, or 4 independently selected R^{M} substituents;
each R^{b6} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{b6} are each optionally substituted with 1, 2, 3, or 4 independently selected R^{M} substituents;
each R^{e6} is independently selected from H, OH, CN, C₁₋₆ alkyl, C₁ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-;
n is 0, 1, or 2;
m is 0, 1, or 2; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-; and
provided that X² is not 2-chlorophenyl.

2. The compound of claim 1, or a pharmaceutically acceptable salt thereof, wherein Ring A is

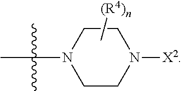

3. The compound of claim 1, or a pharmaceutically acceptable salt thereof, wherein X² is selected from C₆₋₁₀ aryl and 5-10 membered heteroaryl, wherein the C₆₋₁₀ aryl and 5-10 membered heteroaryl of X² are each optionally substituted with 1, 2, 3, or 4 independently selected R² substituents.

4. (canceled)

5. The compound of claim 1, or a pharmaceutically acceptable salt thereof, wherein each R² is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, and NR^{c2}R^{d2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{2A} is independently selected from halo, C₁₋₆ alkyl, C₁₋₆ haloalkyl, C₂₋₆ alkenyl, and C₂₋₆ alkynyl.

6.-12. (canceled)

13. The compounds of claim 1, or a pharmaceutically acceptable salt thereof, wherein each R⁴ is independently selected from H, halo, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R⁴ are each optionally substituted with 1, 2, or 3 independently selected R^{4A} substituents,
or, alternatively, two R⁴ groups together form a 7-9 membered bridged bicycle with the ring to which the two R⁴ groups are attached; and
each R^{4A} is hydroxy.

14.-15. (canceled)

16. The compound of claim 1, or a pharmaceutically acceptable salt thereof, wherein Ring A is

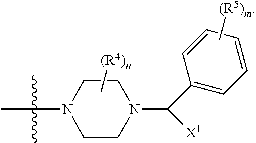

17. The compound of claim 1, or a pharmaceutically acceptable salt thereof, wherein each R⁵ is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)NR^{c5}R^{d5}, NR^{c5}S(O)₂R^{b5}, and S(O)₂R^{b5}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R⁵ are each optionally substituted with 1, 2, 3, or 4 independently selected R^{5A} substituents;
each R^{a5}, R^{b5}, R^{c5}, and R^{d5} is independently selected from H, C₁₋₆ alkyl, C₁₋₆ haloalkyl, C₂₋₆ alkenyl, and C₂₋₆ alkynyl;
each R^{5A} is independently selected from CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R⁵¹, NR^{c51}NR^{c51}R^{d51}, NR^{c51}C(O)R^{b51}, NR^{c51}C(O)OR^{a51}, NR^{c51}C(O)NR^{c51}R^{d51}, CNR^{c51}S(O)R^{b51}, NR^{c51}S(O)NR^{c51}R^{d51}, and NR^{c51}S(O)₂R^{b51};
each R^{a51}, R^{c51}, and R^{d51} is independently selected from H, C₁₋₆ alkyl, C₁₋₆ haloalkyl, C₂₋₆ alkenyl, and C₂₋₆ alkynyl;
each R^{b51} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R^{b51} are each optionally substituted with 1 or 2 independently selected R^{M} substituents; and
each R^{M} is C₁₋₆ alkyl, C₁₋₆haloalkyl, C₂₋₆ alkenyl, and C₂₋₆ alkynyl.

18.-21. (canceled)

22. The compound of claim 1, or a pharmaceutically acceptable salt thereof, wherein X¹ is selected from C₁₋₆ alkyl, C₆₋₁₀ aryl, C₃₋₁₀ cycloalkyl, 5-10 membered heteroaryl, 4-10 membered heterocycloalkyl, C(O)NHR^{a1}, and C(O)OR^{a1}, wherein the C₁₋₆ alkyl, C₆₋₁₀ aryl, C₃₋₁₀ cycloalkyl, 5-10 membered heteroaryl, and 4-10 membered heterocycloalkyl of X¹ are each optionally substituted with 1, 2, 3, or 4 independently selected R¹ substituents; and
each R^{a1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

23.-24. (canceled)

25. The compound of claim 1, or a pharmaceutically acceptable salt thereof, wherein each R¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, CN, NO₂, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)NR^{c1}R^{d1}, NR^{c1}S(O)₂R^{b1}, and S(O)₂R^{b1}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R¹ are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{b1} is independently selected from H, C₁₋₆ alkyl, C₁₋₆ haloalkyl, C₂₋₆ alkenyl, and C₂₋₆ alkynyl;
each R^{1A} is independently selected from CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, CNR^{c11}S(O)R^{b11}, NR^{c11}(O)NR^{c11}R^{d11}, and NR^{c11}S(O)₂R^{b11};
each R^{a11}, R^{c11}, and R^{d11} is independently selected from H, C₁₋₆ alkyl, C₁₋₆ haloalkyl, C₂₋₆ alkenyl, and C₂₋₆ alkynyl;
each R^{b11} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R^{b11} are each optionally substituted with 1 or 2 independently selected R^{1B} substituents; and
each R^{1B} is independently selected from C₁₋₆ alkyl, C₁₋₆ haloalkyl, C₂₋₆ alkenyl, and C₂₋₆ alkynyl.

26.-30. (canceled)

31. The compound of claim 1, or a pharmaceutically acceptable salt thereof, wherein Ring A is selected from:

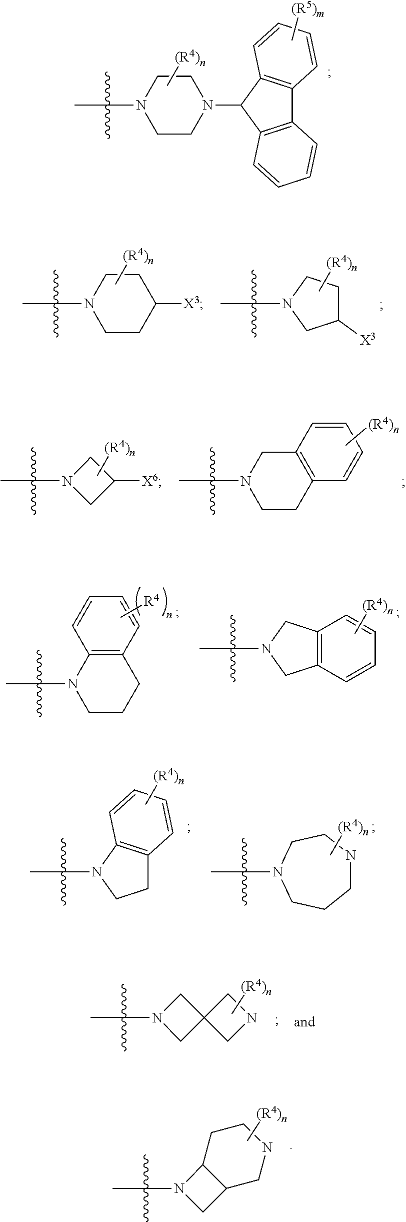

32. The compound of claim 1, or a pharmaceutically acceptable salt thereof, wherein each R⁴ is independently selected from H, halo, C₆₋₁₀ aryl, C₃₋₁₀ cycloalkyl, 5-10 membered heteroaryl, 4-10 membered heterocycloalkyl, and NR^{c4}C(O)OR^{a4}, wherein the C₆₋₁₀ aryl, C₃₋₁₀ cycloalkyl, 5-10 membered heteroaryl, and 4-10 membered heterocycloalkyl of R⁴ are each optionally substituted with 1 or 2 independently selected R^{4A} substituents;
each R^{a4} and R^{c4} is independently selected from H and C₁₋₆ alkyl;
each R^{4A} is independently selected from halo, C₁₋₆ alkyl, C₁₋₆ haloalkyl, C₂₋₆ alkenyl, C₂₋₆ alkynyl, CN, NO₂, C(O)NR^{c41}R^{d41}, C(O)OR^{a41}, and NR^{c41}R^{d41}; and
each R^{a41}, R^{c41} and R^{d41} is independently selected from H, C₁₋₆ alkyl, C₁₋₆ haloalkyl, C₂₋₆ alkenyl, C₂₋₆ alkynyl.

33.-38. (canceled)

39. The compound of claim 1, or a pharmaceutically acceptable salt thereof, wherein X³ is selected from C₆₋₁₀ aryl and C₆₋₁₀ aryl-C₁₋₆ alkyl-, wherein the C₆₋₁₀ aryl and C₆₋₁₀ aryl-C₁₋₆alkyl- of X³ are each optionally substituted with 1 or 2 independently selected R³ substituents.

40. (canceled)

41. The compound of claim 1, or a pharmaceutically acceptable salt thereof, wherein each R³ is independently selected from halo, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, and 4-10 membered heterocycloalkyl.

42.-43. (canceled)

44. The compound of claim 1, or a pharmaceutically acceptable salt thereof, wherein X⁶ is C₆₋₁₀ aryl-C₁₋₆ alkyl-, wherein the C₆₋₁₀ aryl-C₁₋₆ alkyl- is optionally substituted with 1 or 2 independently selected R⁶ substituents; and
each R⁶ is C₆₋₁₀ aryl.

45.-48. (canceled)

49. The compound of claim 1, or a pharmaceutically acceptable salt thereof, wherein the compound of Formula I is a compound of Formula II: 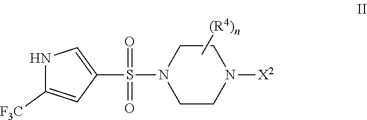
or a pharmaceutically acceptable salt thereof, wherein n is 0, 1, or 2.

50. The compound of claim 1, or a pharmaceutically acceptable salt thereof, wherein the compound of Formula I is a compound of Formula III: 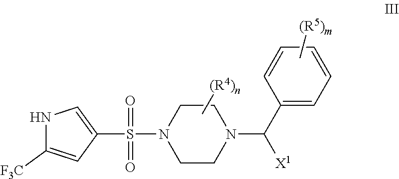
or a pharmaceutically acceptable salt thereof, wherein:
m is 0, 1, or 2; and
n is 0, 1, or 2.

51. The compound of claim 1, or a pharmaceutically acceptable salt thereof, wherein the compound of Formula I is a compound of Formula IV, Formula V, Formula VI, Formula VII, Formula VIII, Formula IX, Formula X, Formula XI, Formula XII, Formula XIII, or Formula XIV: 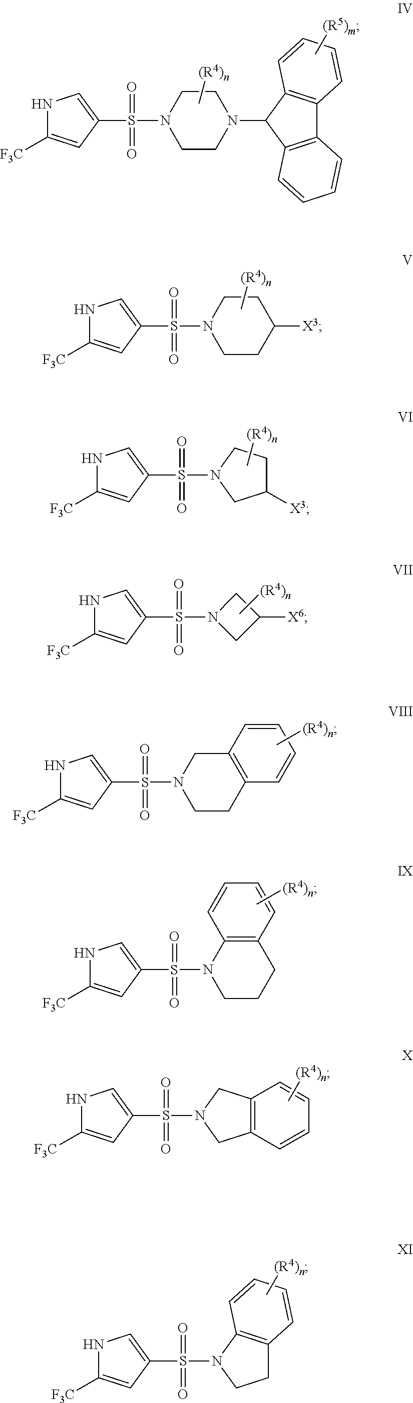 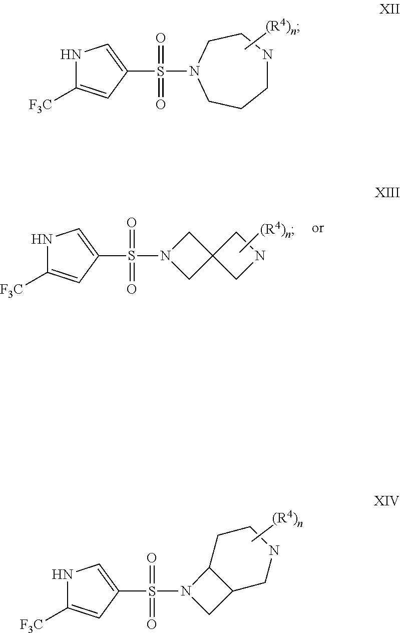
or a pharmaceutically acceptable salt thereof, wherein m is 0, 1, or 2 and n is 0, 1, or 2.

52. The compound of claim 1, selected from:
1-(5-chloro-2-methylphenyl)-4-((5-(trifluoromethyl)-1H-pyrrol-3-yl)sulfonyl)piperazine;
1-(3-nitrophenyl)-4-((5-(trifluoromethyl)-1H-pyrrol-3-yl)sulfonyl)piperazine;
1-(2-chloro-6-methylphenyl)-4-((5-(trifluoromethyl)-1H-pyrrol-3-yl)sulfonyl)piperazine;
1-(2,4-dichlorophenyl)-4-((5-(trifluoromethyl)-1H-pyrrol-3-yl)sulfonyl)piperazine;
1-(2,6-dichlorophenyl)-4-((5-(trifluoromethyl)-1H-pyrrol-3-yl)sulfonyl)piperazine;
1-(4-chloro-2-methylphenyl)-4-((5-(trifluoromethyl)-1H-pyrrol-3-yl)sulfonyl)piperazine;
1-phenyl-4-((5-(trifluoromethyl)-1H-pyrrol-3-yl)sulfonyl)piperazine;
1-(2-ethylphenyl)-4-((5-(trifluoromethyl)-1H-pyrrol-3-yl)sulfonyl)piperazine;
1-(4-fluoro-2-methylphenyl)-4-((5-(trifluoromethyl)-1H-pyrrol-3-yl)sulfonyl)piperazine;
1-((5-(trifluoromethyl)-1H-pyrrol-3-yl)sulfonyl)-4-(2-(trifluoromethyl)phenyl)piperazine;
1-(3-chloro-2-methylphenyl)-4-((5-(trifluoromethyl)-1H-pyrrol-3-yl)sulfonyl)piperazine;
1-(3-methyl-[1,1′-biphenyl]-4-yl)-4-((5-(trifluoromethyl)-1H-pyrrol-3-yl)sulfonyl)piperazine;
1-([1,1′-biphenyl]-2-yl)-4-((5-(trifluoromethyl)-1H-pyrrol-3-yl)sulfonyl)piperazine;
2-(4-((5-(trifluoromethyl)-1H-pyrrol-3-yl)sulfonyl)piperazin-1-yl)benzonitrile;
1-(2-methoxyphenyl)-4-((5-(trifluoromethyl)-1H-pyrrol-3-yl)sulfonyl)piperazine;
2-(4-((5-(trifluoromethyl)-1H-pyrrol-3-yl)sulfonyl)piperazin-1-yl)aniline;
1-(naphthalen-1-yl)-4-((5-(trifluoromethyl)-1H-pyrrol-3-yl)sulfonyl)piperazine;
methyl 4-methyl-3-(4-((5-(trifluoromethyl)-1H-pyrrol-3-yl)sulfonyl)piperazin-1-yl)benzoate;
1-(3,5-dichloropyridin-2-yl)-4-((5-(trifluoromethyl)-1H-pyrrol-3-yl)sulfonyl)piperazine;
1-(3,5-dichloropyridin-4-yl)-4-((5-(trifluoromethyl)-1H-pyrrol-3-yl)sulfonyl)piperazine;
1-(3-chloro-5-(trifluoromethyl)pyridin-2-yl)-4-((5-(trifluoromethyl)-1H-pyrrol-3-yl)sulfonyl)piperazine;
6-bromo-1-((5-(trifluoromethyl)-1H-pyrrol-3-yl)sulfonyl)-1,2,3,4-tetrahydroquinoline;
7-bromo-1-((5-(trifluoromethyl)-1H-pyrrol-3-yl)sulfonyl)-1,2,3,4-tetrahydroquinoline;
5-bromo-1-((5-(trifluoromethyl)-1H-pyrrol-3-yl)sulfonyl)-1,2,3,4-tetrahydroquinoline;
1-((5-(trifluoromethyl)-1H-pyrrol-3-yl)sulfonyl)-1,2,3,4-tetrahydroquinoline;
7-phenyl-2-((5-(trifluoromethyl)-1H-pyrrol-3-yl)sulfonyl)-1,2,3,4-tetrahydroisoquinoline;
5-fluoro-2-((5-(trifluoromethyl)-1H-pyrrol-3-yl)sulfonyl)isoindoline;
7-bromo-1-((5-(trifluoromethyl)-1H-pyrrol-3-yl)sulfonyl)indoline;
1-((5-(trifluoromethyl)-1H-pyrrol-3-yl)sulfonyl)indoline;
4-(2-chlorophenyl)-1-((5-(trifluoromethyl)-1H-pyrrol-3-yl)sulfonyl)piperidine;
4-(5-chloro-2-methylphenyl)-1-((5-(trifluoromethyl)-1H-pyrrol-3-yl)sulfonyl)piperidine;
4-benzhydryl-1-((5-(trifluoromethyl)-1H-pyrrol-3-yl)sulfonyl)piperidine;
4-((3-(2-chlorophenyl)pyrrolidin-1-yl)sulfonyl)-2-(trifluoromethyl)-1H-pyrrole;
trans-tert-butyl (4-(4-fluorophenyl)-1-((5-(trifluoromethyl)-1H-pyrrol-3-yl)sulfonyl)pyrrolidin-3-yl)carbamate;
4-((3-benzhydrylazetidin-1-yl)sulfonyl)-2-(trifluoromethyl)-1H-pyrrole;
1-phenyl-4-((5-(trifluoromethyl)-1H-pyrrol-3-yl)sulfonyl)-1,4-diazepane;
1-(bis(4-fluorophenyl)methyl)-4-((5-(trifluoromethyl)-1H-pyrrol-3-yl)sulfonyl)piperazine;
1-((4-chlorophenyl)(phenyl)methyl)-4-((5-(trifluoromethyl)-1H-pyrrol-3-yl)sulfonyl)piperazine;
1-(bis(4-chlorophenyl)methyl)-4-((5-(trifluoromethyl)-1H-pyrrol-3-yl)sulfonyl)piperazine;
1-((2-chlorophenyl)(phenyl)methyl)-4-((5-(trifluoromethyl)-1H-pyrrol-3-yl)sulfonyl)piperazine;
1-(phenyl(pyridin-2-yl)methyl)-4-((5-(trifluoromethyl)-1H-pyrrol-3-yl)sulfonyl)piperazine;
1-(5-bromo-2-methylphenyl)-4-((5-(trifluoromethyl)-1H-pyrrol-3-yl)sulfonyl)piperazine;
6-bromo-2-((5-(trifluoromethyl)-1H-pyrrol-3-yl)sulfonyl)-1,2,3,4-tetrahydroisoquinoline;
7-bromo-2-((5-(trifluoromethyl)-1H-pyrrol-3-yl)sulfonyl)-1,2,3,4-tetrahydroisoquinoline;
3-chloro-N-(4-fluorophenyl)-4-(4-((5-(trifluoromethyl)-1H-pyrrol-3-yl)sulfonyl)piperazin-1-yl)benzamide;
1-(2-(1-methyl-1H-pyrazol-4-yl)phenyl)-4-((5-(trifluoromethyl)-1H-pyrrol-3-yl)sulfonyl)piperazine;
1-(2-methyl-5-(pyridin-4-yl)phenyl)-4-((5-(trifluoromethyl)-1H-pyrrol-3-yl)sulfonyl)piperazine;
5-(2-fluoro-4-methylphenyl)-1-((5-(trifluoromethyl)-1H-pyrrol-3-yl)sulfonyl)-1,2,3,4-tetrahydroquinoline;
N,N-dimethyl-4-(1-((5-(trifluoromethyl)-1H-pyrrol-3-yl)sulfonyl)-1,2,3,4-tetrahydroquinolin-5-yl)aniline;
5-(chroman-6-yl)-1-((5-(trifluoromethyl)-1H-pyrrol-3-yl)sulfonyl)-1,2,3,4-tetrahydroquinoline;
5-(o-tolyl)-1-((5-(trifluoromethyl)-1H-pyrrol-3-yl)sulfonyl)-1,2,3,4-tetrahydroquinoline;
methyl 3-(1-((5-(trifluoromethyl)-1H-pyrrol-3-yl)sulfonyl)-1,2,3,4-tetrahydroquinolin-5-yl)benzoate;
6-phenyl-2-((5-(trifluoromethyl)-1H-pyrrol-3-yl)sulfonyl)-1,2,3,4-tetrahydroisoquinoline;
(R)-4-(5-chloro-2-methylphenyl)-2-methyl-1-((5-(trifluoromethyl)-1H-pyrrol-3-yl)sulfonyl)piperazine;
(S)-4-(5-chloro-2-methylphenyl)-2-methyl-1-((5-(trifluoromethyl)-1H-pyrrol-3-yl)sulfonyl)piperazine;
1-(5-chloro-2-methylphenyl)-2-methyl-4-((5-(trifluoromethyl)-1H-pyrrol-3-yl)sulfonyl)piperazine;
4-(5-chloro-2-methylphenyl)-2,6-dimethyl-1-((5-(trifluoromethyl)-1H-pyrrol-3-yl)sulfonyl)piperazine;
(4-(5-chloro-2-methylphenyl)-1-((5-(trifluoromethyl)-1H-pyrrol-3-yl)sulfonyl)piperazin-2-yl)methanol;
(1S,4S)-2-(4-chloro-2-methylphenyl)-5-((5-(trifluoromethyl)-1H-pyrrol-3-yl)sulfonyl)-2,5-diazabicyclo[2.2.1]heptane;
2-(5-chloro-2-methylphenyl)-6-((5-(trifluoromethyl)-1H-pyrrol-3-yl)sulfonyl)-2,6-diazaspiro[3.3]heptane;
3-(5-chloro-2-methylphenyl)-7-((5-(trifluoromethyl)-1H-pyrrol-3-yl)sulfonyl)-3,7-diazabicyclo[4.2.0]octane;
(S)-2-methyl-4-(2-methyl-5-(pyridin-3-yl)phenyl)-1-((5-(trifluoromethyl)-1H-pyrrol-3-yl)sulfonyl)piperazine;
4-(phenyl(4-((5-(trifluoromethyl)-1H-pyrrol-3-yl)sulfonyl)piperazin-1-yl)methyl)benzonitrile;
1-(bis(4-bromophenyl)methyl)-4-((5-(trifluoromethyl)-1H-pyrrol-3-yl)sulfonyl)piperazine;
4-((4-chlorophenyl)(4-((5-(trifluoromethyl)-1H-pyrrol-3-yl)sulfonyl)piperazin-1-yl)methyl)benzonitrile;
1-((4-chlorophenyl)(4-fluorophenyl)methyl)-4-((5-(trifluoromethyl)-1H-pyrrol-3-yl)sulfonyl)piperazine;
1-((4-bromophenyl)(phenyl)methyl)-4-((5-(trifluoromethyl)-1H-pyrrol-3-yl)sulfonyl)piperazine;
4,4′-((4-((5-(trifluoromethyl)-1H-pyrrol-3-yl)sulfonyl)piperazin-1-yl)methylene)dibenzonitrile;
1-((4-fluorophenyl)(phenyl)methyl)-4-((5-(trifluoromethyl)-1H-pyrrol-3-yl)sulfonyl)piperazine;
1-((2-bromophenyl)(phenyl)methyl)-4-((5-(trifluoromethyl)-1H-pyrrol-3-yl)sulfonyl)piperazine;
1-((4-chlorophenyl)(4-(trifluoromethyl)phenyl)methyl)-4-((5-(trifluoromethyl)-1H-pyrrol-3-yl)sulfonyl)piperazine;
1-((2-fluorophenyl)(phenyl)methyl)-4-((5-(trifluoromethyl)-1H-pyrrol-3-yl)sulfonyl)piperazine;
1-((4-fluorophenyl)(4-methoxyphenyl)methyl)-4-((5-(trifluoromethyl)-1H-pyrrol-3-yl)sulfonyl)piperazine;
1-(di-p-tolylmethyl)-4-((5-(trifluoromethyl)-1H-pyrrol-3-yl)sulfonyl)piperazine;
1-([1,1′-biphenyl]-4-yl(phenyl)methyl)-4-((5-(trifluoromethyl)-1H-pyrrol-3-yl)sulfonyl)piperazine;
1-(phenyl(o-tolyl)methyl)-4-((5-(trifluoromethyl)-1H-pyrrol-3-yl)sulfonyl)piperazine;
1-(di-o-tolylmethyl)-4-((5-(trifluoromethyl)-1H-pyrrol-3-yl)sulfonyl)piperazine;
1-(bis(3-methoxyphenyl)methyl)-4-((5-(trifluoromethyl)-1H-pyrrol-3-yl)sulfonyl)piperazine;
1-((3-bromophenyl)(phenyl)methyl)-4-((5-(trifluoromethyl)-1H-pyrrol-3-yl)sulfonyl)piperazine;
1-((4-(methylsulfonyl)phenyl)(phenyl)methyl)-4-((5-(trifluoromethyl)-1H-pyrrol-3-yl)sulfonyl)piperazine;
1-(phenyl(thiophen-2-yl)methyl)-4-((5-(trifluoromethyl)-1H-pyrrol-3-yl)sulfonyl)piperazine;
1-(9H-fluoren-9-yl)-4-((5-(trifluoromethyl)-1H-pyrrol-3-yl)sulfonyl)piperazine;
methyl 4-(phenyl(4-((5-(trifluoromethyl)-1H-pyrrol-3-yl)sulfonyl)piperazin-1-yl)methyl)benzoate;
4-(phenyl(4-((5-(trifluoromethyl)-1H-pyrrol-3-yl)sulfonyl)piperazin-1-yl)methyl)benzoic acid;
1-(cyclopentyl(phenyl)methyl)-4-((5-(trifluoromethyl)-1H-pyrrol-3-yl)sulfonyl)piperazine;
1-(2-ethyl-1-phenylbutyl)-4-((5-(trifluoromethyl)-1H-pyrrol-3-yl)sulfonyl)piperazine;
1-((4-chlorophenyl)(cyclobutyl)methyl)-4-((5-(trifluoromethyl)-1H-pyrrol-3-yl)sulfonyl)piperazine;
1-((4-chlorophenyl)(cyclohexyl)methyl)-4-((5-(trifluoromethyl)-1H-pyrrol-3-yl)sulfonyl)piperazine;
1-(2-methyl-1-phenylpropyl)-4-((5-(trifluoromethyl)-1H-pyrrol-3-yl)sulfonyl)piperazine;
1-(cyclopropyl(4-(trifluoromethyl)phenyl)methyl)-4-((5-(trifluoromethyl)-1H-pyrrol-3-yl)sulfonyl)piperazine;
N,2-diphenyl-2-(4-((5-(trifluoromethyl)-1H-pyrrol-3-yl)sulfonyl)piperazin-1-yl)acetamide;
N-benzyl-2-phenyl-2-(4-((5-(trifluoromethyl)-1H-pyrrol-3-yl)sulfonyl)piperazin-1-yl)acetamide;
4-(phenyl(4-((5-(trifluoromethyl)-1H-pyrrol-3-yl)sulfonyl)piperazin-1-yl)methyl)benzaldehyde;
(4-(phenyl(4-((5-(trifluoromethyl)-1H-pyrrol-3-yl)sulfonyl)piperazin-1-yl)methyl)phenyl)methanol;
4-(phenyl(4-((5-(trifluoromethyl)-1H-pyrrol-3-yl)sulfonyl)piperazin-1-yl)methyl)benzamide;
1-((4-(1H-tetrazol-5-yl)phenyl)(phenyl)methyl)-4-((5-(trifluoromethyl)-1H-pyrrol-3-yl)sulfonyl)piperazine;
N-(4-(phenyl(4-((5-(trifluoromethyl)-1H-pyrrol-3-yl)sulfonyl)piperazin-1-yl)methyl)benzyl)-5-(trifluoromethyl)-1H-pyrrole-3-sulfonamide;
(4-(phenyl(4-((5-(trifluoromethyl)-1H-pyrrol-3-yl)sulfonyl)piperazin-1-yl)methyl)phenyl)methanamine;
1-methyl-3-(4-(phenyl(4-((5-(trifluoromethyl)-1H-pyrrol-3-yl)sulfonyl)piperazin-1-yl)methyl)benzyl)urea;
N-(4-(phenyl(4-((5-(trifluoromethyl)-1H-pyrrol-3-yl)sulfonyl)piperazin-1-yl)methyl)benzyl)isonicotinamide; and
N-methyl-1-(4-(phenyl(4-((5-(trifluoromethyl)-1H-pyrrol-3-yl)sulfonyl)piperazin-1-yl)methyl)phenyl)methanamine;
or a pharmaceutically acceptable salt thereof.

53.-55. (canceled)

56. A pharmaceutical composition comprising the compound of claim 1, or a pharmaceutically acceptable salt thereof, and a pharmaceutically acceptable carrier.

57.-58. (canceled)

59. A method of treating a disease or disorder associated with USP18 in a patient, comprising administering to the patient a therapeutically effective amount of the compound of claim 1, or a pharmaceutically acceptable salt thereof.

60.-62. (canceled)


SEQUENCE LISTING (1 sequence)

SEQ ID NO: 1 - protein, 589 aa, synthetic construct
  source 1..589: note = His6-GST-3C-hUSP18
MHHHHHHSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKLEVLFQGPLAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC